SEQUENCE LISTING (15 sequences)

SEQ ID NO: 1 - DNA, 601 bp, Homo sapiens
ggaggacccagaaatccacgtggcatggatattacacatgctcccagactcacagctctattcacacactggcttagatataaacatgggccatgtgcacataccagacacccagatttccatagcgcatgccccagacatgcacaggtttccatgtatacacagatctattaatgatgttgtaataataaccaayatttattgggtgcttgatatgtgccaggtattattttaaacacttcatatgaattgctatttagtcctcacaacaaccatatgggacatacgaatyattatctcygttttagagatgaagacactaaggcccaaagaagtcacatatcttgcccaaggtcatgtgcacagcaagtaaggggcagaagtgggatttgaactcatgcagtctggctacagagcctaagctccttgttaccacaccatttagtgtatttaccactgatgctcagctgccgtctggtacacaatcccacagaaatgcaccaatatcaatagaatgtactgataatgtgtatatcatgtgtgtactcccaggcatgtacacgtgggtgcctaaatatcatagagtgcatactgtatatca

SEQ ID NO: 2 - DNA, 601 bp, Homo sapiens
ggcccaggagggattggaggtgcagcygccaccaccagcaggggcttcaggctgagtgatgagggaggtgcagaggggaggaggctgcacagttcctaccaccttatgcaagagggagaacaaaggggctttgctgggaggtattagaagattaagtggctgagtccaggacaaactcagcaagccccttgggacctgaattagcaatagcaactgaaagcggggaattaaagctcctggccttcgagcagaggccccccaccccttttagtctctgcctgggcctcctcagagagctaayctgctgccctgcctgagttcttgcctgggggctgtgtgaggggctgttccttcyacccagaggcaactggagccttcctggggaaggagctgcctgacccctgacccctgacctttgggcacaggctccgagcaggctgggtggcccaatttggactcagagctgagtcccgcctctcgactccacacttggctgagctgtgagtttgagagaaaccactgctcctctctgggcctctccttcctgctaataaagtgggaatctggggccatggactcaatggccatcccaaggagtcct

SEQ ID NO: 3 - DNA, 52 bp, Homo sapiens
atatgggacatacgaatcattatctcygttttagagatgaagacactaaggc

SEQ ID NO: 4 - DNA, 52 bp, Homo sapiens
cataggacagattctagattttccttrtgatacagagaaatataagacataa

SEQ ID NO: 5 - DNA, 47 bp, Homo sapiens
  modified_base (27)..(27): May or may not be present
cttttacggcctcctccgtcactacckggcgagtgattcagcctgcc

SEQ ID NO: 6 - DNA, 52 bp, Homo sapiens
ttgttggcatcgggggaaaggaaataygatctaaggtaagtagaattttaaa

SEQ ID NO: 7 - DNA, 52 bp, Homo sapiens
cagtgtcattcatccatgctattccastaggtacgctttcttcaacagccac

SEQ ID NO: 8 - DNA, 52 bp, Homo sapiens
aattcaggacaaatcagcagattttayaggactacatgtggaaaaaatctct

SEQ ID NO: 9 - DNA, 52 bp, Homo sapiens
ttgtctcaaaaaatttttaaatttccytcttaatttcttctttgactcactg

SEQ ID NO: 10 - DNA, 52 bp, Homo sapiens
tataacatgttaaagtatttataaacrtattccctgaaacagctattgtagc

SEQ ID NO: 11 - DNA, 52 bp, Homo sapiens
taagcttcattgtaaatgcttggtgtmcattctatggaattgacaaattata

SEQ ID NO: 12 - DNA, 52 bp, Homo sapiens
caccaggagaaaaaaaaatgtgtgctkcaggggtcgggggagtgggtaggta

SEQ ID NO: 13 - DNA, 52 bp, Homo sapiens
aaattctaaggaagtgaataaacatgscctttatgtaattactactgaggag

SEQ ID NO: 14 - DNA, 52 bp, Homo sapiens
acaaaaagataataaattactatcatygttggatattttctgtcaaactcag

SEQ ID NO: 15 - DNA, 52 bp, Homo sapiens
ctgcctgggcctcctcagagagctaayctgctgccctgcctgagttcttgcc

(19) United States
(12) Patent Application Publication
(10) Pub. No.: US 2014/0087960 A1
(43) Pub. Date: Mar. 27, 2014

(54) Markers Related to Age-Related Macular Degeneration and Uses Therefor

(71) Applicants: Johanna M. Seddon, Boston, MA (US); Mark Daly, Arlington, MA (US)

(72) Inventors: Johanna M. Seddon, Boston, MA (US); Mark Daly, Arlington, MA (US)

(21) Appl. No.: 13/992,378

(22) PCT Filed: Dec. 14, 2011
(86) PCT No.: PCT/US11/64936

§ 371 Date: Dec. 9, 2013

Related U.S. Application Data
(60) Provisional application No. 61/529,817, filed on Aug. 31, 2011.
(60) Provisional application No. 61/444,482, filed on Feb. 18, 2011.
(60) Provisional application No. 61/422,905, filed on Dec. 14, 2010.

(51) Int. Cl. C12Q 1/68

(52) U.S. Cl. CPC C12Q 1/6883
USPC 506/9; 435/6.11; 506/16

(57) ABSTRACT

Methods are provided of screening for age-related macular degeneration (AMD), including a risk of a subject devel - oping AMD or a risk of a subject progressing to an advanced fom of AMD. The methods can include analyzing a sample obtained from the subject for the presence of at least one single nucleotide polymorphism (SNP) selected from the group consisting of rs4711751, rs6982567, rs1999930, rs13278062, rs1912795, rs2270637, rs12040406, rs1367068, rs1079982, rs1443179, rs7720497, and/or rs6 1800454.

### REFERENCE TO RELATED APPLICATIONS

This application claims priority to and the benefit of U.S. Provisional Application No. 61/422,905, filed on Dec. 14, 2010; U.S. Provisional Application No. 61/444,482, filed on Feb. 18, 2011; and U.S. Provisional Application No. 61/529,817, filed on Aug. 31, 2011. The entire disclosure of each of the above-identified applications is incorporated by reference.

### STATEMENT REGARDING FEDERALLY FUNDED RESEARCH

This invention was made with government support under grant number RO1 EY11309 awarded by the National Institutes of Health and the National Eye Institute. The government has certain rights in the invention.

### BACKGROUND

Age-related macular degeneration (AMD) is the most common geriatric eye disorder leading to blindness. Macular degeneration is responsible for visual handicap in what is estimated conservatively to be approximately 16 million individuals worldwide. Among the elderly, the overall prevalence is estimated between 5.7% and 30% depending on the definition of early AMD, and its differentiation from features of normal aging, a distinction that remains poorly understood.

Histopathologically, the hallmark of early neovascular AMD is accumulation of extracellular drusen and basal laminar deposit (abnormal material located between the plasma membrane and basal lamina of the retinal pigment epithelium) and basal linear deposit (material located between the basal lamina of the retinal pigment epithelium and the inner collageneous zone of Bruch's membrane). The end stage of AMD is characterized by a complete degeneration of the neurosensory retina and of the underlying retinal pigment epithelium in the macular area. Advanced stages of AMD can be subdivided into geographic atrophy and exudative AMD. Geographic atrophy is characterized by progressive atrophy of the retinal pigment epithelium. In exudative AMD the key phenomenon is the occurrence of choroidal neovascularisation (CNV). Eyes with CNV have varying degrees of reduced visual acuity, depending on location, size, type and age of the neovascular lesion. The development of choroidal neovascular membranes can be considered a late complication in the natural course of the disease possibly due to tissue disruption (Bruch's membrane) and decompensation of the underlying longstanding processes of AMD.

Many pathophysiological aspects as well as vascular and environmental risk factors are associated with a progression of the disease. Family, twin, segregation, and case-control studies all suggested an involvement of genetic factors in the etiology of AMD prior to the discovery of various genes associated with AMD.

Knowledge is growing about the extent of heritability, number of genes involved, and mechanisms underlying phenotypic heterogeneity. The search for genes and markers related to AMD faces challenges—onset is late in life, and there is usually only one generation available for studies. The parents of patients are often deceased, and their children are too young to manifest the disease. Generally, the heredity of late-onset diseases has been difficult to estimate because of the uncertainties of the diagnosis in previous generations and the inability to diagnose AMD among the children of an affected individual. Even in the absence of the ambiguities in the diagnosis of AMD in previous generations, the late onset of the condition itself, natural death rates, and small family sizes result in underestimation of genetic forms of AMD, and in overestimation of rates of sporadic disease. Moreover, the phenotypic variability is considerable, and it is conceivable that the currently used diagnostic entity of AMD in fact represents a spectrum of underlying conditions with various genetic and environmental factors involved.

There remains a strong need for improved methods of diagnosing or prognosticating AMD or a susceptibility to AMD in subjects, as well as for evaluating and developing new methods of treatment.

### SUMMARY

The application relates, in part, to the identification of numerous genetic markers which are associated with the presence or progression of age-related macular degeneration (AMD) in an individual. More specifically, methods are provided for diagnosing a risk of an individual developing AMD or progressing to advanced forms of AMD (e.g., geographic atrophy and/or wet AMD) using these genetic markers.

For example, in one aspect the invention provides a method of screening for age-related macular degeneration (AMD) in a human subject. The method can include determining a risk of AMD progression in the subject by analyzing a sample obtained from the subject for the presence in the subject's genome of at least one single nucleotide polymorphism (SNP) identified in Tables 3-10, or a proxy therefor. In some embodiments, a proxy is a marker that is in linkage disequilibrium with a particular SNP or marker of interest. The presence of a SNP indicates that the subject has an increased risk of developing AMD or developing an advanced form of AMD. The markers can be used individually or in combination when screening a subject. Preferred SNPs include, but are not limited to, rs4711751 (VEGF), rs1999930 (COL10A1/FRK), rs13278062 (TNFRSF10A), rs 1912795 (B3GALTL), rs2270637 (SLC18A1), rs6982567 (GDF6), rs12040406 and rs1367068 (CD55), rs1079982 (CARD10), rs1443179 (INTU), rs7720497 (ADAMTS16), and rs61800454 (TMCO1). In some embodiments, the presence of a particular SNP indicates the subject has an increased risk of developing AMD. In some embodiments, the presence of a particular SNP indicates the subject has an increased risk of developing an advanced form of AMD, such as geographic atrophy and/or wet AMD, which also is referred to as neovascular disease, choroidal neovascularisation (CNV), and exudative AMD.

Various techniques can be used for analyzing a sample to determine the presence of a SNP in the subject's genome. For example, in some embodiments, the method of screening can include the steps of (i) combining a nucleic acid sample from the subject with one or more polynucleotide probes capable of hybridizing selectively to a particular SNP (e.g., any SNP identified in Tables 3-10) or gene allele, or a proxy therefor, and (ii) detecting the presence or absence of hybridization. The probes can be oligonucleotides capable of priming polynucleotide synthesis in an amplification reaction, such as PCR or real time PCR. In some embodiments, the presence of at least one SNP is determined using a microarray. In various embodiments, the presence of at least one SNP is determined by sequencing a portion of the patient's genome.

In some embodiments, the patient is asymptomatic at the time of screening for AMD, and in some embodiments, the patient displays one or more AMD like symptoms at the time of screening.

In some embodiments, the method includes detecting a haplotypes that includes a particular SNP (e.g., any SNP listed in Tables 3-10).

In some embodiments, the method includes screening for a specific subtype of AMD, such as, for example, early AMD, geographic atrophy, wet AMD, neovascular disease, choroidal neovascularisation (CNV), exudative AMD, and combinations thereof.

The invention also provides, in part, a diagnostic system. The diagnostic system can include an array of polynucleotides comprising one or more of SEQ ID NOS:1-15, or any reference sequences corresponding to the SNPs identified in Tables 2-10. The polynucleotides can include at least six or more contiguous nucleotides, and the polynucleotides can include an allelic polymorphism or SNP. The system also can include an array reader, an image processor, a database having AMD allelic data records and patient information records, a processor, and an information output. The system compiles and processes patient data and outputs information relating to the statistical probability of the patient developing AMD.

The system can be used for various methods, including contacting a subject sample or portion thereof to the diagnostic array under high stringency hybridization conditions; inputting patient information into the system; and obtaining from the system information relating to the statistical probability of the patient developing AMD.

Further provided are methods for diagnosing risk of AMD or severe forms of AMD in a human subject. The method includes combining genetic risk with behavioral risk, wherein the genetic risk is determined by detecting in a sample obtained from a subject the presence or absence of a single nucleotide polymorphism SNP listed in Tables 3, 4, 5, 6, 7, 8, 9, or 10, or proxy therefor, wherein the presence of the allele is indicative of an increased risk of the subject developing AMD or a severe form of AMD. In various embodiments, behavioral risk is assessed by determining if the subject exhibits a behavior or trait selected from: obesity, smoking, vitamin and dietary supplement intake, use of alcohol or drugs, poor diet, a sedentary lifestyle, medical history of heart disease or other vascular disease, and medical history of kidney or liver disease.

### BRIEF DESCRIPTION OF DRAWINGS

The figures are not necessarily to scale, emphasis instead generally being placed upon illustrative principles. The figures are to be considered illustrative in all aspects and are not intended to limit the invention, the scope of which is defined only by the claims.

FIGS. 1 and 2 are nucleic acid sequences of VEGFA and GDF6 SNPs, respectively, in accordance with an illustrative embodiment.

FIGS. 3A and 3B are graphs showing a preliminary χ2 association analysis, in accordance with an illustrative embodiment.

FIG. 4 is a graph showing 80% power to detect a biallelic CNV, in accordance with an illustrative embodiment.

FIGS. 5.1 and 5.2 are nucleic acid sequences of various SNPs, in accordance with an illustrative embodiment.

FIGS. 6a-d show the FRI/COL10A1 region and VEGFA region, and association with AMD, in accordance with an illustrative embodiment.

FIG. 7 shows distribution of genetic ancestry estimated by EIGENSOFT, in accordance with an illustrative embodiment.

FIG. 8 shows quantile-quantile (Q;Q) plots, in accordance with an illustrative embodiment.

FIG. 9 shows a Manhattan-Plot, in accordance with an illustrative embodiment.

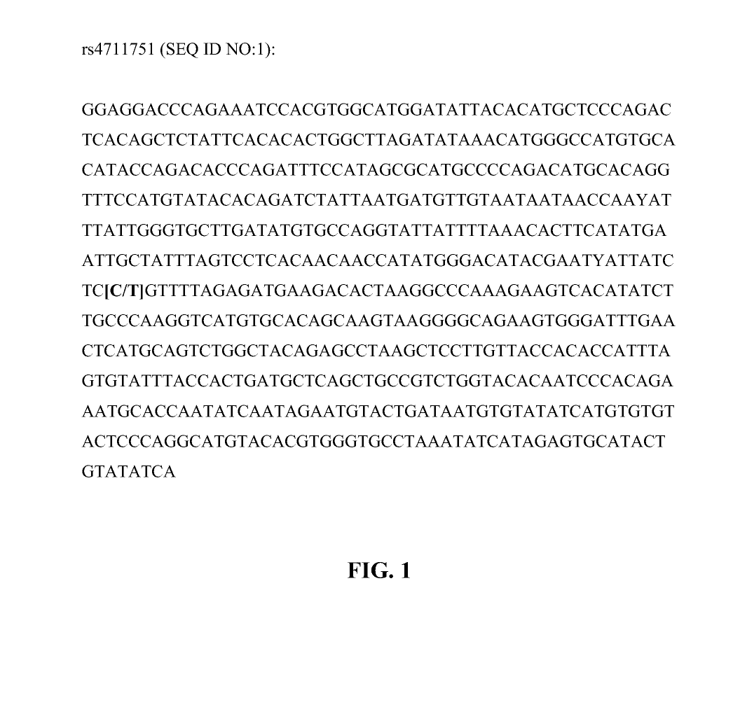

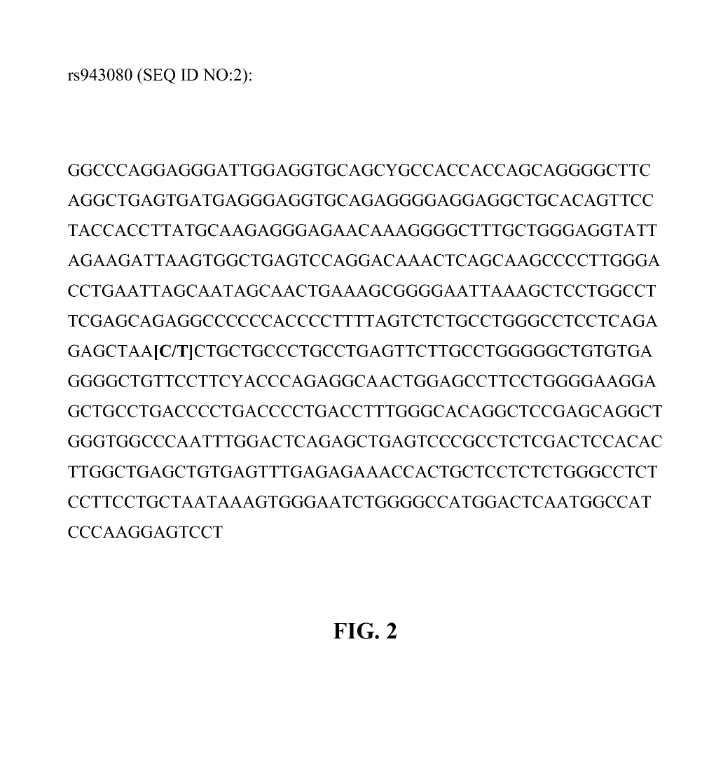

### DETAILED DESCRIPTION

The present invention relates, in part, to the discovery that particular alleles at polymorphic sites associated with genes, including alpha chain of type X collagen (COL10A1), vascular endothelial growth factor A (VEGFA) and growth/differentiation factor 6 (GDF6) are useful as markers for AMD etiology, for determining susceptibility to AMD, and for predicting or monitoring disease progression or severity, e.g., to determine a treatment course and/or to titrate dosages of therapeutic agents. More specifically, and by non-limiting example, the single nucleotide polymorphisms (SNPs) rs4711751 in the VEGFA gene and rs6982567 in the GDF6 gene can be used as markers for AMD etiology, for determining susceptibility to AMD, and for predicting disease progression or severity, and for distinguishing risk of geographic atrophy, the advanced dry type of AMD from the advanced wet form of AMD. In addition, Tables 3, 4, 5, 6, 7, 8, 9, and 10 list additional polymorphisms that are also useful as such markers. Furthermore, genes and/or markers in linkage disequilibrium with these SNPs provide additional such markers.

As used herein, “gene” is a term used to describe a genetic element that gives rise to expression products (e.g., pre-mRNA, mRNA and polypeptides). A gene can include regulatory elements, exons and sequences that otherwise appear to have only structural features, e.g., introns and untranslated regions.

The genetic markers disclosed herein are particular “alleles” at “polymorphic sites” associated with various genes, including VEGFA, GFD6, and any markers identified in tables 3-10. A nucleotide position at which more than one nucleotide can be present in a population (either a natural population or a synthetic population, e.g., a library of synthetic molecules), is referred to herein as a “polymorphic site”. Where a polymorphic site is a single nucleotide in length, the site is referred to as a single nucleotide polymorphism (“SNP”). If at a particular chromosomal location, for example, one member of a population has an adenine and another member of the population has a thymine at the same genomic position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

A genetic marker is “associated” with a genetic element or phenotypic trait, for example, if the marker is co-present with the genetic element or phenotypic trait at a frequency that is higher than would be predicted by random assortment of alleles (based on the allele frequencies of the particular population). Association also indicates physical association, e.g., proximity in the genome or presence in a haplotype block, of a marker and a genetic element.

A reference sequence is typically referred to for a particular genetic element, e.g., a gene. The reference sequence, often chosen as the most frequently occurring allele, is referred to as a “wild type” allele or the “major allele”). Alleles that are more common or less common in individuals with a disease/trait compared to individuals without the disease/trait, with a certain level of statistical significance, are referred to as the variant alleles. The corresponding genotype is referred to as a genetic variant.

Some variant alleles can include changes that affect a polypeptide or protein, e.g., the polypeptide encoded by a variant allele. These sequence differences, when compared to a reference nucleotide sequence, can include, for example, the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence.

Alternatively, a polymorphism associated with AMD or a susceptibility to AMD can be a synonymous change in one or more nucleotides (i.e., a change that does not result in a change to a codon of a complement pathway gene). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype is a combination or set of genetic markers, e.g., particular alleles at polymorphic sites, such as, e.g., SNPs and/or microsatellites. The haplotypes described herein are associated with AMD and/or a susceptibility to AMD. Detection of the presence or absence of the haplotypes herein, therefore, is indicative of AMD, is indicative of a susceptibility to AMD, is indicative of a factor related to progression from early to intermediate or late stages of AMD, is indicative of progression from intermediate to late stages of AMD, or is indicative of a lack of AMD. Detecting haplotypes, therefore, can be accomplished by methods known in the art for detecting sequences at polymorphic sites.

“Linkage” refers to a higher than expected statistical association of genotypes and/or phenotypes with each other. Linkage disequilibrium (“LD”) refers to a non-random assortment of two genetic elements. If a particular genetic element (e.g., an allele at a polymorphic site), for example, occurs in a population at a frequency of 0.25 and another occurs at a frequency of 0.25, then the predicted occurrence of a person's having both elements is 0.125, assuming a random distribution of the elements. If, however, it is discovered that the two elements occur together at a frequency higher than 0.125, then the elements are said to be in LD since they tend to be inherited together at a higher frequency than what their independent allele frequencies would predict. Roughly speaking, LD is generally correlated with the frequency of recombination events between the two elements. Allele frequencies can be determined in a population, for example, by genotyping individuals in a population and determining the occurrence of each allele in the population. For populations of diploid individuals, e.g., human populations, individuals will typically have two alleles for each genetic element (e.g., a marker or gene).

The invention is also directed to markers identified in a “haplotype block” or “LD block”. These blocks are defined either by their physical proximity to a genetic element, e.g., a VEGFA, GDF6, or the other markers provided herein, or by their “genetic distance” from the element. Markers and haplotypes identified in these blocks, because of their association with AMD and VEGFA, GDF6, or the markers identified herein, are encompassed by the invention. One of sk111 in the art will appreciate regions of chromosomes that recombine infrequently and regions of chromosomes that are “hotspots”, e.g., exhibiting frequent recombination events, are descriptive of LD blocks. Regions of infrequent recombination events bounded by hotspots will form a block that will be maintained during cell division. Thus, identification of a marker associated with a phenotype, wherein the marker is contained within an LD block, identifies the block as associated with the phenotype. Any marker identified within the block can therefore be used to indicate the phenotype.

Additional markers that are in LD with the markers of the invention or haplotypes are referred to herein as “surrogate” markers (i.e., “proxy” markers). Such a surrogate is a marker for another marker or another surrogate marker. Surrogate markers are themselves markers and are indicative of the presence of another marker, which is in turn indicative of either another marker or an associated phenotype.

Susceptibility for developing AMD includes an asymptomatic patient showing increased risk to develop AMD, and a patient having early or intermediate stages of AMD indicating a progression toward more advanced forms of AMD and expected visual loss. Susceptibility for not developing AMD includes an asymptomatic patient having at least one wild type allele, or a non-risk genotype, or a protective genotype, or a non-risk allele, or a protective allele, or a non-risk haplotype, or a protective haplotype indicates a lack of a predisposition for developing AMD.

Genetic markers (e.g., SNPs) can be detected in nucleic acids (e.g., DNA or mRNA) in any suitable sample source obtained or taken from an individual, including blood, saliva, feces, bone, epithelial cells, endothelial cells, blood cells, and other bodily fluids, cells, and/or tissues.

Table 10 lists representative markers which are associated with AMD. These markers, or markers in linkage disequilibrium with these markers (e.g., R squared=0.2 or higher), can be used as markers for AMD etiology, for determining susceptibility to AMD, and for predicting disease progression or severity, and for distinguishing risk of geographic atrophy, the advanced dry type of AMD from the advanced wet form of AMD.

TABLE 10
Markers of AMD

| SNP | GENE | CHR | BP(hg19) | BP(hg18) | BEST-P |
|---|---|---|---|---|---|
| rs4711751 | VEGF | 6 | 43828582 | 43936560 | 8.7E−09 |
| rs1999930 | COL10A1/FRK | 6 | 116387134 | 116493827 | 1.1E−08 |
| rs13278062 | TNFRSF10A | 8 | 23082971 | 23138916 | 3.8E−06 |
| rs1912795 | B3GALTL | 13 | 31838688 | 30736688 | 3.2E−05 |
| rs2270637 | SLC18A1 | 8 | 20036827 | 20081107 | 3.8E−06 |
| rs6982567 | GDF6 | 8 | 96750281 | 96819457 | 6.1E−06 |
| rs12040406 | CD55 | 1 | 207449304 | 205515927 | 2.8E−07 |
| rs1367068 | | 1 | 207394941 | 205461564 | 2.1E−07 |
| rs1079982 | CARD10 | 22 | 37929759 | 36259705 | 3.0E−06 |
| rs1443179 | INTU | 4 | 128276322 | 128495772 | 1.4E−06 |
| rs7720497 | ADAMTS16 | 5 | 5238813 | 5291813 | 3.5E−06 |
| rs61800454 | TMCO1 | 1 | 165720343 | 163986967 | 5.4E−05 |

### Diagnostic Gene Array

In one aspect, the invention comprises an array of gene fragments, particularly nucleic acids including one or more SNPs given as SEQ ID NOS:1-15 and/or sequences including the SNPs identified in Tables 3, 4, 5, 6, 7, 8, 9, or 10 and probes for detecting the allele at the SNPs of one or more of SEQ ID NOS:1-15 and/or sequences including the SNPs identified in Tables 3, 4, 5, 6, 7, 8, 9, and 10. Polynucleotide arrays provide a high throughput technique that can assay a large number of polynucleotide sequences in a single sample. This technology can be used, for example, as a diagnostic tool to assess the risk potential of developing AMD using the SNPs and probes of the invention. Polynucleotide arrays (for example, DNA or RNA arrays), include regions of usually different sequence polynucleotides arranged in a predetermined configuration on a substrate, at defined x and y coordinates. These regions (sometimes referenced as “features”) are positioned at respective locations (“addresses”) on the substrate. The arrays, when exposed to a sample, will exhibit an observed binding pattern. This binding pattern can be detected upon interrogating the array. For example, all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Arrays can be fabricated by depositing previously obtained biopolymers onto a substrate, or by in situ synthesis methods. The substrate can be any supporting material to which polynucleotide probes can be attached, including but not limited to glass, nitrocellulose, silicon, and nylon. Polynucleotides can be bound to the substrate by either covalent bonds or by non-specific interactions, such as hydrophobic interactions. The in situ fabrication methods include those described in U.S. Pat. No. 5,449,754 for synthesizing peptide arrays, and in U.S. Pat. No. 6,180,351 and WO 98/41531 and the references cited therein for synthesizing polynucleotide arrays. Further details of fabricating biopolymer arrays are described in U.S. Pat. No. 6,242,266; U.S. Pat. No. 6,232,072; U.S. Pat. No. 6,180,351; U.S. Pat. No. 6,171,797; EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Other techniques for fabricating biopolymer arrays include known light directed synthesis techniques. Commercially available polynucleotide arrays, such as Affymetrix GeneChip™, can also be used. Use of the GeneChip™, to detect gene expression is described, for example, in Lockhart et al., Nat. Biotechnol., 14:1675, 1996; Chee et al., Science, 274:610, 1996; Hacia et al., Nat. Gen., 14:441, 1996; and Kozal et al., Nat. Med., 2:753, 1996. Other types of arrays are known in the art, and are sufficient for developing an AMD diagnostic array of the present invention.

To create the arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from the nucleotide sequences shown in SEQ ID NO:1-15 and/or sequences including the SNPs identified in Tables 3, 4, 5, 6, 7, 8, 9, and 10, or the complement thereof. Preferred arrays comprise at least one single-stranded polynucleotide probe comprising at least 6, 7, 8, 9, 10, 11, 12, 13, 14, 15, 16, 17, 18, 19, 20, 25, or 30 or more contiguous nucleotides selected from the nucleotide sequences shown in SEQ ID NO:1-15, and/or sequences including the SNPs identified in Tables 3, 4, 5, 6, 7, 8, 9, and 10, or the complement thereof.

Tissue samples from a subject can be treated to form single-stranded polynucleotides, for example by heating or by chemical denaturation, as is known in the art. The single-stranded polynucleotides in the tissue sample can then be labeled and hybridized to the polynucleotide probes on the array. Detectable labels that can be used include but are not limited to radiolabels, biotinylated labels, fluorophors, and chemiluminescent labels. Double stranded polynucleotides, comprising the labeled sample polynucleotides bound to polynucleotide probes, can be detected once the unbound portion of the sample is washed away. Detection can be visual or with computer assistance. Preferably, after the array has been exposed to a sample, the array is read with a reading apparatus (such as an array “scanner”) that detects the signals (such as a fluorescence pattern) from the array features. Such a reader preferably would have a very fine resolution (for example, in the range of five to twenty microns) for an array having closely spaced features.

The signal image resulting from reading the array can then be digitally processed to evaluate which regions (pixels) of read data belong to a given feature as well as to calculate the total signal strength associated with each of the features. The foregoing steps, separately or collectively, are referred to as “feature extraction” (U.S. Pat. No. 7,206,438). Using any of the feature extraction techniques so described, detection of hybridization of a patient derived polynucleotide sample with one of the AMD markers on the array given as SEQ ID NO:1-15 and/or sequences including the SNPs identified in Tables 3, 4, 5, 6, 7, 8, 9, and 10 identifies that subject as having or not having a genetic risk factor for AMD, as described above.

### System for Analyzing Patient Data

In another aspect, the invention provides a system for compiling and processing patient data, and presenting a risk profile for developing AMD or for the progression to late stages. A computer aided medical data exchange system is preferred. The system is designed to provide high-quality medical care to a patient by facilitating the management of data available to care providers. The care providers will typically include physicians, surgeons, nurses, clinicians, various specialists, and so forth. It should be noted, however, that while general reference is made to a clinician in the present context, the care providers may also include clerical staff, insurance companies, teachers and students, and so forth. The system provides an interface, which allows the clinicians to exchange data with a data processing system. The data processing system is linked to an integrated knowledge base and a database.

The database may be software-based, and includes data access tools for drawing information from the various resources as described below, or coordinating or translating the access of such information. In general, the database will unify raw data into a useable form. Any suitable form may be employed, and multiple forms may be employed, where desired, including hypertext markup language (HTML) extended markup language (XML), Digital Imaging and Communications in Medicine (DICOM), Health Level Seven™ (HL7), and so forth. In the present context, the integrated knowledge base is considered to include any and all types of available medical data that can be processed by the data processing system and made available to the clinicians for providing the desired medical care. In general, data within the resources and knowledge base are digitized and stored to make the data available for extraction and analysis by the database and the data processing system. Even where more conventional data gathering resources are employed, the data is placed in a form that permits it to be identified and manipulated in the various types of analyses performed by the data processing system.

The integrated knowledge base is intended to include one or more repositories of medical-related data in a broad sense, as well as interfaces and translators between the repositories, and processing capabilities for carrying out desired operations on the data, including analysis, diagnosis, reporting, display and other functions. The data itself may relate to patient-specific characteristics as well as to non-patient specific information, as for classes of persons, machines, systems and so forth. Moreover, the repositories may include devoted systems for storing the data, or memory devices that are part of disparate systems, such as imaging systems. As noted above, the repositories and processing resources making up the integrated knowledge base may be expandable and may be physically resident at any number of locations, typically linked by dedicated or open network links. Furthermore, the data contained in the integrated knowledge base may include both clinical data (e.g., data relating specifically to a patient condition) and non-clinical data. Examples of preferred clinical data include patient medical histories, patient serum, plasma, and/or other biomarkers such as blood levels of certain other nutrients, fats, female and male hormones, etc., and cellular antioxidant levels, and the identification of past or current environmental, lifestyle and other factors that predispose a patient to develop AMD. These include but are not limited to various risk factors such as obesity, smoking, vitamin and dietary supplement intake, use of alcohol or drugs, poor diet, a sedentary lifestyle, medical history of heart disease or other vascular disease, and/or medical history of kidney or liver disease. Non-clinical data may include more general information about the patient, such as residential address, data relating to an insurance carrier, and names and addresses or phone numbers of significant or recent practitioners who have seen or cared for the patient, including primary care physicians, specialists, and so forth.

The flow of information can include a wide range of types and vehicles for information exchange. In general, the patient can interface with clinicians through conventional clinical visits, as well as remotely by telephone, electronic mail, forms, and so forth. The patient can also interact with elements of the resources via a range of patient data acquisition interfaces that can include conventional patient history forms, interfaces for imaging systems, systems for collecting and analyzing tissue samples, body fluids, and so forth. Interaction between the clinicians and the interface can take any suitable form, depending upon the nature of the interface. Thus, the clinicians can interact with the data processing system through conventional input devices such as keyboards, computer mice, touch screens, portable or remote input and reporting devices. The links between the interface, data processing system, the knowledge base, the database and the resources typically include computer data exchange interconnections, network connections, local area networks, wide area networks, dedicated networks, virtual private network, and so forth.

In general, the resources can be patient-specific or patient-related, that is, collected from direct access either physically or remotely (e.g., via computer link) from a patient. The resource data can also be population-specific so as to permit analysis of specific patient risks and conditions based upon comparisons to known population characteristics. It should be noted that the resources can generally be thought of as processes for generating data. While many of the systems and resources will themselves contain data, these resources are controllable and can be prescribed to the extent that they can be used to generate data as needed for appropriate treatment of the patient. Exemplary controllable and prescribable resources include, for example, a variety of data collection systems designed to detect physiological parameters of patients based upon sensed signals. Such electrical resources can include, for example, electroencephalography resources (EEG), electrocardiography resources (ECG), electromyography resources (EMG), electrical impedance tomography resources (EIT), nerve conduction test resources, electronystagmography resources (ENG), and combinations of such resources. Various imaging resources also can be controlled and prescribed as necessary. Exemplary eye tests include, for example, electrophysiologic tests, elcetroretinograms, electrooculagrams, retinal angiography, retinal photography, ultrasonography, optical coherence tomography, and other imaging modalities such as autofluorescence. A number of modalities of such resources are currently available, such as, for example, X-ray imaging systems, magnetic resonance (MR) imaging systems, computed tomography (CT) imaging systems, positron emission tomography (PET) systems, fluorography systems, sonography systems, infrared imaging systems, nuclear imaging systems, thermoacoustic systems, and so forth. Imaging systems can draw information from other imaging systems, electrical resources can interface with imaging systems for direct exchange of information (such as for timing or coordination of image data generation, and so forth).

In addition to such electrical and highly automated systems, various resources of a clinical and laboratory nature can be accessible. Such resources may include blood, urine, saliva and other fluid analysis resources, including gastrointestinal, reproductive, urological, nephrological (kidney function), and cerebrospinal fluid analysis system. Such resources can further include polymerase (PCR) chain reaction analysis systems, genetic marker analysis systems, radioimmunoassay systems, chromatography and similar chemical analysis systems, receptor assay systems and combinations of such systems. Histologic resources, somewhat similarly, can be included, such as tissue analysis systems, cytology and tissue typing systems and so forth. Other histologic resources can include immunocytochemistry and histopathological analysis systems. Similarly, electron and other microscopy systems, in situ hybridization systems, and so forth can constitute the exemplary histologic resources. Pharmacokinetic resources can include such systems as therapeutic drug monitoring systems, receptor characterization and measurement systems, and so forth. Again, while such data exchange can be thought of passing through the data processing system, direct exchange between the various resources can also be implemented.

Use of the present system involves a clinician obtaining a patient sample, and evaluation of the presence of a genetic marker in that patient indicating a predisposition (or not) for AMD or its progression, such as one or more of SEQ ID NO:1-15, and/or sequences including the SNPs identified in Tables 3, 4, 5, 6, 7, 8, 9, and 10 alone or in combination with other known risk factors. The clinician or their assistant also obtains appropriate clinical and non-clinical patient information, and inputs it into the system. The system then compiles and processes the data, and provides output information that includes a risk profile for the patient, of developing AMD and/or progressing to advanced forms of AMD.

The present invention thus provides for certain polynucleotide sequences that have been correlated to AMD. These polynucleotides are useful as diagnostics, and are preferably used to fabricate an array, useful for screening patient samples. The array, in a currently most preferred embodiment, is used as part of a laboratory information management system, to store and process additional patient information in addition to the patient's genomic profile. As described herein, the system provides an assessment of the patient's risk for developing AMD, risk for disease progression, and likelihood of disease prevention based on patient controllable factors.

### Kits

The invention relates in part to kits and systems useful for performing the diagnostic methods described herein. The methods described herein can be performed by, for example, diagnostic laboratories, service providers, experimental laboratories, and individuals. The kits can be useful in these settings, among others.

Kits include reagents and materials for obtaining genetic material and assaying one or more markers in a sample from an individual, analyzing the results, diagnosing whether the individual is susceptible to or at risk for developing AMD, monitoring disease progression, and/or determining an appropriate treatment course. For example, in some embodiments, the kit can include a needle, syringe, vial, cotton swap or other apparatus for obtaining and/or containing a sample from an individual. In some embodiments, the kit can include at least one reagent which is used specifically to detect a marker disclosed herein. That is, suitable reagents and techniques readily can be selected by one of skill in the art for inclusion in a kit for detecting or quantifying a marker of interest.

For example, where the marker is a nucleic acid (e.g., DNA or RNA), the kit includes reagents appropriate for detecting nucleic acids using, for example, PCR, hybridization techniques, and microarrays.

Where appropriate, the kit includes: extraction buffers or reagents, amplification buffers or reagents, reaction buffers or reagents, hybridization buffers or reagents, immunodetection buffers or reagents, labeling buffers or reagents, and detection means. The kit can include all or part of the nucleic acids of SEQ ID NOS:1-15 and/or a nucleic acid including a SNP identified in Tables 3, 4, 5, 6, 7, 8, 9, and 10, or a nucleic acid molecule complementary thereto.

Kits can also include a control, which can be a control sample, a reference sample, an internal standard, or previously generated empirical data. The control may correspond to a known allele, e.g., a wild type and/or a variant allele. In addition, a control may be provided for each marker or the control may be a reference (e.g., a wild type and/or variant sequence).

Kits can include one or more containers for each individual reagent. Kits can further include instructions for performing the methods described herein and/or interpreting the results, in accordance with any regulatory requirements. In addition, software can be included in the kit for analyzing the results. Preferably, the kits are packaged in a container suitable for commercial distribution, sale, and/or use.

The following examples are provided for illustration, not limitation.

### Example 1

### Discovery of Genetic Variants Associated with AMD

Age-related macular degeneration (AMD), the leading cause of late onset blindness, arises from retinal damage associated with accumulation of drusen and subsequent atrophy or neovascularization that leads to loss of central vision. The results of a genome-wide association study (GWAS) of 979 advanced AMD cases and 1709 controls using the Affymetrix 6.0 platform with replication in seven additional cohorts (totaling 4337 unrelated cases and unrelated 2077 controls) are presented. These data were combined with the data from the Michigan/Penn/Mayo (MPM) GWAS, which was obtained from a public database, to increase sample size. The Michigan/Penn/Mayo (MPM) GWAS implicated different genes. Analyses of the raw genetic data implicated associated variants in the reference single nucleotide polymorphisms listed in Tables 3, 4, and 5, including the VEGFA gene (discovery P=2.66e-05) and the GDF6 gene (discovery P=6.14e-06). In Tables 3 and 5, for example, the effective allele (EA) and odds ratio (OR) are given for each polymorphism. For example, if T is the effective allele and the OR is 1.2, then the T allele is associated with a 20% higher risk compared to the other allele. If C is the effective allele, and the OR is 0.80, then the C allele is associated with a 20% lower risk compared to the other allele.

In Tables 3-10, the column headers include: SNP, single nucleotide polymorphism; GENE, gene of interest within or near putative interval; Chr, chromosome; BP or POS, base-pair position; EA, effective allele; OR, odds ratio; A1, minor allele; A2, major allele; Meta_P, P value for the association between the minor allele and AMD; Z, weighted average and direction of minor allele signal; and P, P value.

Age-related macular degeneration (AMD) is a common, late-onset disorder that is modified by covariates including smoking and BMI, and has a 3-6 fold higher recurrence ratio in siblings than in the general population. The burden of AMD is clinically significant, causes visual loss, and reduces quality of life. Among individuals age 75 or older, approximately one in four have some sign of this disease, while about one in 15 have the advanced form with visual loss.

Described herein is a study involving 979 cases of advanced AMD in the discovery phase with multiple stages of replication. Samples (e.g., blood samples) were genotyped on the Affymetrix 6.0, platform which contains probes for 906,000 SNPs and an additional 946,000 SNP-invariant probes to enhance copy number variation (CNV) analysis and captures 82% of the variation at an r²>0.8 for Europeans in the 3.1 million SNPs of HapMap phase 2. These data were combined with data with raw genetic data from a public database and conducted imputation using the HapMap phase 3 and the raw genetic data from the publicly available 1000 genomes project. Analyses of the resultant dataset uncovered several new AMD susceptibility loci for AMD. Significant, replicated associations include variations in VEGFA and GFD6, thus revealing novel markers associated with AMD pathogenesis. Additional associated markers include the SNPs listed in Tables 3, 4, 5, 6, 7, 8, 9, and 10.

### Methods

Briefly, a genome-wide association (GWAS) was combined with the MPM results as described more detail below. SNPs were imputed based on the HapMap 3 SNP database, and also imputed results based on pilot data from another public database called “the 1000 Genomes project”. Using this dataset as the discovery sample, VEGFA SNP rs4711751 was found to be in linkage disequilibrium with AMD, with a p value of p=2.66e-05. Another marker, GDF6 SNP rs6982567 also was found to be in linkage disequilibrium with AMD, with a p value of p=6.14e-6. These data were sent to other groups for replication using TaqMan® or Sequenom® assays, and the association was confirmed. Additional markers which are candidates for genetic variants associated with AMD are listed in Tables 3, 4, and 5.

### Study Sample Descriptions

The methods employed in this study conformed to the tenets of the Declaration of Helsinki, received approval from Institutional Review Boards, and informed consent was signed by all participants. Some methods have been described in detail previously. (Neale, et al., “Genome-wide association study of advanced age-related macular degeneration identifies a role of the hepatic lipase gene (LIPC).” Proc Natl Acad Sci USA 107, 7395-400 (2010); Fagerness, et al., “Variation near complement factor I is associated with risk of advanced AMD.” Eur J Hum Genet 17, 100-4 (2009); Maller, et al., “Variation in complement factor 3 is associated with risk of age-related macular degeneration.” Nat Genet 39, 1200-1 (2007); Maller, et al., “Common variation in three genes, including a noncoding variant in CFH, strongly influences risk of age-related macular degeneration.” Nat Genet 38, 1055-9 (2006)) Cases had geographic atrophy or neovascular disease based on fundus photography and ocular examination (Clinical Age-Related Maculopathy Grading System (CARMS) stages 4 and 5). (Seddon, et al., “Evaluation of the clinical age-related maculopathy staging system.” Ophthalmology 113, 260-6 (2006))

Controls were unrelated to cases, 60 years of age or older, and were defined as individuals without macular degeneration, categorized as CARMS stage 1, based on fundus photography and ocular examination. Subjects were derived from ongoing AMD study protocols as described previously.

Tufts/MGH Subjects included in the current GWAS were derived from ongoing AMD study protocols as described previously. (Neale, et al., “Genome-wide association study of advanced age-related macular degeneration identifies a role of the hepatic lipase gene (LIPC).” Proc Natl Acad Sci USA 107, 7395-400 (2010); Maller, et al., “Common variation in three genes, including a noncoding variant in CFH, strongly influences risk of age-related macular degeneration.” Nat Genet 38, 1055-9 (2006); Seddon, et al., “Progression of age-related macular degeneration: association with body mass index, waist circumference, and waist-hip ratio.” Arch Ophthalmol 121, 785-92 (2003); Seddon, et al., “A genomewide scan for age-related macular degeneration provides evidence for linkage to several chromosomal regions.” Am J Hum Genet 73, 780-90 (2003); Seddon, et al., “The US twin study of age-related macular degeneration: relative roles of genetic and environmental influences.” Arch Ophthalmol 123, 321-7 (2005); “A randomized, placebo-controlled, clinical trial of high-dose supplementation with vitamins C and E, beta carotene, and zinc for age-related macular degeneration and vision loss: AREDS report no. 8.” Arch Ophthalmol 119, 1417-36 (2001)) MMAP Subjects included in the current GWAS were obtained from dbGaP (http://dbgap.ncbi.nlm.nih.gov/aa/wga.cgi?page=DUC&view_pdf&stacc=phs000182.v2.p1) and described previously. (Chen, W., et al., “Genetic variants near TIMP3 and high-density lipoprotein-associated loci influence susceptibility to age-related macular degeneration.” Proc Natl Acad Sci USA 107, 7401-6 (2010)) Shared controls from GAIN Schizophrenia Study were obtained from dbGap (http://dbgap.ncbi.nlm.nih.gov/aa/wga.cgi?page=DUC&view_pdf&stacc=phs00002 1.v2.p1) and described (Manolio, T. A., et al., “New models of collaboration in genome-wide association studies: the Genetic Association Information Network.” Nat Genet 39, 1045-51 (2007)) The datasets of Tufts/MGH replication, MIGEN controls, Johns Hopkins University (JHU), Columbia University (COL), Washington University (Wash-U), and Hopital Intercommunal de Creteil (FR-CRET) were included in a previous study. (Neale, et al., “Genome-wide association study of advanced age-related macular degeneration identifies a role of the hepatic lipase gene (UPC).” Proc Natl Acad Sci USA 107, 7395-400 (2010) The datasets Centre for Eye Research Australia (AUS), Genentech, Decode (Iceland) and Rotterdam (ROT) applied the same criteria for the diagnosis and IRB approved protocols of their samples.

### Genotyping Using Genome-Wide Panels

The GWAS genotyping of Tufts/MGH samples and MIGEN samples were performed at the Broad and National Center for Research Resources (NCRR) Center for Genotyping and Analysis using the Affymetrix SNP 6.0 GeneChip (909622 SNPs). (Korn, et al., “Integrated genotype calling and association analysis of SNPs, common copy number polymorphisms and rare CNVs.” Nat Genet 40, 1253-60 (2008)) Shared controls froth GAIN study were also genotyped by using the Affymetrix SNP 6.0 GeneChip. MMAP samples were genotyped by Illumina HumanCNV370v1 Bead Array (ILMN 370, 370404 SNPs). (Chen, W., et al., “Genetic variants near TIMP3 and high-density lipoprotein-associated loci influence susceptibility to age-related macular degeneration.” Proc Natl Acad Sci USA 107, 7401-6 (2010))

### Other Replication Genotyping

Samples from Hopital Intercommunal de Creteil (FR-CRET) and Tufts-replication were genotyped at the Broad Institute Center by the Sequenom iPLEX assay (http://www.sequenom.com/Genetic-Analysis/Applications/iPLEX-Genotyping/iPLEX-Overview.aspx). Samples from Wash-U and AUS were genotyped by the Sequenom iPLEX assay at each respective site. Samples from JHU and COL were genotyped by the TaqMan assay using the ABI PRISM 7900 Sequence Detection System (ABI, Foster City, Calif., USA) (https://products.appliedbiosystems.com/ab/en/US/adirect/ab?cmd=catNavigate2&c atID=601283).

### Quality Control

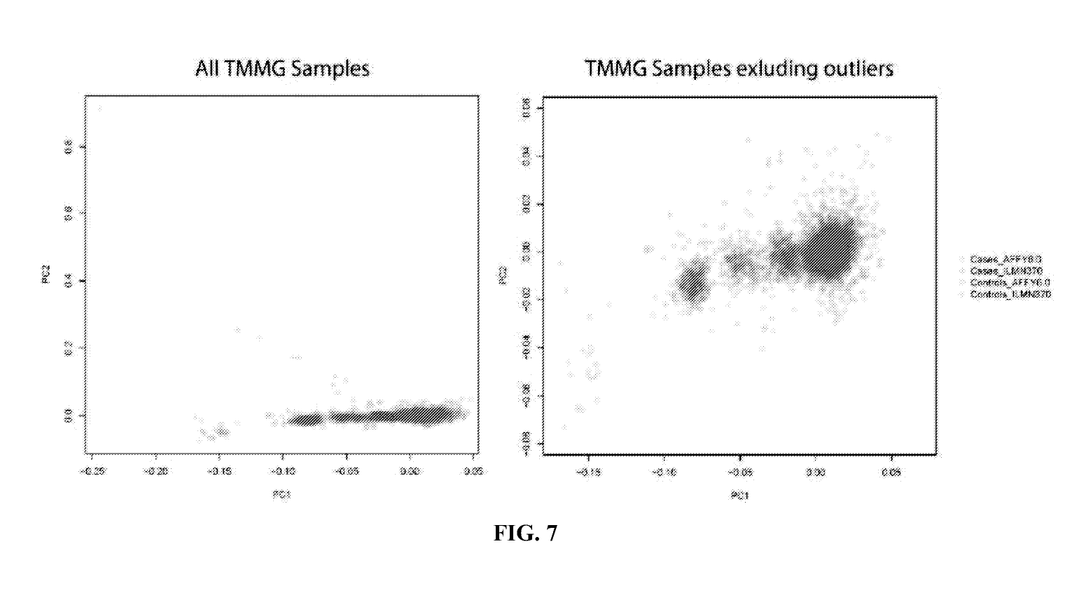

Quality control procedures for the genotype data of Tufts/MGH and MMAP have been described in detail. (Neale, et al., “Genome-wide association study of advanced age-related macular degeneration identifies a role of the hepatic lipase gene (LIPC).” Proc Natl Acad Sci USA 107, 7395-400 (2010); Chen, W., et al., “Genetic variants near TIMP3 and high-density lipoprotein-associated loci influence susceptibility to age-related macular degeneration.” Proc Natl Acad Sci USA 107, 7401-6 (2010)). Briefly, individuals with call rates<0.95 then SNPs with call rates<0.98, Hardy-Weinberg equilibrium P<10⁻⁶, and MAF<0.01 were excluded. Potential relatedness between individuals was identified through a Genome-wide identity-by-state (IBS) matrix using PLINK. (Purcell, et al., “PLINK: a tool set for whole-genome association and population-based linkage analyses.” Am J Hum Genet 81, 559-75 (2007)) IBS was estimated for each pair of individuals and one individual from each duplicate or related pair (pihat>0.2) was removed. Ancestry outliers were identified based on principle components analysis (PCA) using EIGENSOFT (FIG. 7). (Price, et al., “Principal components analysis corrects for stratification in genome-wide association studies.” Nat Genet 38, 904-9 (2006).)

### Imputation and Statistical Analysis

Stringent quality control checks described in Table 13 were applied on each of the data sets contribute to TMMG samples. We next used BEAGLE version 3.0 (Browning, et al., “A unified approach to genotype imputation and haplotype-phase inference for large data sets of trios and unrelated individuals.” Am J Hum Genet 84, 210-23 (2009); Browning, et al., “Rapid and accurate haplotype phasing and missing-data inference for whole-genome association studies by use of localized haplotype clustering.” Am J Hum Genet 81, 1084-97 (2007)) to infer genotypes using the phased CEU and TSI samples (566 haplotypes) of the 1000 Genomes project as a reference. The imputations were performed separately for those cases and controls genotyped on platforms with AFFY 6.0 (more SNPs) and those genotyped with ILMN 370 (less SNPs). For inclusion of data we utilized only imputed genotypes with imputation quality scores>0.6 where the score is defined as the ratio-of-variances (empirical/asymptotic) of each genotype. This score is equivalent to the RSQR_HAT value by MACH and the information content (INFO) measure by PLINK. Since the imputation accuracy are relative low for SNPs with low minor allele frequency (MAF), we only included imputed genotypes of common variants (MAF>0.01) in the analysis. PLINK was used as the primary association test for the imputed genotypes coded by the genotype probabilities for each SNP. The eigenvalue scores with nominal siginificant (p<0.05) association to case/control status (first seven PCAs, PCA 11 and PCA 16) and the original genotyping platform were adjusted as covariates in the association test. The P-value for the combined analysis was derived from the sum of weighted average Z score by the Stouffer's Z-score method as previously described. Neale, et al., “Genome-wide association study of advanced age-related macular degeneration identifies a role of the hepatic lipase gene (LIPC).” Proc Natl Acad Sci USA 107, 7395-400 (2010) The Z score was weighted by the effective sample size of each independent replication cohort if the ratio between cases and controls was equal to 1 based on actual samples listed in Table 12. Heterogeneity of the association between SNP and disease was evaluated by the Cochran's Q-test.

FIG. 7 shows distribution of genetic ancestry along PC1 and PC2 estimated by EIGENSOFT, colored by case (red)/control (blue) status and displayed by the original genotyping platforms, AFFY 6.0 (circle)/ILMN 370 (cross) in all TMMG samples before (left plot) and after (right plot) excluding outliers (PC2>0.05).

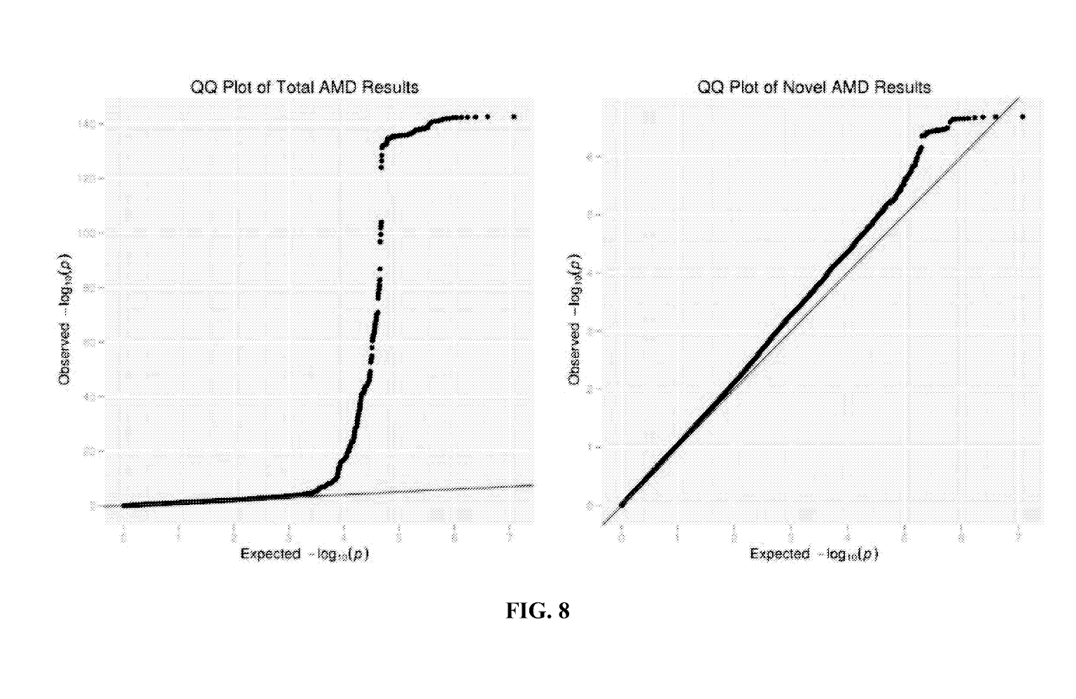

FIG. 8 shows quantile-quantile (Q;Q) plots. We plotted our genome-wide association findings from the cleaned TMMG dataset in Quantile-Quantile (Q:Q) plots. The Q:Q plot on the left represents the strong associations of the CFH, ARMS2/HTRA1, C2/CFB, C3, CFI and LIPC regions that has been previously associated. The Q:Q plot on the right represents the association results of SNPs after excluding these previous associated regions.

FIG. 9 shows a Manhattan-Plot. The log(p-values) of association results from the cleaned TMMG dataset were plotted for SNPs on each chromosome. SNPs with P<5×10⁻⁷ were colored in red and the representative genes for each associated region were labeled.

We genotyped 1242 cases and 492 controls of European ancestry, diagnosed based on fundus photography and ocular examination, 1188 controls from the Myocardial Infarction Genetics Consortium (MIGen), 1378 controls from the GAIN Schizophrenia Study and 1355 cases/1076 controls from the Michigan, Mayo, AREDS, Pennsylvania (MMAP) Cohort Study. After thorough quality control analyses, the merged dataset of Tufts/MMAP/MIGen/GAIN (TMMG) contained 6728 samples, of which 4300 were genotyped by Affymetrix SNP 6.0 GeneChip and 2428 were genotyped by Illumina HumanCNV370v1 Bead Array. The TMMG dataset genotyped by AFFY 6.0 (644,413 SNPs passing quality control checks) was imputed using the phased CEU and TSI samples (566 haplotypes) of the 1000 Genomes project as a reference. (Chen, W., et al., “Genetic variants near TIMP3 and high-density lipoprotein-associated loci influence susceptibility to age-related macular degeneration.” Proc Natl Acad Sci USA 107, 7401-6 (2010)) Separate imputation was performed on the TMMG dataset genotyped on the ILMN 370 (329,315 SNPs passing quality control checks) using the same method. A consensus set of 6,036,699 high quality SNPs from each imputed dataset was analyzed using a generalized linear model controlling for genetic ancestry based on principal component analysis. We observed little statistical inflation in the association statistic after removing known associated loci (see FIGS. 8a, 8b, λ_{gc}=1.047). There were highly statistically significant association signals at SNPs in six previously published loci, including ARMS2/HTRA1 (rs10490924, p=2.5×10⁻¹⁴³), CFH (rs1061170, p=1.6×10⁻¹³⁶) and (rs1410996, p=7.6×10⁻¹³³), CFB (rs641153, p=7.8×10⁻²³), C3 (rs2230199, p=2.6×10⁻¹⁹), C2 (rs9332739, p=7.6×10⁻¹²), CFI (rs10033900, p=8.7×10⁻¹²), and LIPC (rs1532085, p=3.2×10⁻⁷) (FIG. 9).

TABLE 11
Genes associated with AMD in Genome-wide Meta-analysis and analysis of all samples combined.

| | | | | | TMMG | | | | | | | |
|---|---|---|---|---|---|---|---|---|---|---|---|---|
| | | | | | meta-analysis | | | | | | | |
| | | | | | Frequency | | Imputation | | | Combined Analysis | | |
| SNP | GENE | CHR | BP | EA^{#} | Cases | Controls | Quality | OR | P | OR | P | Samples |

Table 11a: Newly identified SNPs associated with AMD susceptibility.

| | | | | | | | | | | | | |
|---|---|---|---|---|---|---|---|---|---|---|---|---|
| rs1999930 | FRK/ | 6 | 116387134 | T | 0.260 | 0.304 | 0.97 | 0.81 | 3.4E−07 | 0.87 | 6.8E−08 | abcdefgh |
| | COL10A1 | | | | | | | | | | | |
| rs4711751 | VEGFA | 6 | 43828582 | T | 0.535 | 0.505 | 0.65 | 1.21 | 1.5E−05 | 1.20 | 2.0E−11 | abdefg |

Table 11b: SNPs previously associated with AMD (genome-wide significant in this study).

| | | | | | | | | | | | | |
|---|---|---|---|---|---|---|---|---|---|---|---|---|
| rs10490924 | HTRA1 | 10 | 124214448 | T | 0.414 | 0.206 | 1.00 | 3.19 | 2.5E−143 | 2.94 | 4.5E−300 | abef |
| rs1061170 | CFH | 1 | 196659237 | C | 0.609 | 0.371 | 1.00 | 2.73 | 1.6E−136 | 2.51 | 3.0E−251 | abefg |
| rs1410996 | CFH | 1 | 196696933 | C | 0.799 | 0.579 | 1.00 | 3.11 | 7.6E−133 | 2.90 | 1.8E−212 | abe |
| rs641153 | CFB | 6 | 31914180 | T | 0.053 | 0.101 | 0.91 | 0.46 | 7.8E−23 | 0.49 | 7.7E−34 | abe |
| rs2230199 | C3 | 19 | 6718387 | G | 0.244 | 0.193 | 0.55 | 1.71 | 2.6E−19 | 1.52 | 1.7E−19 | abe |
| rs9332739 | C2 | 6 | 31903804 | C | 0.023 | 0.045 | 0.89 | 0.45 | 7.6E−12 | 0.50 | 3.0E−16 | abe |
| rs10033900 | CFI | 4 | 110659067 | T | 0.520 | 0.463 | 0.78 | 1.32 | 8.7E−12 | 1.24 | 1.5E−13 | abe |
| rs10468017 | LIPC | 15 | 58678512 | T | 0.257 | 0.287 | 0.90 | 0.84 | 9.7E−05 | 0.85 | 5.3E−09 | abcdef |
| rs3764261 | CETP | 16 | 56993324 | A | 0.360 | 0.328 | 0.97 | 1.16 | 1.2E−04 | 1.16 | 9.6E−09 | abcdef |
| rs9621532* | TIMP3 | 22 | 33084511 | C | 0.037 | 0.051 | 1.00 | 0.72 | 4.9E−04 | 0.64 | 6.0E−14 | abcdef |

*The result of this SNP was from imputation data based on Hapmap 2 project, all other SNPs were imputed based on 1000 Genomes project.
^{#}Effective allele (EA)-frequency and odds ratio based on this SNP for each locus.
(a) TMMG represents Tufts/MMAP/MIGen/GAIN;
(b) Iceland represents deCODE genetics sample replication;
(c) COL represents the Columbia University sample replication;
(d) JHU represents the Johns Hopkins University sample replication;
(e) Genentech represents Genentech of the Roche group sample replication,
(f) WASH-U represents Washington University sample replication;
(g) AUS represents the Centre for Eye Research Australia sample replication;
(h) Rotterdam represents the Rotterdam study sample replication.

TABLE 12
Age-related macular degeneration grade, gender and age information for samples.

| | TMMG | TMMG | | | | | | | |
|---|---|---|---|---|---|---|---|---|---|
| | (AFFY6.0) | (ILMN370) | JHU | COL | AUS | WASH-U | Genentech | Iceland | Rotterdam |
| | N (%) | N (%) | N (%) | N (%) | N (%) | N (%) | N (%) | N (%) | N (%) |
| AMD Grade | | | | | | | | | |
| 1 | 3058 (71) | 1076 (44) | 164 (24) | 368 (33) | 443 (40) | 273 (30) | 8254 (89) | 36949 (98) | 4487 (95) |
| 4 | 465 (11) | 354 (15) | 104 (15) | 210 (19) | 109 (10) | 25 (3) | 44 (1) | 212 (1) | 90 (2) |
| 5 | 777 (18) | 998 (41) | 421 (61) | 523 (48) | 557 (50) | 608 (67) | 918 (10) | 317 (1) | 131 (3) |
| Gender (N %) | | | | | | | | | |
| M | 2116 (49) | 995 (41) | 736 (45) | 417 (40) | 456 (41) | 327 (37) | 4000 (45) | 16190 (43) | 1645 (39) |
| F | 2184 (51) | 1433 (59) | 909 (55) | 631 (60) | 653 (59) | 558 (63) | 4947 (55) | 21288 (57) | 2607 (41) |
| Mean Age by | | | | | | | | | |
| AMD grade | | | | | | | | | |
| 1 | 77 | 74 | 74 | 75 | 71 | 69 | 64 | 54 | 65 |
| 4 | 81 | 79 | 76 | 80 | 71 | N/A | 70 | 84 | 83 |
| 5 | 81 | 80 | 77 | 79 | 77 | 79 | 73 | 84 | 81 |

TMMG (AFFY6.0) represents the genome-wide association data using the Affymetrix 6.0 platform from Tufts Medical Center, Tufts University School of Medicine, with MIGEN and GAIN controls (age not provided);
TMMG (ILMN370) represents the genome-wide association data using the Illumina 370 platform from the Michigan/Penn/Mayo group;
JHU represents the Johns Hopkins University sample replication,
COL represents the Columbia University sample replication,
AUS represents the Centre for Eye Research Australia sample replication,
WASH-U represents Washington University sample replication,
Genentech represents Genentech of the Roche group sample replication,
Iceland represents deCODE genetics sample replication and Rotterdam represents the Rotterdam study sample replication.
AMD Grading System: grade 1 represents individuals with no drusen or a few small drusen, 4 represents individuals with central or non-central geographic atrophy (“advanced dry type”), and 5 represents individuals with neovascular disease (“advanced wet type”).

TABLE 13
The evolution of sample size as a function of the quality control process.

| | TMMG (AFFY 6.0) | | | | TMMG (ILMN 370) | | | |
|---|---|---|---|---|---|---|---|---|
| | | | Change to | | | | Change to | |
| | Cases | Controls | Sample Size | #SNPs | Cases | Controls | Sample Size | #SNPs |
| Initial Sample | 1350 | 506 | — | 909622 | 2159 | 1150 | — | 344942 |
| Addition of Shared MIGEN Controls | 1350 | 1702 | 1196 | 909622 | | | | |
| Initial Dataset Cleaning | 1290 | 1686 | −76 | 690987 | 2159 | 1150 | — | 329368 |
| Addition of Shared GAIN Controls | 1290 | 3064 | 1378 | 666964 | | | | |
| Removal of non-advanced cases | 1290 | 3064 | — | 666964 | 1623 | 1150 | −536 | 329368 |
| Removal of duplicates or relatives | 1244 | 3058 | −52 | 666964 | 1354 | 1081 | −338 | |
| Secondary Dataset Cleaning | 1244 | 3058 | — | 644413 | 1354 | 1081 | — | 329315 |
| Removal of Clustering Outliers | 1242 | 3058 | −2 | 644413 | 1352 | 1076 | −7 | 329315 |

| | Cases | Controls | #SNPs | #SNPs passed QC |
|---|---|---|---|---|
| Combined Sample after imputation | 2594 | 4134 | 7602146 | 6036699 |

Each step represents a cleaning stage. The initial sample represents all samples genotyped or retrieved from dbGaP. The initial dataset cleaning encompasses Hardy-Weinberg equilibrium (HWE), call rate and minor allele frequency threshold. The secondary dataset cleaning reapplied all initial dataset cleaning steps after the addition of shared controls and removed SNPs with differential missingness between cases and controls or SNPs with differential frequencies between different genotyping batches on samples of same disease status. Samples with pihat>0.2 based on a pruned genome-wide SNPs were potential relatives or duplicates. One sample of each relative pair was removed. Individuals who did not cluster with the majority of the sample using a principle components population stratification analysis were removed before imputation. Resultant SNPs from imputation were filtered by quality score >0.6 and Minor allele frequency >0.01.

TABLE 14
Meta-analysis and Combined analysis results for association with AMD - other candidate SNPs tested.

| | | | | | TMMG meta-analysis | | | | | | | |
|---|---|---|---|---|---|---|---|---|---|---|---|---|
| | | | | | Frequency | | Imputation | | | Combined Analysis | | |
| SNP | GENE | CHR | BP | EA^{#} | Cases | Controls | Quality | OR | P | OR | P | Samples |
| rs1883025 | ABCA1 | 9 | 107664301 | T | 0.257 | 0.279 | 0.95 | 0.87 | 1.8E−03 | 0.87 | 9.5E−07 | abcdefh |
| rs13095226 | COL8A1 | 3 | 99396272 | C | 0.123 | 0.104 | 1.00 | 1.22 | 5.9E−04 | 1.18 | 1.3E−05 | abcdefh |
| rs2883171 | CDH12 | 5 | 21273843 | G | 0.137 | 0.116 | 0.69 | 1.32 | 1.6E−05 | 1.22 | 1.9E−05 | abe |
| rs10103808 | FAM135B | 8 | 139143072 | T | 0.115 | 0.090 | 0.96 | 1.31 | 1.7E−05 | 1.22 | 3.3E−05 | abe |
| rs12040406 | C4BPA, CD55 | 1 | 207449304 | C | 0.083 | 0.094 | 0.19 | 0.46 | 4.6E−07 | 0.73 | 9.9E−05 | abcdeg |
| rs4256145 | OTOL1 | 3 | 161710141 | G | 0.306 | 0.338 | 0.99 | 0.84 | 1.9E−05 | 0.90 | 1.3E−04 | abe |
| rs61856267 | PCDH15 | 10 | 56710188 | G | 0.114 | 0.095 | 0.67 | 1.36 | 4.8E−05 | 1.27 | 1.5E−04 | abe |
| rs4967980 | USP31, SCNN1G | 16 | 23183594 | C | 0.256 | 0.225 | 0.93 | 1.20 | 4.4E−05 | 1.14 | 1.7E−04 | abe |
| rs12632671 | TKT | 3 | 53258424 | A | 0.068 | 0.057 | 0.71 | 1.45 | 7.7E−05 | 1.28 | 1.9E−04 | abe |
| rs16965939 | TMCO5 | 15 | 38204793 | T | 0.091 | 0.108 | 0.51 | 0.68 | 3.6E−05 | 0.80 | 2.0E−04 | abcde |
| rs722782 | MYOM2 | 8 | 516479 | A | 0.084 | 0.106 | 0.99 | 0.76 | 1.5E−05 | 0.85 | 3.5E−04 | abe |
| rs1915098 | IGSF11 | 3 | 118198440 | A | 0.141 | 0.166 | 0.95 | 0.80 | 2.7E−05 | 0.88 | 5.8E−04 | abdefg |
| rs72850977 | CTSD | 11 | 1811496 | A | 0.148 | 0.172 | 0.79 | 0.78 | 7.5E−06 | 0.87 | 6.2E−04 | abdef |
| rs2270637 | SLC18A1 | 8 | 20036827 | G | 0.173 | 0.200 | 0.99 | 0.82 | 4.4E−05 | 0.88 | 6.4E−04 | abdeh |
| rs6531212 | SDC1, LAPTM4A | 2 | 20338020 | T | 0.318 | 0.351 | 1.00 | 0.85 | 4.3E−05 | 0.92 | 6.8E−04 | abcdef |
| rs11755724 | RREB1 | 6 | 7118990 | A | 0.343 | 0.372 | 1.00 | 0.88 | 9.3E−04 | 0.93 | 1.1E−03 | abcdefg |
| rs17628762 | CELF4 | 18 | 35871719 | A | 0.382 | 0.343 | 0.98 | 1.19 | 8.3E−06 | 1.08 | 4.6E−03 | abcdef |
| rs10280782 | TRA2A, CLK2P | 7 | 23584282 | G | 0.123 | 0.151 | 0.99 | 0.77 | 2.4E−06 | 0.91 | 6.4E−03 | abcdefg |
| rs2127740 | PSMD7 | 16 | 73732309 | A | 0.139 | 0.114 | 0.96 | 1.29 | 1.0E−05 | 1.14 | 6.4E−03 | abe |
| rs1789110 | MBP | 18 | 74859044 | C | 0.413 | 0.375 | 0.89 | 1.21 | 2.3E−06 | 1.07 | 2.5E−02 | abcdef |
| rs12926103 | FOXF1 | 16 | 86371775 | A | 0.066 | 0.057 | 0.79 | 1.44 | 2.9E−05 | 1.17 | 9.2E−02 | abe |

(a) TMMG;
(b) ICELAND;
(c) COL;
(d) JHU;
(e) Genentech;
(f) Wash-U;
(g) AUS;
(h) ROT. (see Table 12.)
^{#}Effective allele (EA)-frequency and odds ratio based on this SNP for each locus.

TABLE 15
Association results of some published candidate SNPs not showing
significant evidence of association in TMMG.

| | | | | | | Frequency | | Imputation | | |
|---|---|---|---|---|---|---|---|---|---|---|
| Reference | SNP | GENE | CHR | BP | EA^{#} | Cases | Controls | Quality | OR | P |
| 1 | rs2290465 | VLDLR | 9 | 2645201 | C | 0.817 | 0.81 | 0.515 | 1.08 | 0.252 |
| 1 | rs2010963 | VEGF | 6 | 43738350 | C | 0.341 | 0.338 | 0.899 | 1.05 | 0.258 |
| 1 | rs7294695 | LRP6 | 12 | 12323618 | C | 0.532 | 0.539 | 0.954 | 0.983 | 0.649 |
| 2 | rs3775291 | TLR3 | 4 | 187004074 | T | 0.306 | 0.294 | 0.981 | 1.03 | 0.479 |
| 3 | rs4986790 | TLR4 | 9 | 120475302 | A | 0.938 | 0.939 | 0.911 | 0.963 | 0.634 |
| 4 | rs2511989 | SERPING1 | 11 | 57378325 | T | 0.399 | 0.398 | 1 | 1.02 | 0.648 |
| 5 | rs3732378 | CX3CR1 | 3 | 39307162 | A | 0.172 | 0.168 | 0.905 | 1.02 | 0.715 |
| 5 | rs3793784 | ERCC6 | 10 | 50747539 | C | 0.412 | 0.411 | 0.977 | 0.998 | 0.957 |
| 6 | rs4073 | IL8 | 4 | 74606024 | A | 0.454 | 0.449 | 0.984 | 1.01 | 0.788 |

1. Haines JL, et al. (2006) Functional candidate genes in age-related macular degeneration: significant association with VEGF, VLDLR, and LRP6. Invest Ophthalmol Vis Sci 47: 329-335.
2. Yang Z, et al. (2008) Toll-like receptor 3 and geographic atrophy in age-related macular degeneration. N Engl J Med 359: 1456-1463.
3. Zareparsi S, et al. (2005) Toll-like receptor 4 variant D299G is associated with susceptibility to age-related macular degeneration. Hum Mol Genet 14: 1449-1455.
4. Ennis S, et al. (2008) Association between the SERPING1 gene and age-related macular degeneration: a two-stage case-control study. Lancet 372: 1828-1834.
5. Tuo J, et al. (2004) The involvement of sequence variation and expression of CX3CR1 in the pathogenesis of age-related macular degeneration. FASEB J 18: 1297-1299.
6. Goverdhan SV, et al. (2008) Interleukin-8 promoter polymorphism −251A/T is a risk factor for age-related macular degeneration. Br J Ophthalmol 92: 537-540.
^{#}Effective allele (EA)-frequency and odds ratio based on this SNP for each locus.

Additional methods for marker discovery and validation also were used. Briefly, the Tufts/MGH replication dataset was comprised of DNA samples from unrelated Caucasian individuals not included in the GWAS, including 868 advanced AMD cases and 410 examined controls who were identified from the same Tufts cohorts, and 379 unexamined MGH controls.

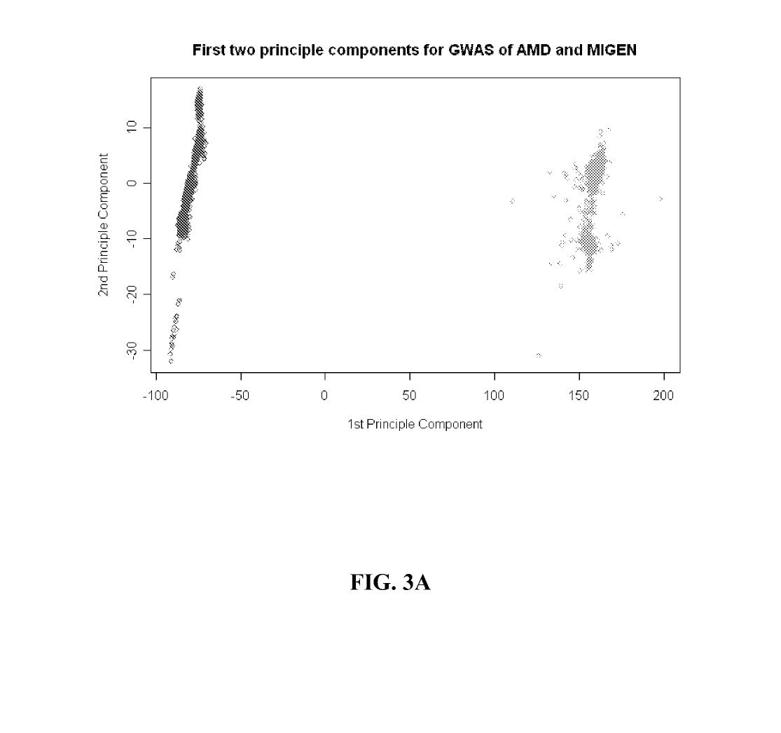

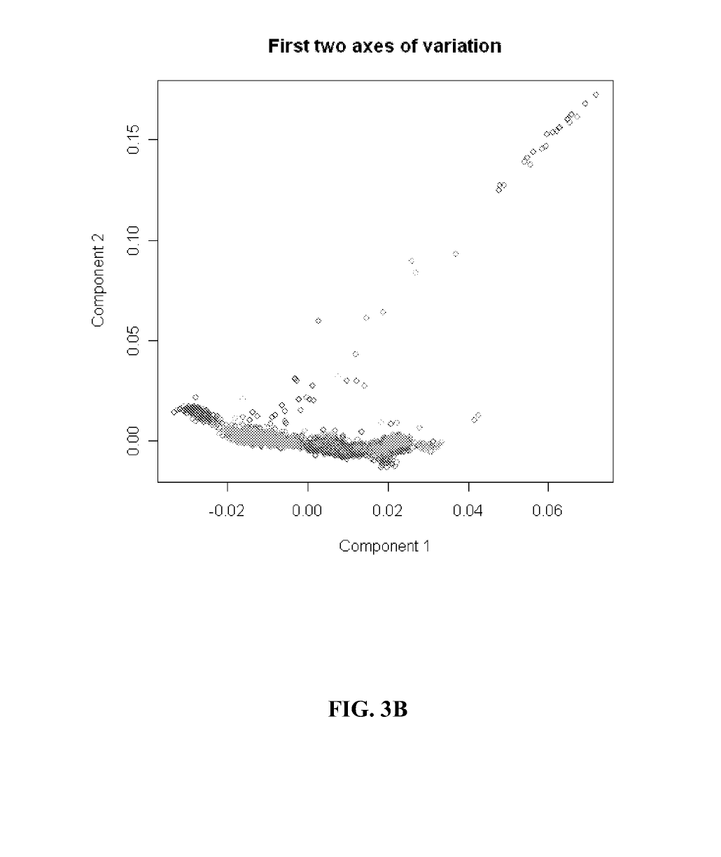

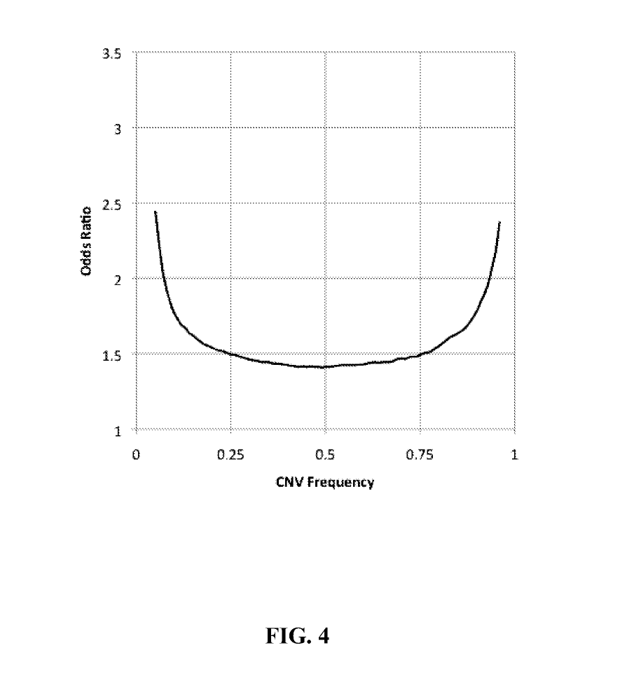

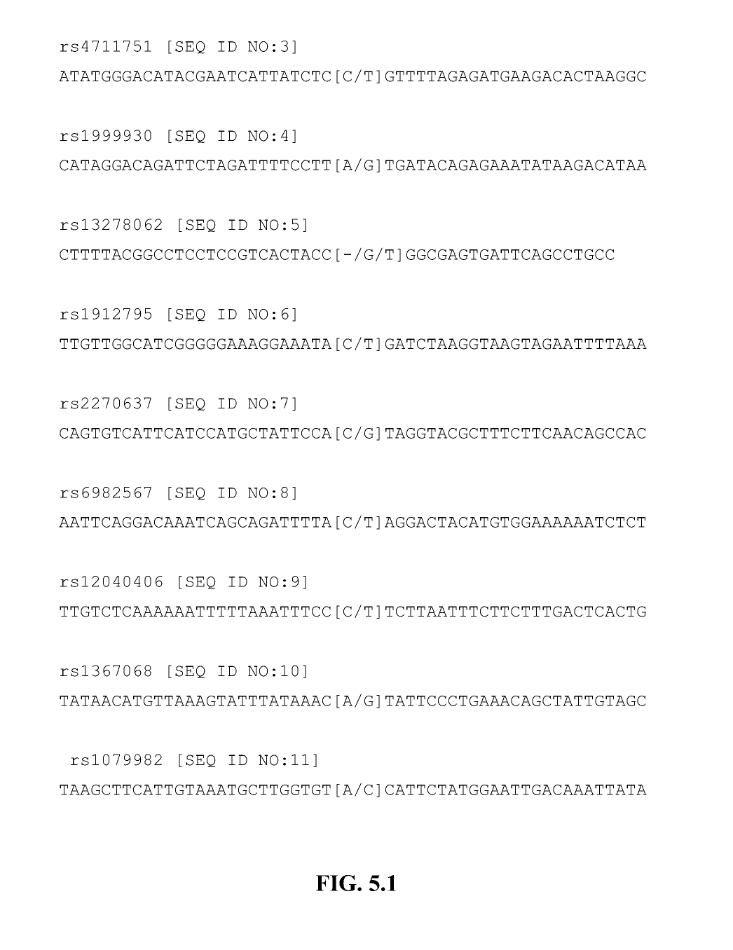

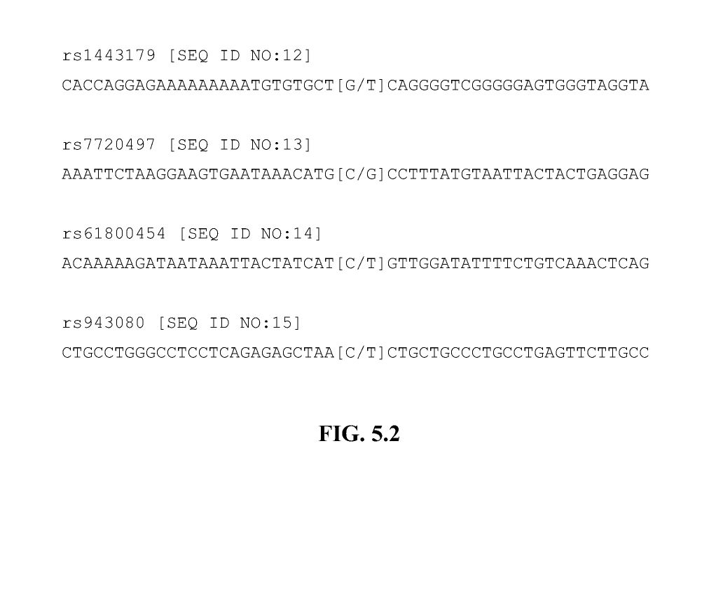

The GWAS genotyping and the Tufts/MGH follow-up replication genotyping were performed at the Broad and National Center for Research Resources (NCRR) Center for Genotyping and Analysis using the Affymetrix SNP 6.0 GeneChip and the Sequenom MassARRAY system for iPLEX assays, respectively. Initially, a primary dataset of 1,057 cases and 558 was examined controls and studied 906,000 genotyped SNPs and 946,000 CNVs using the Affymetrix 6.0 GeneChip which passed quality control filters. Then 43,562 SNPs were removed for low call rate, 4,708 were removed for failing Hardy-Weinberg test at 10-3, and 8,332 SNPs were removed because of failing a differential missing test between cases and controls at 10-3. Finally, 126,050 SNPs were removed for having allele frequency less than 1%, similar to other studies using this methodology. Thus, 726,970 SNPs were evaluated in this study in the discovery phase. 73 individuals were removed for lower than expected call rate, resulting in 1,006 cases and 536 controls. All quality control steps were performed using PLINK. A preliminary χ2 association analysis was conducted to determine the extent to which population stratification and other biases were affecting the samples and observed a lambda of ˜1.05, indicating that the samples were generally well matched for population ancestry, with some minor inflation remaining (explanation and visual representation see FIGS. 3A & 3B). MIGEN shared controls were added, which were genotyped on the same Affymetrix 6.0 GeneChip® product, and population stratification analyses were conducted using multi-dimensional scaling in PLINK. These analyses identified 27 cases, 12 AMD controls and 223 MIGEN controls for a total of 262 individuals which were outliers in the principal component analysis. The final genomic control lambda for the logistic regression included seven significant (for prediction of phenotype status) principal components as covariates and was 1.036 for 632,932 SNPs. This dataset was used for our official GWAS analysis.

SNPs with P<10-3 were evaluated from the GWAS discovery sample (n=720 SNPs excluding previously associated regions) in the MPM GWAS. The exchange of top hits enabled us to use the two scans as primary replication efforts which enhanced the power of each study. Genotyping was performed of all SNPs with combined P<10-4 using Sequenom iPLEX™ at the Broad NCRR Genotyping Center using our Tufts/MGH replication sample. Focusing on sites which continued to show association with P<10-4 after this local replication, a third stage of replication was performed with collaborators in Iceland (deCode Genetics database). For this study, P values were calculated for the combined imputed dataset and for all of our top hits comparing AMD to controls (N=130). Other groups were asked to check these SNPs in their GWAS data—including Iceland (deCode Genetics database) and Genentech—and they sent their data for these SNPs, which were then added to our analyses. To validate the discovery of these SNPs, other groups also were asked to genotype SNPs of interest in their samples using either TaqMan or Sequenom as part of the replication. These data were received as well and combined values were calculated based on the frequencies, of the alleles in the various AMD groups—the total advanced AMD case group, as well as the different advanced phenotypes, called geographic atrophy and neovascular AMD. P values for association between these various alleles, genotypes and different AMD case groups were calculated. SNPs associated with geographic atrophy and neovascular disease were studied and these groups were compared to each other, to determine which are associated with one advanced subtype versus the other. 20 SNPs were identified in this comparison (Table 5), and these SNPs were also sent to the same groups noted above, for replication.

To augment the control set, a subset of controls (n=1409) from the Myocardial Infarction Genetics (MIGEN) Project was used. Briefly, MIGEN controls are ascertained across Europe, for absence of an MI event. These controls are unscreened for AMD, and so the utility of including them was assessed by examining the previously reported associations in the literature. Specifically, an assessment as to whether the loci at CFH, ARMS2, CFI, C3, CF/B2 showed more significant association to AMD upon expansion of the control sample was performed. The inclusion of these shared controls yielded a dramatic increase in the lambda (2.2). Multi-dimensional scaling was applied based on all pair-wise identity-by-state comparisons for all individuals. The first multi-dimensional scaling component separated out completely the shared controls from the initial dataset (FIG. 3A). American populations can be matched to European populations (as long the European populations are diverse), so this complete delineation between the shared controls and the original dataset was due to technical bias between the two datasets. Moving the call rate threshold from 95% to 99% dramatically reduced the lambda (1.22), but still, apparent population stratification effects persisted. Multi-dimensional scaling was again applied to the IBS matrix, examining the first 10 axes of variation. The first axis of variation no longer classified the cases and controls. The second axis of variation identified a handful of individuals who were apparently either demonstrating high levels of technical bias or were from a different ancestral background (FIG. 3B). Finally, the axes of variation were examined to determine whether they significantly predicted case or control status across the genome at an average P-value less than 0.05. Doing so yielded 7 axes of variation and a lambda of 1.036, comparable to the initial study lambda, with an expanded sample size.

TABLE 1
Age-related macular degeneration grade,
gender and age information for samples.

| | Tufts/MGH | UM | Tufts/MGH | | |
|---|---|---|---|---|---|
| | Affy | ILMN | Replication | JHU | NY |
| | N (%) | N (%) | N (%) | N (%) | N (%) |
| AMD | | | | | |
| Grade | | | | | |
| 1 | 524 (35) | 1138 (44) | 410 (32) | 136 (22) | 368 (33) |
| 4 | 269 (18) | 415 (16) | 246 (19) | 95 (15) | 211 (19) |
| 5 | 710 (47) | 1037 (40) | 622 (49) | 389 (63) | 524 (48) |
| Gender | | | | | |
| (N %) | | | | | |
| M | 691 (46) | 1062 (41) | 513 (40) | 209 (34) | 418 (40) |
| F | 812 (54) | 1528 (59) | 765 (60) | 411 (66) | 632 (60) |
| Mean | | | | | |
| Age by | | | | | |
| AMD | | | | | |
| grade | | | | | |
| 1 | 76 | 74 | 73 | 74 | 75 |
| 4 | 81 | 78 | 79 | 76 | 80 |
| 5 | 80 | 80 | 80 | 77 | 79 |

Tufts/MGH Affy represents the genome-wide association scan using the Affymetrix 6.0 platform from Tufts Medical Center, Tufts University School of Medicine, without the MIGEN controls included; Tufts/MGH Replication represents the follow up replication pool at MGH/Tufts; UM ILMN represents the genome-wide association scan using the Illumina 322 platform from the University of Michigan; JHU represents the Johns Hopkins University sample replication, and NY represents the Columbia University sample replication. AMD Grading System: grade 1 represents individuals with no drusen or a few small drusen, 4 represents individuals with central or non-central geographic atrophy (“advanced dry type”), and 5 represents individuals with neovascular disease (“advanced wet type”).

TABLE 2
The evolution of sample size as a function
of the quality control process.

| | | | Change to | |
|---|---|---|---|---|
| | Cases | Controls | Sample Size | #SNPs |
| Initial Sample | 1057 | 558 | — | 909622 |
| Initial Dataset Cleaning | 1006 | 536 | −73 | 726970 |
| Addition of Shared Controls | 1006 | 1944 | 1409 | 707919 |
| Removal of Clustering | 979 | 1709 | −262 | 632932 |
| Outliers | | | | |

Each step represents a cleaning stage. The initial sample represents all samples genotyped. The initial dataset cleaning encompasses HWE, call rate, differential missingness between cases and controls, and minor allele frequency threshold. Adding in shared controls, the call rate and MAF thresholds were reapplied. For the final stage, call rate of 99% was required as was the removal of individuals who did not cluster with the majority of the sample.

### Results

Case and Control Sample Development.

The initial study consisted of 1,057 unrelated cases with geographic atrophy or neovascular AMD, and 558 unrelated controls without AMD who were phenotyped based on clinical examination and ocular photography, and identified from studies of genetic-epidemiology of macular degeneration at Tufts Medical Center. The AMD grade in the worst eye was used in the analyses. All individuals were Caucasian from European ancestry (further details about the original and replication study populations can be found in METHODS and Table 1).

To enhance the power of this study, unrelated control resources that were genotyped on the same platform in the same lab were included, and additional stringent quality control to ensure the technical and population compatibility of these datasets was conducted (METHODS and Table 2). The final genotyped sample consisted of 979 cases and 1,709 controls. Using a logistic regression analysis including population structure covariates, genomic control inflation factors were comparable between the initial, similarly ascertained sample and the expanded sample, suggesting that potential population differences have been controlled appropriately (979 cases to 536 controls lamba=1.051; 979 cases to 1,709 controls lambda=1.036). Because these additional controls were unscreened for AMD status and may include individuals who have or might later develop AMD, their impact on established associations was determined. The most compelling previously reported associated regions in AMD: CFH on chromosome 1, CFI on chromosome 4, BF/C2 on chromosome 6, ARMS2/HTRA1 on chromosome 10, and C3 on chromosome 19 were examined. 159 SNPs that were in LD with the most positively associated variant reported in the literature were examined. Of these, 137 showed an improvement in the χ², with the addition of these controls. The average ratio of the initial study's cleaned χ² to final study's cleaned χ² was 1.82—nearly identical to the expected improvement in χ² based on theoretical power calculations of 1.84. As predicted, the addition of a significant number of unselected controls increased the power of this study substantially.

Genome-Wide Association Discovery Phase.

Using a case-control analysis as implemented in PLINK, no SNPs in regions not already reported as being associated with AMD achieved genome-wide significance of 5×10⁻⁸ as defined by Pe'er et al. Several SNPs of interest in regions without previously reported association with P-values between 10⁻⁴ to 10⁻⁶ were identified in the discovery scan (Tables 3, 4, and 5), including rs4711751 (VEGFA) with p=2.66e-5, and rs6982567 (GDF6) with p=6.14e-6, as discussed in more detail below.

Replication Phases. To evaluate the top results from novel regions identified by the scan, several stages of replication analysis were performed. For all SNPs with p<10⁻³ in the genome-wide association scan, results were obtained from the Michigan, Penn, and Mayo scan, selecting only their advanced cases versus controls, and combined the study results as equally weighted-Z scores given the similar sample sizes. From this combined analysis, SNPs with p<10⁻⁴ or higher in our independent local replication sample of advanced cases and controls from Tufts University School of Medicine and Massachusetts General Hospital (Tufts/MGH) were genotyped, who were unrelated to the individuals in our original scan. Not all SNPs could be imputed perfectly in the Michigan scan, given the different sizes and types of genotyping platforms used (Affymetrix 6.0 with 906,000 SNPS and Illumina with 320,000 SNPS). Therefore, a subset of strongly associated SNPs from the scan alone were selected to be genotyped in the local replication sample. After these steps, a subset of promising SNPs were distributed to collaborators at Iceland (DeCode database) and Genentech, for replication in independent samples. A tally of these P-values for the discovery and local replication stages are presented in Tables 3, 4, and 5. Additional identified SNPs are presented in Tables 6, 7, 8, 9, and 10.

Results of Combined Scan and Replication Analysis.

A SNP on chromosome 6, rs4711751 (VEGFA), showed significant association with p=2.66e-5, and a SNP on chromosome 8, rs6982567 (GDF6), showed significant association with a p=6.14e-6. In addition, a different VEGFA SNP, rs943080, is reportedly in LD with this SNP (paper forthcoming) and is about 1950 by from rs6982567. The genome-wide association study results disclosed herein revealed numerous additional SNPs that are associated with AMD (Tables 3, 4, 5, 6, 7, 8, 9, and 10). The nucleic acid sequence corresponding to each reference SNP (rs) number listed in Tables 3, 4, 5, 6, 7, 8, 9, and 10 is incorporated by references herein.

Thus, rs4711751, rs6982567, a SNP listed in Tables 3, 4, 5, 6, 7, 8, 9, and 10, and/or a marker in linkage disequilibrium with one of these SNPs can be used in accordance with the present invention as markers for AMD etiology, for determining susceptibility to AMD, and for predicting disease progression or severity, and for distinguishing risk of geographic atrophy, the advanced dry type of AMD from the advanced wet form of AMD. In addition, any marker in LD with one of these markers can be used as a surrogate marker for AMD etiology, for determining susceptibility to AMD, and for predicting disease progression or severity.

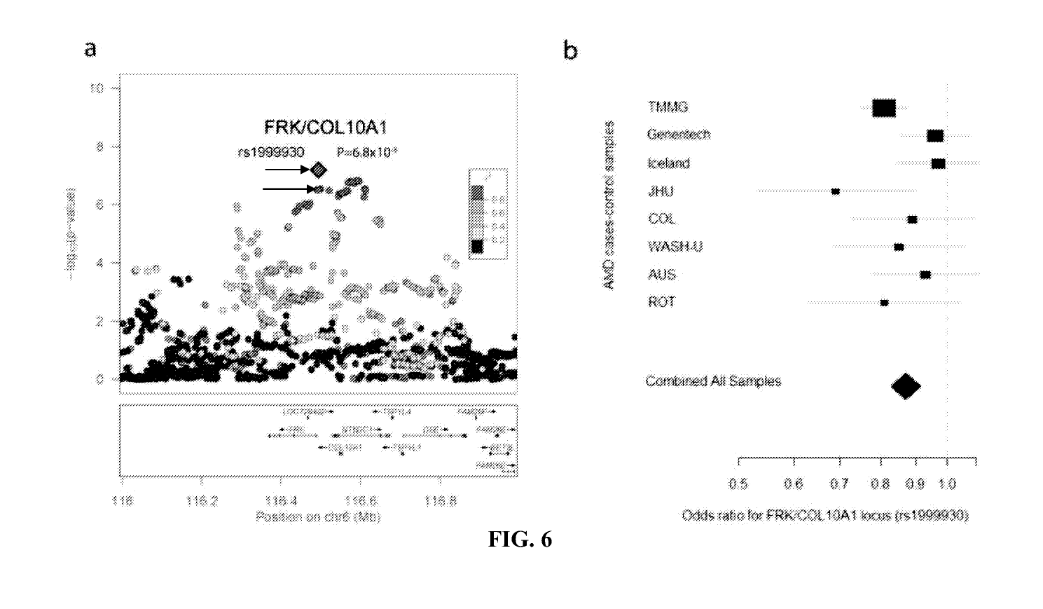

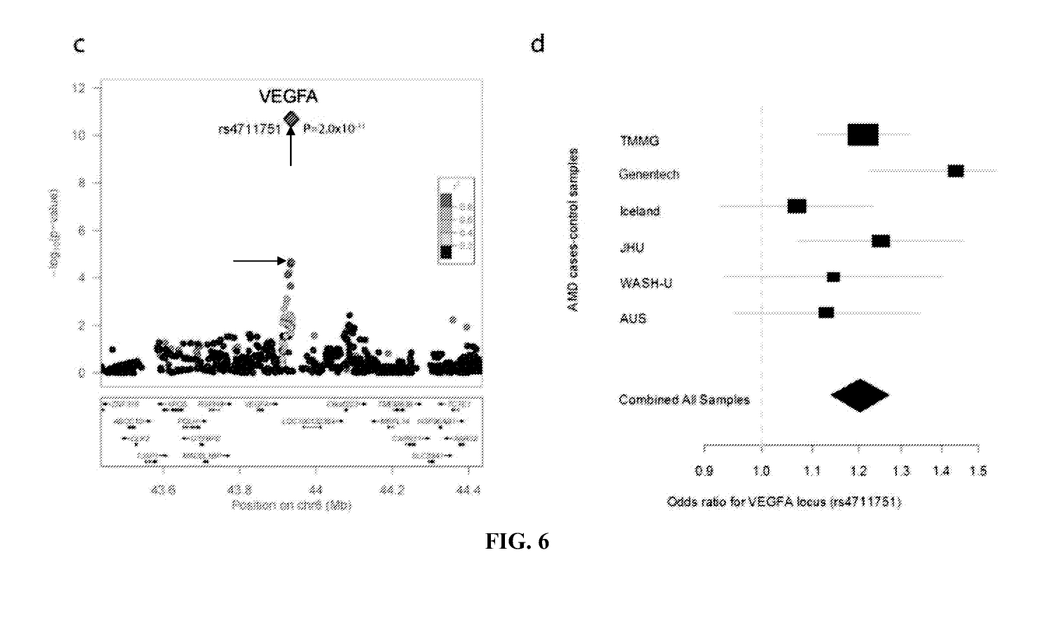

Excluding previously published genetic regions associated with AMD, we detected a region on 6q21-q22.3 (FIG. 6a) containing 30 SNPs with p<5×10⁻⁷ in the TMMG sample. FIGS. 6a-d show the FRK/COL10A1 region and association with AMD. FIG. 6a shows observed association in the 500-kb region surrounding the FRK/COL10A1 locus in meta-analysis of TMMG datasets. The represented SNP (rs1999930) for this region of P=3.4×10⁻⁷ was shown by small purple diamond (see arrow). In the combined analysis including all 8 cohorts this SNP was associated with AMD at P=6.8×10⁻⁸ (large purple diamond; see arrow). FIG. 6b shows Forest plot for rs1999930 association across 8 Cohorts. FIG. 6c shows observed association in the 500-kb region surrounding the VEGFA (rs4711751) locus in meta-analysis of TMMG datasets. In the combined analysis including all 6 cohorts this SNP was associated with AMD at P=2.0×10⁻¹¹ (large purple diamond; see arrow). FIG. 6d shows Forest plot for rs4711751 association across 6 cohorts.

Since all of these SNPs are in a tight LD block (r²>0.8), we chose to investigate the association in this region through rs1999930. The minor T allele frequency of rs1999930 was 26.0% in cases and 30.5% in controls (Table 11) for the TMMG sample, with an odds ratio (OR) of 0.81, and 95% confidence interval (CI) 0.77-0.84. To confirm this new locus for AMD, we tested rs1999930 in a total of 4269 independent cases and 50,938 independent controls of European ancestry from Johns Hopkins University (JHU), Columbia University (COL), Genentech, Decode, Washington University (Wash-U), Centre for Eye Research Australia (AUS), Rotterdam (ROT), and Hopital Intercommunal de Creteil (FR-CRET) (Table 12). Frequency and risk associated with the minor allele T of rs1999930 in each replication cohort were all in the same direction as in TMMG (FIG. 6b).

Combining the test statistics of all independent replication cohorts weighted by their sample size using Stouffer's Z-score method, this association was confirmed (OR=0.91, P=0.0057). The results were very consistent across datasets with no significant evidence for heterogeneity under the Cochran's Q-test for our samples (Q=0.09, I²=44[0-75]). In the combined analysis of all the samples, the T allele of rs1999930 significantly (p=6.8×10⁻⁸) reduced the risk of AMD (OR=0.87 [0.83-0.92]). This associated region represented by rs1999930 contains the genes COL10A1 (encoding the alpha chain of type X collagen) and FRK (encoding fyn-related kinase).

We also tested other unreported loci for AMD with p-value<5×10⁻⁵ in the TMMG meta-analysis (Table 14) and several previously reported loci (Table 11) with suggestive association results. The risk variants in TIMP3 (rs9621532, p=6×10⁻¹⁴) and HDL pathway genes LIPC (rs10468017, p=5.3×10⁻⁹), CETP (rs3764261, p=9.6×10⁻⁹) were genome-wide significant in our combined analysis and a previously suggestive association in ABCA1 (rs1883025, p=9.5×10⁻⁷) was still noteworthy. Another locus near C4BPA/CD55 gene was suggested from the TMMG analysis (P=4.6×10⁻⁷), however, the combined p-value was 9.9×10⁻⁵.

Among the other previously unreported loci, the T allele of one candidate SNP (rs4711751) near VEGFA, was associated with increased risk of AMD (OR=1.21 [1.16-1.27], p=1.5×10⁻⁵) in the TMMG meta-analysis and the results were very consistent in direct genotyping replication in 3277 cases and 42091 controls (OR=1.20 (1.15-1.24), p=2.9×10⁻⁷). This SNP reached genome-wide significance (OR=1.20 [1.17-1.24], p=2.0×10¹¹) in the combined analysis including all replication cohorts (FIGS. 6c, 6d). This novel association with VEGFA was also found in a parallel meta-analysis on a SNP (rs943080, R²=1, D′=1) in LD with rs4711751. Our newly identified SNPs is 3′ downstream of VEGFA and more than 90 kb away from the SNP in VEGFA promoter region (rs2010963), which was reported to be associated with AMD previously (Table 15). The rs2010963 allele is in very low LD with rs4711751 (R²=0.015, D′=0.138); therefore the association we identified in VEGFA was in a novel region and not likely due to LD with SNPs in the VEGFA promoter region. Of note, the previously reported rs2010963 SNP showed little evidence of association in our TMMG meta-analysis (p=0.26).

VEGFA which is a member of the vascular endothelial growth factor family increases vascular permeability, angiogenesis, cell growth and migration of endothelial cells. VEGFA has been a major candidate for AMD risk and it has been hypothesized that activation of this gene may induce pathologic angiogenesis under the retinal epithelial (RPE). Interestingly, Rajpar et al. described creating a knock-in mouse for COL10A1 p.Asn617Lys (possible human SNP rs61745148) which reduced the VEGF expression in hypertrophic chondrocytes leading to a significant reduction in the recruitment of osteoclasts to the vascular invasion front. (Rajpar, M. H., et al., “Targeted induction of endoplasmic reticulum stress induces cartilage pathology.” PLoS Genet 5, e1000691 (2009))

Furthermore, hypoxia-inducible factor-2α (HIF-2α, encoded by EPAS1) was shown to enhance promoter activities of COL10A1, MMP13 and VEGFA through specific binding to the respective hypoxia-responsive elements. (Saito, T. et al., “Transcriptional regulation of endochondral ossification by HIF-2alpha during skeletal growth and osteoarthritis development.” Nat Med 16, 678-86 (2010)) Hypoxia is known to increase VEGF transcription, translation, and mRNA stability because VEGFA is extremely sensitive to oxygen levels. VEGFA signaling of the Akt pathway can be antagonized by transpondin-1 (TSP-1) which can modulate the remodeling of the microvascular network of the developing retina. FRK has been shown to have negative function on the stimulation of microvascular survival by mediating the downstream signaling of TSP1 and the TSP receptor (CD36). (Sun, J. et al., “Thrombospondin-1 modulates VEGF-A-mediated Akt signaling and capillary survival in the developing retina.” Am J Physiol Heart Circ Physiol 296, H1344-51 (2009)) It is quite possible that this SNP or set of SNPs in the region directly affect the expression of VEGF through either or both COL10A1 and FRK signaling.

We also investigated the specific association with geographic atrophy (GA) and neovascular (NV) subtypes of AMD in our TMMG samples respectively. Association signals on CFH, C2, CFB, C3, CFI and ARMS2/HTRA1 were also highly significant for both GA and NV compared to controls. The minor allele (T) of rs1999930 had a similar effect size for GA (OR=0.78 [0.69-0.89], P=1.0×10⁻⁴) and NV (OR=0.82 [0.75-0.90], P=4.1×10⁻⁵). The risk allele (T) of rs4711751 also had a similar magnitude of effect on GA (OR=1.23 [1.08-1.40], P=2.0×10⁻³) and NV (OR=1.20 [1.09-1.32], P=2.5×10⁻⁴).

We found two novel associated loci near FRK/COL10A1 and VEGFA, and confirmed associations for ten previously published AMD loci in our combined analysis. The genetic loci associated with AMD suggest that the disease process may be explained in part by pathological activation of the alternative complement pathway (CFH, C2, CFB, C3, CFI), the imbalance of HDL cholesterol metabolism (LIPC, CETP, ABCA1) and possibly angiogenesis (VEGFA) induced by dysfunction or degradation of extracellular matrix (COL10A1, FRK, ARMS2, TIMP3).

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

TABLE 3

| | | | | P_1vs45_{—} | |
|---|---|---|---|---|---|
| SNP | GENE | EA | OR(EA) | Tufts_MPM | Meta_P |
| rs4711751 | VEGFA | T | 1.22 | 2.7E−05 | 4.3E−11 |
| rs6982567 | GDF6 | T | 1.32 | 6.1E−06 | 3.6E−07 |
| rs9366769 | HCG27(0) | C | 0.80 | 9.4E−06 | 6.8E−07 |
| rs1999930 | FRK | T | 0.81 | 2.2E−06 | 1.6E−06 |
| rs2270637 | SLC18A1 | G | 0.81 | 5.4E−05 | 3.8E−06 |
| rs13278062 | TNFRSF10A(+0.332 kb)\|CHMP7(−18.18 | T | 1.22 | 5.0E−05 | 3.8E−06 |
| | kb) | | | | |
| rs7720497 | ADAMTS16 | G | 1.45 | 2.1E−05 | 5.8E−06 |
| chr1:163986967 | TMCO1(0) | T | 0.57 | 5.4E−05 | 7.8E−06 |
| rs1443179 | INTU | T | 1.66 | 1.4E−06 | 8.9E−06 |
| rs513683 | P4HA3(0) | C | 0.85 | 7.6E−05 | 1.4E−05 |
| rs7626245 | FILIP1L(0)\|C3orf26(0) | C | 1.36 | 6.6E−05 | 1.6E−05 |
| rs12632105 | RHO(−139 kb)\|MBD4(−41.36 kb)\|IFT122(−50.53 | C | 1.25 | 8.1E−05 | 2.0E−05 |
| | kb)\|H1FX(+73.32 kb)\|COPG(−111.8 kb) | | | | |
| rs2052572 | TSHZ3(+107.3 kb) | A | 1.19 | 4.4E−05 | 2.3E−05 |
| rs6587759 | ZNF692(+10.26 kb)\|SH3BP5L(+43.38 kb) | A | 1.44 | 4.2E−05 | 3.6E−05 |
| | \|PGBD2(−36.91 kb)\| | | | | |
| chr22:33891064 | ISX(+77.68 kb)\|HMG2L1(−92.42 kb) | T | 0.51 | 9.9E−06 | 4.3E−05 |
| rs16965939 | TMCO5 | T | 0.64 | 1.1E−06 | 4.5E−05 |
| rs3760775 | FUT3 | T | 0.66 | 1.3E−05 | 5.1E−05 |
| chr12:8919954 | A2ML1(0) | T | 0.48 | 4.7E−05 | 5.4E−05 |
| rs10103808 | FAM135B(0) | T | 1.34 | 1.9E−05 | 7.1E−05 |
| chr2:180796214 | KIAA1604(+216.2 kb) | A | 0.81 | 3.9E−05 | 8.6E−05 |
| rs2883171 | CDH12(−477.3 kb) | G | 1.29 | 7.2E−05 | 8.7E−05 |
| rs2037156 | EPHA7(−434.6 kb) | G | 1.27 | 7.9E−06 | 1.1E−04 |
| rs6531212 | WDR35(+148.1 kb)\|TTC32(+236.3 kb)\|S | T | 0.83 | 2.1E−05 | 1.3E−04 |
| | DC1(−62.54 kb)\|PUM2(−110.4 | | | | |
| | kb)\|MATN3(+125.6 kb)\|LAPTM4A(+86.58 kb) | | | | |
| rs2798832 | TAF3(+129.4 kb)\|KIN(+356.1 kb)\|ITIH2(+394.6 | T | 1.22 | 5.5E−05 | 1.3E−04 |
| | kb)\|ITIH5(+477.2 kb)\|GATA3(+68.93 | | | | |
| | kb)\|FLJ45983(+90.65 kb)\|ATP5C1(+336.3 kb) | | | | |
| rs8053796 | CNTNAP4 | T | 1.27 | 4.4E−06 | 1.6E−04 |
| rs1851808 | MRPL19(+489.6 kb) | G | 1.20 | 2.7E−05 | 2.2E−04 |
| rs10067691 | SDCCAG10(0) | A | 0.67 | 1.7E−05 | 2.4E−04 |
| chr10:57479107 | ZWINT(−308.1 kb) | A | 1.29 | 8.3E−05 | 2.4E−04 |
| rs728371 | MOXD1(−134.9 kb)\|CTGF(+209.8 kb) | A | 1.23 | 4.7E−06 | 2.4E−04 |
| rs4967980 | USP31(+23 kb)\|UBFD1(−385.3 | C | 1.23 | 3.9E−05 | 2.5E−04 |
| | kb)\|SCNN1B(−130 kb)\|SCNN1G(−10.45 | | | | |
| | kb)\|COG7(−216.2 kb) | | | | |
| rs1540819 | LSAMP(+563.4 kb) | T | 1.17 | 9.7E−05 | 2.8E−04 |
| rs529478 | NEDD4L(0) | T | 0.83 | 3.3E−05 | 2.8E−04 |
| rs1915098 | IGSF11(−421 kb) | A | 0.78 | 2.7E−06 | 2.9E−04 |
| chr4:86206508 | WDFY3(+99.94 kb) | T | 0.61 | 6.9E−05 | 3.0E−04 |
| chr5:113645446 | KCNN2(−80.47 kb) | G | 1.29 | 5.5E−05 | 3.2E−04 |
| chr11:124212812 | ROBO3(−27.68 kb)\|C11orf61(+37.3 kb) | T | 1.60 | 6.7E−05 | 3.5E−04 |
| rs12520598 | GCNT4(+22.83 kb)\|HMGCR(−283.5 kb) | T | 0.70 | 4.2E−05 | 3.5E−04 |
| rs11067403 | TBX3(+404 kb) | C | 0.84 | 5.7E−05 | 3.8E−04 |
| chr5:149749881 | TCOF1(0) | C | 1.38 | 9.2E−05 | 3.9E−04 |
| rs1079982 | ANKRD54, CARD10, CARMA3, CDC42E | T | 0.65 | 3.0E−06 | 4.0E−04 |
| | P1, FLJ00017, GALR3, GCAT, GGA1, H1F | | | | |
| | 0, LGALS1, LGALS2, LRRC62, MFNG, NO | | | | |
| | L12, PDXP, PSCD4, RAC2, SH3BP1, TRIO | | | | |
| | BP | | | | |
| rs12908430 | GANC(0) | A | 0.71 | 5.3E−05 | 4.2E−04 |
| rs4256145 | OTOL1(+488.4 kb) | G | 0.84 | 3.6E−05 | 4.7E−04 |
| chr8:123521912 | ZHX2(−341.2 kb) | A | 1.78 | 3.6E−05 | 4.7E−04 |
| rs9328048 | IRF4(−675.1 kb) | T | 0.78 | 2.3E−06 | 4.9E−04 |
| chr1:59476907 | FGGY(−58.3 kb) | A | 0.57 | 9.9E−05 | 5.6E−04 |
| rs7783337 | DGKB | G | 1.27 | 8.8E−05 | 5.7E−04 |
| chr15:35201758 | MEIS2(+20.97 kb) | T | 1.39 | 8.8E−05 | 6.4E−04 |
| chr1:85757359 | DDAH1(+53.95 kb)\|CYR61(−61.69 kb) | T | 0.70 | 8.3E−05 | 6.5E−04 |
| rs2491146 | SDC3(−13.09 kb)\|PUM1(−75.13 | A | 0.82 | 2.4E−05 | 6.6E−04 |
| | kb)\|MATN1(+132.3 kb)\|LAPTM5(+98.56 kb) | | | | |
| chr4:182189551 | ODZ3(−1293 kb) | G | 1.74 | 2.6E−06 | 6.8E−04 |
| chr1:100166675 | AGL(+4.508 kb)\|SLC35A3(−41.45 kb) | T | 1.50 | 1.8E−05 | 7.0E−04 |
| rs11854658 | MFGE8(+54.89 kb)\|HAPLN3(+72.78 kb)\| | G | 0.82 | 3.9E−06 | 7.6E−04 |
| | ACAN(+92.97 kb)\|ABHD2(−119.9 kb) | | | | |
| rs1912795 | B3GALTL(0) | A | 1.19 | 3.2E−05 | 8.6E−04 |
| chr19:14775139 | OR7C1(+3.191 kb)\|OR7A5(−23 kb) | A | 0.71 | 6.6E−05 | 8.7E−04 |
| chr11:1768072 | CTSD(−231.9 kb) | A | 0.75 | 4.8E−06 | 9.1E−04 |
| rs7958529 | ACVR1B, ACVRL1, bpl_41- | G | 0.80 | 5.5E−05 | 9.5E−04 |
| | 16, C12orf44, GRASP, KRT6A, KRT6B, KR | | | | |
| | T6C, KRT7, KRT75, KRT80, KRT81, KRT8 | | | | |
| | 2, KRT83, KRT84, KRT85, KRT86, NR4A1 | | | | |
| rs17071512 | NDFIP2(0) | C | 0.71 | 1.1E−05 | 9.7E−04 |
| rs4260755 | HMGCLL1(0) | A | 1.20 | 7.5E−05 | 9.8E−04 |
| rs2024393 | ZC3H7A(+30.67 kb)\|TXNDC11(+70.43 kb) | T | 1.53 | 7.5E−05 | 1.0E−03 |
| | \|TNFRSF17(−151.9 | | | | |
| | kb)\|SNN(+134.1 kb)\|RUNDC2A(−163.5 | | | | |
| | kb)\|RSL1D1(−20.98 kb)\|GSPT1(−54.91 kb) | | | | |
| rs12632671 | TKT | A | 1.50 | 5.7E−05 | 1.1E−03 |
| rs13253938 | ZFAT(+315.9 kb)\|KHDRBS3(−428.6 kb) | G | 0.83 | 3.0E−05 | 1.1E−03 |
| rs343718 | TBX20(+335.4 kb)\|SEPT7(−212 | A | 0.84 | 5.7E−05 | 1.1E−03 |
| | kb)\|HERPUD2(−43.64 kb) | | | | |
| rs1384044 | SGGZ(−276.3 kb)\|DLC1(+298.6 kb) | T | 0.84 | 1.7E−05 | 1.1E−03 |
| chr4:23188024 | PPARGC1A(−214.7 kb) | A | 0.78 | 5.3E−05 | 1.2E−03 |
| rs722782 | MYOM2(−1494 kb) | A | 0.76 | 5.2E−05 | 1.4E−03 |
| rs1737478 | CREG1 | T | 1.21 | 8.5E−05 | 1.4E−03 |
| rs5771717 | FAM19A5 | A | 1.36 | 9.5E−06 | 1.5E−03 |
| rs164700 | EFNA5(0) | A | 1.27 | 9.3E−05 | 1.5E−03 |
| rs7623235 | DKFZp667G2110(0) | A | 1.26 | 5.2E−05 | 1.6E−03 |
| rs6685751 | SPHAR(−384.2 kb)\|RHOU(+173.5 kb)\|RAB4A(−350.9 | A | 1.27 | 6.0E−05 | 1.7E−03 |
| | kb) | | | | |
| rs1358395 | GRM3(−34.61 kb) | A | 0.84 | 9.4E−05 | 1.7E−03 |
| chr10:88344492 | WAPAL(+72.97 kb)\|OPN4(−59.8 kb) | A | 1.29 | 2.0E−05 | 1.7E−03 |
| rs11183802 | FAM113B | A | 1.45 | 4.2E−06 | 1.8E−03 |
| chr3:116361297 | ZBTB20(+12.48 kb) | T | 0.73 | 1.1E−05 | 1.9E−03 |
| rs5015852 | ENPP3(0) | T | 0.58 | 6.5E−05 | 2.2E−03 |
| rs10848645 | CACNA1C(0) | G | 1.19 | 9.4E−05 | 2.2E−03 |
| chr10:56380194 | PCDH15(+149.1 kb) | G | 1.68 | 4.3E−05 | 2.3E−03 |
| rs28621471 | MAPK10 | A | 0.76 | 3.7E−05 | 2.5E−03 |
| chr8:40099493 | INDO(+194.4 kb) | T | 1.63 | 8.9E−05 | 2.7E−03 |
| rs16822447 | RAP2B(+65.39 kb) | T | 0.78 | 9.0E−05 | 2.7E−03 |
| rs9291737 | FLJ37543(+74.42 kb) | T | 0.84 | 1.3E−05 | 2.7E−03 |
| chr2:44132778 | PPM1B(−116.7 kb)\|LRPPRC(+56.13 kb) | G | 0.71 | 5.5E−05 | 2.9E−03 |
| rs2376241 | RELL1(−428.5 kb)\|C4orf19(−291.6 kb) | T | 1.20 | 8.0E−05 | 3.0E−03 |
| rs10225927 | VSTM2A(+132.5 kb)\|SEC61G(−50.53 | G | 1.22 | 9.1E−05 | 3.1E−03 |
| | kb)\|EGFR(−317.3 kb) | | | | |
| rs8011890 | TCL1B | A | 1.41 | 2.3E−05 | 3.1E−03 |
| rs6679773 | USH2A(+36.74 kb)\|TGFB2(−1886 | T | 2.75 | 5.3E−05 | 3.4E−03 |
| | kb)\|SPATA17(−1171 kb)\|RRP15(−1825 | | | | |
| | kb)\|PTPN14(+1909 kb)\|KCTD3(+838.3 | | | | |
| | kb)\|KCNK2(+1223 kb)\|GPATCH2(−970.4 | | | | |
| | kb)\|ESRRG(−43.11 | | | | |
| | kb)\|CENPF(+1796 kb) | | | | |
| rs13192030 | SESN1(0) | T | 0.62 | 7.8E−05 | 3.4E−03 |
| chr11:104185708 | CASP4(−133.1 kb) | T | 2.19 | 2.7E−05 | 4.2E−03 |
| rs1367068 | C4BPAL1, C4BPA, CD55 | G | 0.74 | 5.6E−07 | 4.5E−03 |
| rs1867348 | IGF2R(0) | T | 1.34 | 5.2E−05 | 4.5E−03 |
| rs8076470 | SPATA20 | T | 1.21 | 4.0E−06 | 4.9E−03 |
| rs251525 | NDUFS4(−271 kb)\|MOCS2(+179.9 | G | 1.19 | 8.1E−05 | 5.2E−03 |
| | kb)\|ITGA2(+194.9 | | | | |
| | kb)\|ITGA1(+336 kb)\|FST(−191.1 kb) | | | | |
| rs17408651 | ELTD1(+115.3 kb) | T | 0.69 | 7.5E−05 | 5.5E−03 |
| rs1360751 | RREB1(0) | A | 1.18 | 7.4E−05 | 6.0E−03 |
| chr5:165133520 | ODZ2(−1511 kb) | C | 0.78 | 1.0E−05 | 6.1E−03 |
| rs8091635 | BRUNOL4(+711.5 kb) | T | 1.25 | 8.1E−06 | 6.1E−03 |
| chr6:162467903 | PARK2(0) | A | 0.76 | 8.9E−05 | 6.7E−03 |
| rs7260457 | LILRB3 | C | 0.61 | 2.1E−05 | 7.3E−03 |
| chr15:47957127 | ATP8B4(0) | T | 0.67 | 3.8E−05 | 7.4E−03 |
| rs13094238 | MME(−18.73 kb) | C | 0.68 | 1.4E−05 | 7.4E−03 |
| rs12798294 | PDGFD(0) | C | 0.83 | 3.5E−05 | 8.0E−03 |
| rs9308649 | CNTNAP5(0) | T | 0.81 | 1.7E−05 | 8.2E−03 |
| rs10503493 | SGCZ(0) | A | 0.68 | 6.8E−05 | 8.2E−03 |
| rs9461856 | SYNGAP1(0) | A | 0.85 | 5.3E−05 | 8.4E−03 |
| rs11592003 | KLF6(+127.8 kb) | C | 0.79 | 4.8E−05 | 9.1E−03 |
| chr12:11233234 | TAS2R42(+2.424 kb) | A | 1.29 | 2.1E−05 | 9.3E−03 |
| rs10768315 | RAG2(+1021 kb) | T | 0.81 | 2.0E−05 | 1.0E−02 |
| rs2730613 | MRPS24 | C | 0.83 | 1.5E−05 | 1.2E−02 |
| rs10280782 | TRA2A, IGF2BP3, CLK2P | G | 0.75 | 2.6E−06 | 1.3E−02 |
| rs7094579 | RPP30(−238.9 kb)\|HTR7(−107.8 | T | 0.70 | 8.4E−05 | 1.3E−02 |
| | kb)\|ANKRD1(−279 kb) | | | | |
| chr5:26212150 | CDH9(−704.3 kb) | C | 0.70 | 3.9E−05 | 1.4E−02 |
| rs2135548 | FOXP1(0) | G | 1.20 | 9.4E−05 | 1.5E−02 |
| rs625761 | TMEM133(−218.2 kb)\|PGR(−255.8 | G | 0.82 | 9.7E−05 | 1.5E−02 |
| | kb)∥CNTN5(+417.1 kb) | | | | |
| rs1418473 | DAB1(−16.87 | A | 1.57 | 3.0E−06 | 1.6E−02 |
| | kb)\|C8B(+15.02 kb)\|C8A(+62.82 kb) | | | | |
| rs2277252 | ARHGAP12(−98.91 kb) | A | 1.23 | 6.4E−06 | 2.6E−02 |
| rs4073997 | NPLOC4(0) | C | 1.26 | 9.8E−05 | 3.0E−02 |
| chr17:72483712 | MGAT5B(+25.65 kb) | G | 0.63 | 5.3E−05 | 3.1E−02 |
| rs1492116 | AEBP2(+312 kb) | C | 0.82 | 6.4E−05 | 3.4E−02 |
| rs9713311 | CDGAP(0) | C | 1.18 | 5.9E−05 | 3.5E−02 |
| rs2127740 | PSMD7(−598.4 kb) | A | 1.29 | 3.3E−05 | 3.9E−02 |
| chr6:119117772 | PLN(+129.5 kb)\|MCM9(−155.7 | T | 0.71 | 4.0E−05 | 4.0E−02 |
| | kb)\|ASF1A(−145.9 kb) | | | | |
| rs1789110 | MBP | C | 1.22 | 2.3E−06 | 4.7E−02 |
| chr16:17310751 | XYLT1(0) | G | 0.72 | 2.7E−05 | 4.7E−02 |
| rs235195 | TUBB1(−48.54 kb)\|TH1L(−10.54 | A | 1.37 | 5.1E−05 | 7.5E−02 |
| | kb)\|STX16(+291.2 kb)\|SLMO2(−62.43 | | | | |
| | kb)\|NPEPL1(+254.9 kb)\|GNAS(+59.52 | | | | |
| | kb)\|CTSZ(−24.47 kb)\|ATP5E(−57.97 kb) | | | | |
| rs11596472 | ARL5B(+385.1 kb) | G | 1.42 | 6.1E−06 | 8.7E−02 |
| rs12926103 | FOXF1(−172.4 kb) | A | 1.41 | 2.7E−05 | 1.5E−01 |
| rs17155281 | HIBADH(−56.23 kb) | C | 0.79 | 3.3E−05 | 1.9E−01 |
| rs135912 | PRR5(−270.7 kb)\|PARVB(+276.2 kb)\|PARVG(+238.7 | G | 0.77 | 9.1E−05 | 2.2E−01 |
| | kb)\|LDOC1L(−94.52 | | | | |
| | kb)\|KIAA1644(+132.5 kb) | | | | |
| chr3:126793536 | OSBPL11(0) | C | 0.53 | 9.6E−05 | 2.7E−01 |
| rs16971055 | TNRC6C(+225.5 kb)\|TMC8(+187.5 kb)\|T | A | 1.55 | 2.8E−05 | 2.9E−01 |
| | MC6(+198.1 kb)\|TK1(+143.3 kb)\|SYNGR | | | | |
| | 2(+157.5 kb)\|SOCS3(−26.32 kb)\|PGS1(−48.19 | | | | |
| | kb)\|BIRC5(+104.8 kb)\|AFMID(+122.8 kb) | | | | |

TABLE 4

| IMP | SNP | CHR:BP | A1 | A2 | FRQ_1 | FRQ_45 | FRQ_4 | FRQ_5 | P_1vs45 |
|---|---|---|---|---|---|---|---|---|---|
| 1KG | rs1367068 | 1:205461564 | A | G | 0.7715 | 0.8076 | 0.8035 | 0.8101 | 5.60E−07 |
| 1KG | rs1443179 | 4:128495772 | T | G | 0.0587 | 0.077 | 0.0856 | 0.0732 | 1.44E−06 |
| 1KG | rs9328048 | 6:1324870 | T | C | 0.3985 | 0.3608 | 0.3645 | 0.3591 | 2.26E−06 |
| 1KG | chr4: 182189551 | 4:182189551 | A | G | 0.894 | 0.8848 | 0.8894 | 0.882 | 2.58E−06 |
| 1KG | rs1915098 | 3:119681130 | A | G | 0.2202 | 0.1857 | 0.1744 | 0.192 | 2.74E−06 |
| 1KG | rs1418473 | 1:57219300 | A | G | 0.0511 | 0.0785 | 0.0873 | 0.074 | 3.03E−06 |
| 1KG | rs11183802 | 12:45893566 | A | G | 0.0929 | 0.1221 | 0.1099 | 0.1276 | 4.17E−06 |
| 1KG | rs728371 | 6:132523980 | A | G | 0.3117 | 0.3509 | 0.3601 | 0.3469 | 4.69E−06 |
| 1KG | chr11: 1768072 | 11:1768072 | A | G | 0.1756 | 0.1443 | 0.1485 | 0.1425 | 4.81E−06 |
| 1KG | rs11596472 | 10:19392088 | A | G | 0.8158 | 0.7885 | 0.778 | 0.7939 | 6.06E−06 |
| 1KG | rs6982567 | 8:96819457 | T | C | 0.1321 | 0.1714 | 0.1582 | 0.1778 | 6.14E−06 |
| 1KG | rs2277252 | 10:32036318 | A | G | 0.4581 | 0.4968 | 0.4978 | 0.4974 | 6.45E−06 |
| 1KG | rs8091635 | 18:34111545 | T | C | 0.3605 | 0.4065 | 0.3814 | 0.4189 | 8.08E−06 |
| 1KG | rs9366769 | 6:31277268 | T | C | 0.7696 | 0.8052 | 0.81 | 0.8026 | 9.42E−06 |
| 1KG | rs5771717 | 22:47467676 | A | G | 0.0904 | 0.1136 | 0.1473 | 0.0989 | 9.52E−06 |
| 1KG | chr22: 33891064 | 22:33891064 | T | C | 0.0767 | 0.0618 | 0.0651 | 0.0602 | 9.91E−06 |
| 1KG | chr5: 165133520 | 5:165133520 | T | C | 0.8074 | 0.8402 | 0.8363 | 0.8429 | 1.02E−05 |
| 1KG | chr3: 116361297 | 3:116361297 | T | C | 0.197 | 0.1745 | 0.1779 | 0.1734 | 1.07E−05 |
| 1KG | rs17071512 | 13:78960018 | T | C | 0.877 | 0.8999 | 0.8931 | 0.9027 | 1.08E−05 |
| 1KG | rs1384044 | 8:13715406 | T | C | 0.53 | 0.4926 | 0.4865 | 0.4962 | 1.66E−05 |
| 1KG | rs9308649 | 2:124751486 | A | T | 0.7856 | 0.8183 | 0.827 | 0.8143 | 1.71E−05 |
| 1KG | rs10067691 | 5:64292165 | A | G | 0.1102 | 0.0917 | 0.095 | 0.0897 | 1.72E−05 |
| 1KG | chr1: 100166675 | 1:100166675 | T | C | 0.0469 | 0.061 | 0.0666 | 0.0585 | 1.81E−05 |
| 1KG | rs10768315 | 11:37597461 | T | C | 0.2262 | 0.2126 | 0.2041 | 0.2166 | 2.00E−05 |
| 1KG | chr10: 88344492 | 10:88344492 | A | C | 0.1391 | 0.163 | 0.1577 | 0.1659 | 2.03E−05 |
| 1KG | rs7260457 | 19:59425996 | C | G | 0.2151 | 0.1969 | 0.1929 | 0.1986 | 2.07E−05 |
| 1KG | chr12: 11233234 | 12:11233234 | A | T | 0.2622 | 0.2941 | 0.2924 | 0.2934 | 2.14E−05 |
| 1KG | rs2491146 | 1:31101812 | A | G | 0.4538 | 0.4187 | 0.4062 | 0.4238 | 2.38E−05 |
| 1KG | chr16: 17310751 | 16:17310751 | A | G | 0.8922 | 0.9123 | 0.9132 | 0.9114 | 2.66E−05 |
| 1KG | rs4711751 | 6:43936560 | T | C | 0.5164 | 0.5475 | 0.5531 | 0.5445 | 2.66E−05 |
| 1KG | chr11: 104185708 | 11:104185708 | T | G | 0.0696 | 0.076 | 0.076 | 0.0762 | 2.75E−05 |
| 1KG | rs1851808 | 2:76232489 | A | G | 0.6391 | 0.5946 | 0.601 | 0.5908 | 2.75E−05 |
| 1KG | rs13253938 | 8:136110326 | A | G | 0.4529 | 0.5041 | 0.5015 | 0.5043 | 2.96E−05 |
| 1KG | rs529478 | 18:53934507 | A | T | 0.4692 | 0.5086 | 0.5017 | 0.5109 | 3.33E−05 |
| 1KG | rs12798294 | 11:103362906 | C | G | 0.3164 | 0.2803 | 0.2821 | 0.2803 | 3.46E−05 |
| 1KG | chr8: 123521912 | 8:123521912 | A | G | 0.0874 | 0.1017 | 0.0967 | 0.1036 | 3.59E−05 |
| 1KG | rs28621471 | 4:86877248 | A | G | 0.3046 | 0.2786 | 0.2856 | 0.2751 | 3.73E−05 |
| 1KG | chr15: 47957127 | 15:47957127 | T | G | 0.097 | 0.0757 | 0.0702 | 0.0778 | 3.80E−05 |
| 1KG | chr5: 26212150 | 5:26212150 | T | C | 0.9194 | 0.9394 | 0.9405 | 0.9384 | 3.92E−05 |
| 1KG | chr2: 180796214 | 2:180796214 | A | C | 0.2095 | 0.1777 | 0.1783 | 0.177 | 3.93E−05 |
| 1KG | rs4967980 | 16:23091095 | T | C | 0.7845 | 0.7499 | 0.7452 | 0.7526 | 3.93E−05 |
| 1KG | chr6: 119117772 | 6:119117772 | T | C | 0.1086 | 0.0892 | 0.0797 | 0.0932 | 4.04E−05 |
| 1KG | rs12520598 | 5:74385314 | T | C | 0.1107 | 0.092 | 0.0856 | 0.0953 | 4.22E−05 |
| 1KG | chr10: 56380194 | 10:56380194 | A | G | 0.898 | 0.8876 | 0.8849 | 0.8888 | 4.33E−05 |
| 1KG | chr12: 8919954 | 12:8919954 | A | T | 0.9248 | 0.9348 | 0.9326 | 0.9358 | 4.66E−05 |
| 1KG | rs11592003 | 10:3945290 | T | C | 0.8209 | 0.85 | 0.8582 | 0.846 | 4.79E−05 |
| 1KG | rs235195 | 20:56979160 | A | G | 0.1339 | 0.1479 | 0.1444 | 0.15 | 5.07E−05 |
| 1KG | rs7623235 | 3:99135842 | A | T | 0.201 | 0.2268 | 0.2143 | 0.2319 | 5.19E−05 |
| 1KG | chr4: 23188024 | 4:23188024 | A | T | 0.1683 | 0.1431 | 0.1447 | 0.141 | 5.26E−05 |
| 1KG | rs12908430 | 15:40375218 | A | G | 0.162 | 0.1442 | 0.1401 | 0.1456 | 5.29E−05 |
| 1KG | chr17: 72483712 | 17:72483712 | A | G | 0.9281 | 0.939 | 0.9384 | 0.9389 | 5.30E−05 |
| 1KG | rs6679773 | 1:214700097 | T | C | 0.0688 | 0.0766 | 0.0787 | 0.0756 | 5.30E−05 |
| 1KG | chr1: 163986967 | 1:163986967 | T | C | 0.066 | 0.0553 | 0.0503 | 0.0575 | 5.42E−05 |
| 1KG | rs7958529 | 12:50881293 | C | G | 0.7298 | 0.7572 | 0.7584 | 0.7575 | 5.45E−05 |
| 1KG | chr2: 44132778 | 2:44132778 | A | G | 0.865 | 0.8855 | 0.8887 | 0.8844 | 5.50E−05 |
| 1KG | chr5: 113645446 | 5:113645446 | C | G | 0.8718 | 0.8494 | 0.8425 | 0.8529 | 5.54E−05 |
| 1KG | rs11067403 | 12:114010367 | T | C | 0.6488 | 0.6846 | 0.6629 | 0.6935 | 5.65E−05 |
| 1KG | rs343718 | 7:35595154 | A | C | 0.4303 | 0.3598 | 0.3814 | 0.3503 | 5.71E−05 |
| 1KG | rs6685751 | 1:227122574 | A | C | 0.4817 | 0.5145 | 0.5116 | 0.5159 | 6.00E−05 |
| 1KG | rs1492116 | 12:19878442 | T | C | 0.7597 | 0.7886 | 0.7765 | 0.7942 | 6.45E−05 |
| 1KG | rs5015852 | 6:132002096 | A | T | 0.8648 | 0.8791 | 0.8809 | 0.8781 | 6.50E−05 |
| 1KG | chr19: 14775139 | 19:14775139 | A | G | 0.1123 | 0.0947 | 0.0935 | 0.0949 | 6.58E−05 |
| 1KG | chr11: 124212812 | 11:124212812 | T | C | 0.0946 | 0.106 | 0.1074 | 0.1056 | 6.67E−05 |
| 1KG | rs10503493 | 8:14247492 | A | C | 0.0583 | 0.0416 | 0.0377 | 0.0439 | 6.78E−05 |
| 1KG | chr4: 86206508 | 4:86206508 | T | C | 0.0806 | 0.0669 | 0.0578 | 0.071 | 6.94E−05 |
| 1KG | rs2883171 | 5:21309600 | T | G | 0.8852 | 0.8609 | 0.8625 | 0.8605 | 7.25E−05 |
| 1KG | rs1360751 | 6:7152544 | A | C | 0.5214 | 0.5563 | 0.5644 | 0.5526 | 7.38E−05 |
| 1KG | rs2024393 | 16:11814579 | T | C | 0.1323 | 0.1506 | 0.1556 | 0.1482 | 7.45E−05 |
| 1KG | rs4260755 | 6:55456674 | A | C | 0.3446 | 0.3881 | 0.3802 | 0.3905 | 7.49E−05 |
| 1KG | rs17408651 | 1:79360392 | T | G | 0.1211 | 0.1057 | 0.1072 | 0.1044 | 7.50E−05 |
| 1KG | rs13192030 | 6:109470664 | A | T | 0.8935 | 0.9066 | 0.9076 | 0.9058 | 7.84E−05 |
| 1KG | rs12632105 | 3:130591125 | C | G | 0.165 | 0.1818 | 0.2174 | 0.1663 | 8.06E−05 |
| 1KG | chr1: 85757359 | 1:85757359 | T | C | 0.1217 | 0.1063 | 0.1025 | 0.1082 | 8.26E−05 |
| 1KG | chr10: 57479107 | 10:57479107 | A | C | 0.0961 | 0.1202 | 0.1232 | 0.1194 | 8.31E−05 |
| 1KG | rs7094579 | 10:92382787 | T | C | 0.064 | 0.045 | 0.0467 | 0.0446 | 8.38E−05 |
| 1KG | chr15: 35201758 | 15:35201758 | T | G | 0.1305 | 0.1462 | 0.1421 | 0.1479 | 8.75E−05 |
| 1KG | chr6: 162467903 | 6:162467903 | A | G | 0.1204 | 0.0945 | 0.1012 | 0.092 | 8.90E−05 |
| 1KG | chr8: 40099493 | 8:40099493 | A | T | 0.9144 | 0.9013 | 0.9023 | 0.9012 | 8.92E−05 |
| 1KG | rs16822447 | 3:154434339 | A | T | 0.8267 | 0.8484 | 0.8354 | 0.8546 | 9.03E−05 |
| 1KG | rs135912 | 22:43172593 | A | G | 0.828 | 0.8533 | 0.851 | 0.8544 | 9.13E−05 |
| 1KG | chr5: 149749881 | 5:149749881 | C | G | 0.0572 | 0.0765 | 0.0778 | 0.0755 | 9.18E−05 |
| 1KG | rs2135548 | 3:71584585 | A | G | 0.7682 | 0.7325 | 0.729 | 0.7345 | 9.36E−05 |
| 1KG | rs1358395 | 7:86076557 | A | G | 0.3321 | 0.3001 | 0.2996 | 0.3008 | 9.41E−05 |
| 1KG | chr3: 126793536 | 3:126793536 | A | C | 0.9216 | 0.9328 | 0.934 | 0.9324 | 9.64E−05 |
| 1KG | rs625761 | 11:100149779 | A | G | 0.7544 | 0.7843 | 0.8014 | 0.7773 | 9.68E−05 |
| 1KG | rs1540819 | 3:118210505 | T | C | 0.4972 | 0.5379 | 0.518 | 0.546 | 9.74E−05 |
| 1KG | rs4073997 | 17:77136846 | C | G | 0.2583 | 0.2845 | 0.2673 | 0.2923 | 9.79E−05 |
| 1KG | chr1: 59476907 | 1:59476907 | A | G | 0.0974 | 0.0879 | 0.0854 | 0.0891 | 9.88E−05 |
| HM3 | rs16965939 | 15:35992085 | T | C | 0.1076 | 0.085 | 0.0803 | 0.0862 | 1.11E−06 |
| HM3 | rs1999930 | 6:116493827 | T | C | 0.3083 | 0.261 | 0.2599 | 0.261 | 2.22E−06 |
| HM3 | rs1789110 | 18:72988032 | A | C | 0.6301 | 0.5853 | 0.5554 | 0.5993 | 2.35E−06 |
| HM3 | rs10280782 | 7:23550807 | A | G | 0.8464 | 0.8778 | 0.8639 | 0.8833 | 2.61E−06 |
| HM3 | rs1079982 | 22:36259705 | T | G | 0.0699 | 0.046 | 0.0433 | 0.0473 | 3.01E−06 |
| HM3 | rs11854658 | 15:87312556 | A | G | 0.636 | 0.6783 | 0.6726 | 0.6794 | 3.87E−06 |
| HM3 | rs8076470 | 17:45980827 | T | C | 0.3673 | 0.404 | 0.4037 | 0.4043 | 3.98E−06 |
| HM3 | rs8053796 | 16:74921678 | T | C | 0.2393 | 0.2701 | 0.2808 | 0.2653 | 4.39E−06 |
| HM3 | rs2037156 | 6:93573222 | A | G | 0.8273 | 0.7909 | 0.7932 | 0.7901 | 7.94E−06 |
| HM3 | rs3760775 | 19:5792356 | T | G | 0.0675 | 0.0485 | 0.0463 | 0.0498 | 1.27E−05 |
| HM3 | rs9291737 | 5:61112540 | A | T | 0.5787 | 0.6184 | 0.6177 | 0.6213 | 1.30E−05 |
| HM3 | rs13094238 | 3:156261403 | T | C | 0.8449 | 0.8656 | 0.8752 | 0.8612 | 1.36E−05 |
| HM3 | rs2730613 | 7:43880667 | A | C | 0.6269 | 0.6643 | 0.6733 | 0.6613 | 1.47E−05 |
| HM3 | rs10103808 | 8:139212254 | A | T | 0.9102 | 0.8883 | 0.8914 | 0.8876 | 1.92E−05 |
| HM3 | rs7720497 | 5:5291813 | C | G | 0.9484 | 0.9279 | 0.9277 | 0.9281 | 2.05E−05 |
| HM3 | rs6531212 | 2:20201501 | T | C | 0.3554 | 0.3169 | 0.3261 | 0.3121 | 2.13E−05 |
| HM3 | rs8011890 | 14:95216646 | A | C | 0.0797 | 0.1028 | 0.1059 | 0.1007 | 2.34E−05 |
| HM3 | rs12926103 | 16:84929276 | A | G | 0.0569 | 0.0764 | 0.0767 | 0.0775 | 2.67E−05 |
| HM3 | rs16971055 | 17:73838135 | A | G | 0.0849 | 0.0976 | 0.1005 | 0.0965 | 2.83E−05 |
| HM3 | rs1912795 | 13:30736688 | A | G | 0.4275 | 0.4639 | 0.4618 | 0.4659 | 3.21E−05 |
| HM3 | rs2127740 | 16:72289810 | A | G | 0.1145 | 0.1393 | 0.1393 | 0.1383 | 3.30E−05 |
| HM3 | rs17155281 | 7:27475356 | T | C | 0.69 | 0.719 | 0.7299 | 0.7141 | 3.34E−05 |
| HM3 | rs4256145 | 3:163192835 | A | G | 0.6505 | 0.694 | 0.702 | 0.6903 | 3.62E−05 |
| HM3 | rs6587759 | 1:247130153 | A | G | 0.0579 | 0.0711 | 0.066 | 0.0736 | 4.17E−05 |
| HM3 | rs2052572 | 19:36639376 | A | G | 0.3041 | 0.3451 | 0.3493 | 0.3435 | 4.43E−05 |
| HM3 | rs13278062 | 8:23138916 | T | G | 0.5113 | 0.5457 | 0.5218 | 0.5569 | 5.01E−05 |
| HM3 | rs722782 | 8:506479 | A | C | 0.1081 | 0.0836 | 0.065 | 0.0915 | 5.22E−05 |
| HM3 | rs1867348 | 6:160376153 | T | C | 0.0723 | 0.0979 | 0.1091 | 0.0934 | 5.23E−05 |
| HM3 | rs9461856 | 6:33503177 | A | G | 0.5248 | 0.4881 | 0.478 | 0.4934 | 5.32E−05 |
| HM3 | rs2270637 | 8:20081107 | C | G | 0.8007 | 0.8279 | 0.8311 | 0.8271 | 5.44E−05 |
| HM3 | rs2798832 | 10:8226099 | T | C | 0.2312 | 0.2625 | 0.2636 | 0.2613 | 5.54E−05 |
| HM3 | rs12632671 | 3:53233464 | A | G | 0.0413 | 0.0583 | 0.044 | 0.0636 | 5.73E−05 |
| HM3 | rs9713311 | 3:120558458 | T | C | 0.6302 | 0.5938 | 0.5841 | 0.5983 | 5.87E−05 |
| HM3 | rs7626245 | 3:101053451 | C | G | 0.0642 | 0.0869 | 0.0884 | 0.0869 | 6.63E−05 |
| HM3 | rs513683 | 11:73662041 | C | G | 0.4594 | 0.4218 | 0.4183 | 0.4237 | 7.56E−05 |
| HM3 | rs2376241 | 4:36840346 | T | G | 0.2416 | 0.2741 | 0.2854 | 0.2706 | 7.95E−05 |
| HM3 | rs251525 | 5:52621268 | A | G | 0.6907 | 0.6564 | 0.6454 | 0.6633 | 8.06E−05 |
| HM3 | rs1737478 | 1:165795961 | T | C | 0.2189 | 0.2486 | 0.2452 | 0.2501 | 8.47E−05 |
| HM3 | rs7783337 | 7:14123511 | A | G | 0.8694 | 0.8452 | 0.8345 | 0.8501 | 8.84E−05 |
| HM3 | rs10225927 | 7:54736904 | A | G | 0.8098 | 0.7807 | 0.7796 | 0.7809 | 9.06E−05 |
| HM3 | rs164700 | 5:106936632 | A | C | 0.1396 | 0.1709 | 0.1727 | 0.1696 | 9.26E−05 |
| HM3 | rs10848645 | 12:2290505 | A | G | 0.5848 | 0.5515 | 0.5417 | 0.5561 | 9.39E−05 |

| IMP | SNP | P_1vs5 | P_1vs4 | P_4vs5 | OR_1vs45 | OR_1vs5 | OR_1vs4 |
|---|---|---|---|---|---|---|---|
| 1KG | rs1367068 | 2.18E−06 | 0.001938 | 0.6018 | 1.3457 | 1.3741 | 1.3071 |
| 1KG | rs1443179 | 0.0004505 | 1.73E−06 | 0.1242 | 1.6593 | 1.5105 | 1.9613 |
| 1KG | rs9328048 | 1.12E−05 | 0.002746 | 0.7665 | 0.7763 | 0.7699 | 0.7915 |
| 1KG | chr4: 182189551 | 3.87E−07 | 0.04747 | 0.03589 | 0.5758 | 0.5205 | 0.7122 |
| 1KG | rs1915098 | 0.0004493 | 5.65E−05 | 0.1709 | 0.7849 | 0.8175 | 0.7324 |
| 1KG | rs1418473 | 0.002335 | 2.07E−07 | 0.02957 | 1.5676 | 1.3911 | 1.9294 |
| 1KG | rs11183802 | 6.17E−07 | 0.05563 | 0.116 | 1.4459 | 1.5481 | 1.2508 |
| 1KG | rs728371 | 8.69E−05 | 0.0003392 | 0.4542 | 1.2293 | 1.2178 | 1.2604 |
| 1KG | chr11: 1768072 | 2.80E−05 | 0.005699 | 0.6279 | 0.7511 | 0.7449 | 0.7758 |
| 1KG | rs11596472 | 0.0007817 | 4.20E−06 | 0.03616 | 0.7061 | 0.7516 | 0.6049 |
| 1KG | rs6982567 | 4.59E−06 | 0.008215 | 0.4558 | 1.3214 | 1.3598 | 1.2698 |
| 1KG | rs2277252 | 4.35E−05 | 0.001441 | 0.9691 | 1.2345 | 1.2354 | 1.239 |
| 1KG | rs8091635 | 1.81E−07 | 0.2065 | 0.009328 | 1.2464 | 1.3295 | 1.0944 |
| 1KG | rs9366769 | 0.0002831 | 0.0004622 | 0.3999 | 1.2501 | 1.2274 | 1.2983 |
| 1KG | rs5771717 | 0.08595 | 3.48E−11 | 7.10E−07 | 1.3632 | 1.1502 | 1.8242 |
| 1KG | chr22: 33891064 | 3.12E−05 | 0.0119 | 0.4455 | 0.5118 | 0.4814 | 0.5707 |
| 1KG | chr5: 165133520 | 4.59E−05 | 0.004485 | 0.6401 | 1.2744 | 1.2886 | 1.257 |
| 1KG | chr3: 116361297 | 1.99E−05 | 0.0154 | 0.5013 | 0.7342 | 0.7127 | 0.7793 |
| 1KG | rs17071512 | 5.38E−06 | 0.04102 | 0.22 | 1.4055 | 1.4971 | 1.2542 |
| 1KG | rs1384044 | 0.0003344 | 0.001241 | 0.7053 | 0.8377 | 0.8481 | 0.8247 |
| 1KG | rs9308649 | 0.0008531 | 0.00026 | 0.3382 | 1.2416 | 1.2059 | 1.3153 |
| 1KG | rs10067691 | 2.20E−05 | 0.01173 | 0.49 | 0.6712 | 0.6389 | 0.7115 |
| 1KG | chr1: 100166675 | 0.0002956 | 0.0004285 | 0.4504 | 1.4999 | 1.4689 | 1.5723 |
| 1KG | rs10768315 | 0.0004323 | 0.003677 | 0.9683 | 0.8058 | 0.8205 | 0.8053 |
| 1KG | chr10: 88344492 | 2.88E−05 | 0.01875 | 0.3949 | 1.2872 | 1.3154 | 1.2204 |
| 1KG | rs7260457 | 0.00104 | 0.0002468 | 0.2306 | 0.6139 | 0.6586 | 0.5268 |
| 1KG | chr12: 11233234 | 0.0001776 | 0.002787 | 0.7035 | 1.2854 | 1.2752 | 1.2883 |
| 1KG | rs2491146 | 0.001371 | 0.0002038 | 0.2376 | 0.8228 | 0.8478 | 0.7798 |
| 1KG | chr16: 17310751 | 0.0002343 | 0.004891 | 0.8523 | 1.392 | 1.3852 | 1.3891 |
| 1KG | rs4711751 | 0.0007193 | 0.0006112 | 0.6538 | 1.2192 | 1.1931 | 1.2684 |
| 1KG | chr11: 104185708 | 0.0001158 | 0.007171 | 0.7311 | 2.1902 | 2.2292 | 2.054 |
| 1KG | rs1851808 | 6.83E−05 | 0.01279 | 0.4495 | 0.8368 | 0.8272 | 0.8593 |
| 1KG | rs13253938 | 0.0002487 | 0.003161 | 0.8557 | 1.2071 | 1.2036 | 1.2116 |
| 1KG | rs529478 | 0.0001374 | 0.01656 | 0.4809 | 1.2104 | 1.2154 | 1.1726 |
| 1KG | rs12798294 | 0.0002762 | 0.00827 | 0.8248 | 0.825 | 0.8273 | 0.8369 |
| 1KG | chr8: 123521912 | 4.13E−05 | 0.04697 | 0.1884 | 1.7769 | 1.8751 | 1.4959 |
| 1KG | rs28621471 | 1.86E−05 | 0.0351 | 0.3624 | 0.7613 | 0.7297 | 0.8166 |
| 1KG | chr15: 47957127 | 0.001928 | 0.0001134 | 0.1374 | 0.6679 | 0.7123 | 0.5535 |
| 1KG | chr5: 26212150 | 0.0001975 | 0.0042 | 0.8515 | 1.4249 | 1.4366 | 1.4542 |
| 1KG | chr2: 180796214 | 0.0001339 | 0.006444 | 0.9313 | 0.808 | 0.7997 | 0.8129 |
| 1KG | rs4967980 | 0.0005012 | 0.001415 | 0.5372 | 0.815 | 0.825 | 0.7971 |
| 1KG | chr6: 119117772 | 0.005895 | 4.55E−05 | 0.0698 | 0.7144 | 0.7789 | 0.5954 |
| 1KG | rs12520598 | 0.001511 | 0.0006204 | 0.1961 | 0.7033 | 0.7367 | 0.637 |
| 1KG | chr10: 56380194 | 0.0007999 | 0.001293 | 0.6541 | 0.5967 | 0.6266 | 0.5643 |
| 1KG | chr12: 8919954 | 7.18E−05 | 0.03968 | 0.3457 | 2.0839 | 2.2434 | 1.7108 |
| 1KG | rs11592003 | 0.001862 | 0.0004429 | 0.2299 | 1.2621 | 1.2195 | 1.3556 |
| 1KG | rs235195 | 1.40E−05 | 0.05916 | 0.2021 | 1.369 | 1.4506 | 1.2351 |
| 1KG | rs7623235 | 2.27E−05 | 0.08935 | 0.127 | 1.2556 | 1.301 | 1.149 |
| 1KG | chr4: 23188024 | 0.0001418 | 0.008275 | 0.6592 | 0.776 | 0.7626 | 0.7846 |
| 1KG | rs12908430 | 0.001121 | 0.001758 | 0.6615 | 0.7128 | 0.7338 | 0.6801 |
| 1KG | chr17: 72483712 | 0.0001566 | 0.02176 | 0.6626 | 1.5837 | 1.6182 | 1.474 |
| 1KG | rs6679773 | 0.0007085 | NA | 0.2521 | 2.7548 | 2.4969 | NA |
| 1KG | chr1: 163986967 | 0.002678 | 0.0001905 | 0.1079 | 0.5731 | 0.6343 | 0.4391 |
| 1KG | rs7958529 | 0.0002342 | 0.005289 | 0.9156 | 1.2499 | 1.2553 | 1.2528 |
| 1KG | chr2: 44132778 | 0.0007818 | 0.00185 | 0.6138 | 1.4121 | 1.38 | 1.4963 |
| 1KG | chr5: 113645446 | 0.001329 | 0.0003113 | 0.3121 | 0.7765 | 0.7985 | 0.7276 |
| 1KG | rs11067403 | 6.74E−06 | 0.3045 | 0.02498 | 1.1924 | 1.2475 | 1.0668 |
| 1KG | rs343718 | 9.88E−05 | 0.03219 | 0.4837 | 0.8397 | 0.8269 | 0.8738 |
| 1KG | rs6685751 | 0.0001216 | 0.007157 | 0.9407 | 1.266 | 1.285 | 1.2611 |
| 1KG | rs1492116 | 2.06E−05 | 0.07507 | 0.1982 | 1.2137 | 1.2623 | 1.1328 |
| 1KG | rs5015852 | 0.0009123 | 0.001753 | 0.4059 | 1.716 | 1.644 | 1.8914 |
| 1KG | chr19: 14775139 | 0.0004184 | 0.004882 | 0.8877 | 0.708 | 0.7105 | 0.6955 |
| 1KG | chr11: 124212812 | 0.0005409 | 0.003229 | 0.8919 | 1.5975 | 1.5668 | 1.6409 |
| 1KG | rs10503493 | 0.001999 | 0.001334 | 0.2556 | 0.6846 | 0.7204 | 0.6176 |
| 1KG | chr4: 86206508 | 0.01383 | 8.39E−06 | 0.006801 | 0.6116 | 0.7161 | 0.4061 |
| 1KG | rs2883171 | 0.0002656 | 0.008738 | 0.7083 | 0.778 | 0.7754 | 0.7889 |
| 1KG | rs1360751 | 0.001726 | 0.000897 | 0.4273 | 1.1766 | 1.1543 | 1.2186 |
| 1KG | rs2024393 | 0.00273 | 4.13E−05 | 0.05154 | 1.5306 | 1.4228 | 1.8503 |
| 1KG | rs4260755 | 0.0002045 | 0.01893 | 0.6182 | 1.2012 | 1.2095 | 1.1714 |
| 1KG | rs17408651 | 0.0001251 | 0.01571 | 0.6615 | 0.6938 | 0.6705 | 0.72 |
| 1KG | rs13192030 | 0.001041 | 0.002687 | 0.5341 | 1.6219 | 1.565 | 1.7468 |
| 1KG | rs12632105 | 0.1604 | 2.08E−08 | 8.73E−05 | 1.2491 | 1.0953 | 1.5345 |
| 1KG | chr1: 85757359 | 0.00292 | 0.0008545 | 0.2963 | 0.6962 | 0.7345 | 0.6301 |
| 1KG | chr10: 57479107 | 0.0008563 | 0.001329 | 0.7289 | 1.2908 | 1.272 | 1.3383 |
| 1KG | rs7094579 | 0.0003193 | 0.02934 | 0.6434 | 0.6999 | 0.6898 | 0.7454 |
| 1KG | chr15: 35201758 | 4.56E−05 | 0.06212 | 0.3605 | 1.3907 | 1.4609 | 1.2538 |
| 1KG | chr6: 162467903 | 9.40E−05 | 0.1299 | 0.1733 | 0.7609 | 0.7339 | 0.8582 |
| 1KG | chr8: 40099493 | 0.0003539 | 0.00656 | 0.9092 | 0.614 | 0.6125 | 0.6257 |
| 1KG | rs16822447 | 1.06E−05 | 0.1821 | 0.05002 | 1.2811 | 1.3679 | 1.1293 |
| 1KG | rs135912 | 0.0003393 | 0.01279 | 0.7976 | 1.2967 | 1.306 | 1.2762 |
| 1KG | chr5: 149749881 | 0.0003726 | 0.004751 | 0.8373 | 1.3802 | 1.3859 | 1.3849 |
| 1KG | rs2135548 | 0.0007483 | 0.003939 | 0.6911 | 0.8336 | 0.8399 | 0.8252 |
| 1KG | rs1358395 | 0.0002692 | 0.007593 | 0.9665 | 0.8423 | 0.8352 | 0.8421 |
| 1KG | chr3: 126793536 | 0.002748 | 0.001379 | 0.5783 | 1.8746 | 1.7224 | 2.1493 |
| 1KG | rs625761 | 0.008243 | 4.33E−05 | 0.04509 | 1.2134 | 1.1575 | 1.3536 |
| 1KG | rs1540819 | 3.59E−05 | 0.1277 | 0.0718 | 1.172 | 1.2073 | 1.0942 |
| 1KG | rs4073997 | 9.93E−07 | 0.4996 | 0.01417 | 1.2642 | 1.3908 | 1.0609 |
| 1KG | chr1: 59476907 | 0.001353 | 0.001893 | 0.5084 | 0.571 | 0.5938 | 0.5152 |
| HM3 | rs16965939 | 6.28E−05 | 9.95E−05 | 0.4187 | 0.6414 | 0.6589 | 0.5837 |
| HM3 | rs1999930 | 1.79E−05 | 0.001342 | 0.9695 | 0.8066 | 0.804 | 0.8089 |
| HM3 | rs1789110 | 0.003144 | 8.23E−08 | 0.002278 | 0.8206 | 0.8707 | 0.7249 |
| HM3 | rs10280782 | 7.34E−07 | 0.0713 | 0.04504 | 1.3258 | 1.4061 | 1.1675 |
| HM3 | rs1079982 | 0.0001407 | 0.0006923 | 0.4598 | 0.6454 | 0.6686 | 0.6092 |
| HM3 | rs11854658 | 2.53E−05 | 0.005429 | 0.6806 | 1.2181 | 1.2244 | 1.1896 |
| HM3 | rs8076470 | 2.31E−05 | 0.001837 | 0.8181 | 1.2139 | 1.2196 | 1.2087 |
| HM3 | rs8053796 | 0.0002953 | 4.88E−05 | 0.1967 | 1.2672 | 1.2299 | 1.3492 |
| HM3 | rs2037156 | 4.59E−05 | 0.004051 | 0.6462 | 0.7899 | 0.7875 | 0.8049 |
| HM3 | rs3760775 | 0.0002587 | 0.001212 | 0.4955 | 0.6626 | 0.6787 | 0.6266 |
| HM3 | rs9291737 | 7.45E−05 | 0.002397 | 0.6956 | 1.1971 | 1.2005 | 1.2005 |
| HM3 | rs13094238 | 0.003017 | 6.57E−06 | 0.02605 | 1.4804 | 1.345 | 1.8713 |
| HM3 | rs2730613 | 0.0003072 | 0.0003156 | 0.3924 | 1.2048 | 1.1886 | 1.2562 |
| HM3 | rs10103808 | 4.36E−05 | 0.007698 | 0.8279 | 0.7472 | 0.7342 | 0.7717 |
| HM3 | rs7720497 | 9.58E−05 | 0.002133 | 0.956 | 0.6878 | 0.6842 | 0.6888 |
| HM3 | rs6531212 | 1.54E−05 | 0.04856 | 0.2214 | 0.8334 | 0.8124 | 0.8837 |
| HM3 | rs8011890 | 0.0004883 | 0.0002716 | 0.3724 | 1.4104 | 1.3647 | 1.5103 |
| HM3 | rs12926103 | 0.0001097 | 0.003454 | 0.8956 | 1.4075 | 1.415 | 1.3989 |
| HM3 | rs16971055 | 0.0005136 | 0.0009973 | 0.6898 | 1.5452 | 1.494 | 1.6131 |
| HM3 | rs1912795 | 6.87E−05 | 0.01009 | 0.8198 | 1.1918 | 1.2068 | 1.1709 |
| HM3 | rs2127740 | 0.0002762 | 0.002813 | 0.9732 | 1.2942 | 1.2848 | 1.3023 |
| HM3 | rs17155281 | 0.001612 | 0.0001765 | 0.1384 | 1.2603 | 1.2171 | 1.3623 |
| HM3 | rs4256145 | 0.0004254 | 0.001256 | 0.5498 | 1.1948 | 1.1844 | 1.2273 |
| HM3 | rs6587759 | 2.45E−05 | 0.05012 | 0.2593 | 1.4444 | 1.5104 | 1.2886 |
| HM3 | rs2052572 | 0.0002378 | 0.003662 | 0.8437 | 1.1942 | 1.195 | 1.1989 |
| HM3 | rs13278062 | 1.40E−06 | 0.345 | 0.01412 | 1.2206 | 1.3017 | 1.0697 |
| HM3 | rs722782 | 0.01492 | 1.21E−06 | 0.001233 | 0.7568 | 0.8319 | 0.5781 |
| HM3 | rs1867348 | 0.003738 | 1.45E−05 | 0.04783 | 1.3447 | 1.2675 | 1.5481 |
| HM3 | rs9461856 | 0.001189 | 0.0004144 | 0.3166 | 0.849 | 0.863 | 0.8126 |
| HM3 | rs2270637 | 0.0004731 | 0.002846 | 0.7084 | 1.2348 | 1.2282 | 1.2596 |
| HM3 | rs2798832 | 0.0001696 | 0.006426 | 0.9931 | 1.2184 | 1.2283 | 1.2099 |
| HM3 | rs12632671 | 2.52E−06 | 0.5699 | 0.002276 | 1.5035 | 1.6643 | 1.0939 |
| HM3 | rs9713311 | 0.001113 | 0.0003867 | 0.4046 | 0.8472 | 0.8606 | 0.8096 |
| HM3 | rs7626245 | 0.0001847 | 0.005226 | 0.8963 | 1.3626 | 1.377 | 1.3483 |
| HM3 | rs513683 | 0.0008254 | 0.003938 | 0.7375 | 0.8496 | 0.8578 | 0.8406 |
| HM3 | rs2376241 | 0.0009555 | 0.0004383 | 0.2413 | 1.201 | 1.1867 | 1.2611 |
| HM3 | rs251525 | 0.004244 | 0.0004694 | 0.3072 | 0.842 | 0.8698 | 0.8035 |
| HM3 | rs1737478 | 0.00017 | 0.02088 | 0.6004 | 1.2064 | 1.2208 | 1.1724 |
| HM3 | rs7783337 | 0.003599 | 0.0001757 | 0.1258 | 0.7876 | 0.8221 | 0.7259 |
| HM3 | rs10225927 | 0.0003114 | 0.004791 | 0.9041 | 0.8221 | 0.8187 | 0.8162 |
| HM3 | rs164700 | 0.001207 | 0.001887 | 0.6632 | 1.268 | 1.242 | 1.3067 |
| HM3 | rs10848645 | 0.003688 | 0.0005319 | 0.3002 | 0.8371 | 0.863 | 0.7959 |

| IMP | SNP | OR_4vs5 | SE_1vs45 | SE_1vs5 | SE_1vs4 | SE_4vs5 | INFO_1vs45 |
|---|---|---|---|---|---|---|---|
| 1KG | rs1367068 | 1.05 | 0.0593 | 0.0671 | 0.0864 | 0.0936 | 0.7032 |
| 1KG | rs1443179 | 0.8045 | 0.1051 | 0.1176 | 0.1408 | 0.1415 | 0.5996 |
| 1KG | rs9328048 | 0.9758 | 0.0535 | 0.0595 | 0.0781 | 0.0824 | 0.603 |
| 1KG | chr4: 182189551 | 0.6905 | 0.1174 | 0.1287 | 0.1713 | 0.1765 | 0.3073 |
| 1KG | rs1915098 | 1.1208 | 0.0517 | 0.0574 | 0.0773 | 0.0833 | 0.9527 |
| 1KG | rs1418473 | 0.7574 | 0.0963 | 0.1085 | 0.1266 | 0.1277 | 0.7571 |
| 1KG | rs11183802 | 1.209 | 0.0801 | 0.0877 | 0.1169 | 0.1208 | 0.6692 |
| 1KG | rs728371 | 0.9512 | 0.0451 | 0.0502 | 0.0646 | 0.0669 | 0.912 |
| 1KG | chr11: 1768072 | 0.9527 | 0.0626 | 0.0703 | 0.0918 | 0.1 | 0.7808 |
| 1KG | rs11596472 | 1.2672 | 0.0769 | 0.085 | 0.1093 | 0.113 | 0.4398 |
| 1KG | rs6982567 | 1.0684 | 0.0616 | 0.0671 | 0.0904 | 0.0887 | 0.8663 |
| 1KG | rs2277252 | 1.0028 | 0.0467 | 0.0517 | 0.0673 | 0.0714 | 0.7486 |
| 1KG | rs8091635 | 1.2164 | 0.0493 | 0.0546 | 0.0714 | 0.0753 | 0.7131 |
| 1KG | rs9366769 | 0.935 | 0.0504 | 0.0564 | 0.0746 | 0.0798 | 0.9597 |
| 1KG | rs5771717 | 0.619 | 0.07 | 0.0815 | 0.0907 | 0.0967 | 0.9131 |
| 1KG | chr22: 33891064 | 0.8256 | 0.1516 | 0.1755 | 0.223 | 0.2512 | 0.2964 |
| 1KG | chr5: 165133520 | 1.0415 | 0.0549 | 0.0622 | 0.0805 | 0.0869 | 0.939 |
| 1KG | chr3: 116361297 | 0.9288 | 0.0702 | 0.0794 | 0.1029 | 0.1098 | 0.5552 |
| 1KG | rs17071512 | 1.1656 | 0.0773 | 0.0887 | 0.1108 | 0.125 | 0.7041 |
| 1KG | rs1384044 | 1.0241 | 0.0411 | 0.0459 | 0.0597 | 0.0629 | 0.9648 |
| 1KG | rs9308649 | 0.9256 | 0.0503 | 0.0561 | 0.075 | 0.0807 | 1.0129 |
| 1KG | rs10067691 | 0.9027 | 0.0927 | 0.1056 | 0.1351 | 0.1483 | 0.5304 |
| 1KG | chr1: 100166675 | 0.9069 | 0.0946 | 0.1062 | 0.1285 | 0.1294 | 0.9209 |
| 1KG | rs10768315 | 0.9969 | 0.0506 | 0.0562 | 0.0746 | 0.0789 | 0.9621 |
| 1KG | chr10: 88344492 | 1.0785 | 0.0592 | 0.0655 | 0.0847 | 0.0888 | 0.91 |
| 1KG | rs7260457 | 1.2508 | 0.1146 | 0.1273 | 0.1748 | 0.1867 | 0.1927 |
| 1KG | chr12: 11233234 | 0.9674 | 0.0591 | 0.0648 | 0.0847 | 0.087 | 0.5826 |
| 1KG | rs2491146 | 1.0885 | 0.0461 | 0.0516 | 0.067 | 0.0718 | 0.7785 |
| 1KG | chr16: 17310751 | 0.9766 | 0.0787 | 0.0886 | 0.1168 | 0.1273 | 0.7538 |
| 1KG | rs4711751 | 0.9686 | 0.0472 | 0.0522 | 0.0694 | 0.0711 | 0.7326 |
| 1KG | chr11: 104185708 | 1.0996 | 0.187 | 0.208 | 0.2677 | 0.2762 | 0.1749 |
| 1KG | rs1851808 | 0.9521 | 0.0425 | 0.0476 | 0.0609 | 0.0649 | 0.9525 |
| 1KG | rs13253938 | 0.9875 | 0.0451 | 0.0506 | 0.065 | 0.0693 | 0.8145 |
| 1KG | rs529478 | 1.0503 | 0.046 | 0.0512 | 0.0665 | 0.0697 | 0.7669 |
| 1KG | rs12798294 | 0.9841 | 0.0465 | 0.0521 | 0.0674 | 0.0726 | 0.9005 |
| 1KG | chr8: 123521912 | 1.3131 | 0.1391 | 0.1533 | 0.2027 | 0.2071 | 0.2493 |
| 1KG | rs28621471 | 0.9115 | 0.0661 | 0.0736 | 0.0962 | 0.1018 | 0.4485 |
| 1KG | chr15: 47957127 | 1.2761 | 0.098 | 0.1094 | 0.1532 | 0.1641 | 0.548 |
| 1KG | chr5: 26212150 | 0.9743 | 0.0861 | 0.0973 | 0.1308 | 0.1388 | 0.8648 |
| 1KG | chr2: 180796214 | 1.007 | 0.0519 | 0.0585 | 0.076 | 0.0812 | 0.9746 |
| 1KG | rs4967980 | 1.0468 | 0.0498 | 0.0553 | 0.071 | 0.0741 | 0.9209 |
| 1KG | chr6: 119117772 | 1.2821 | 0.0819 | 0.0908 | 0.1272 | 0.1371 | 0.6885 |
| 1KG | rs12520598 | 1.1972 | 0.0859 | 0.0963 | 0.1318 | 0.1392 | 0.6284 |
| 1KG | chr10: 56380194 | 1.0826 | 0.1263 | 0.1394 | 0.1778 | 0.1771 | 0.2768 |
| 1KG | chr12: 8919954 | 1.3077 | 0.1803 | 0.2035 | 0.261 | 0.2845 | 0.1932 |
| 1KG | rs11592003 | 0.8948 | 0.0573 | 0.0638 | 0.0866 | 0.0926 | 0.9071 |
| 1KG | rs235195 | 1.1619 | 0.0775 | 0.0856 | 0.1119 | 0.1176 | 0.5644 |
| 1KG | rs7623235 | 1.1385 | 0.0562 | 0.0621 | 0.0818 | 0.085 | 0.7698 |
| 1KG | chr4: 23188024 | 0.957 | 0.0627 | 0.0712 | 0.0919 | 0.0996 | 0.795 |
| 1KG | rs12908430 | 1.0615 | 0.0838 | 0.095 | 0.1233 | 0.1362 | 0.463 |
| 1KG | chr17: 72483712 | 1.0827 | 0.1137 | 0.1273 | 0.1691 | 0.1821 | 0.5091 |
| 1KG | rs6679773 | 0.7295 | 0.2507 | 0.2702 | NA | 0.2753 | 0.1192 |
| 1KG | chr1: 163986967 | 1.4572 | 0.1379 | 0.1516 | 0.2206 | 0.2342 | 0.3787 |
| 1KG | rs7958529 | 0.9908 | 0.0553 | 0.0618 | 0.0808 | 0.087 | 0.6987 |
| 1KG | chr2: 44132778 | 0.9334 | 0.0856 | 0.0959 | 0.1294 | 0.1366 | 0.5156 |
| 1KG | chr5: 113645446 | 1.0967 | 0.0627 | 0.0701 | 0.0882 | 0.0913 | 0.8665 |
| 1KG | rs11067403 | 1.163 | 0.0437 | 0.0491 | 0.063 | 0.0673 | 0.9642 |
| 1KG | rs343718 | 0.9543 | 0.0434 | 0.0488 | 0.063 | 0.0669 | 0.9767 |
| 1KG | rs6685751 | 1.0067 | 0.0588 | 0.0653 | 0.0863 | 0.0895 | 0.474 |
| 1KG | rs1492116 | 1.101 | 0.0485 | 0.0547 | 0.0701 | 0.0748 | 1.0041 |
| 1KG | rs5015852 | 0.8311 | 0.1352 | 0.1499 | 0.2037 | 0.2225 | 0.2025 |
| 1KG | chr19: 14775139 | 1.0195 | 0.0865 | 0.0969 | 0.129 | 0.1367 | 0.5933 |
| 1KG | chr11: 124212812 | 0.9775 | 0.1175 | 0.1298 | 0.1682 | 0.1678 | 0.3298 |
| 1KG | rs10503493 | 1.1983 | 0.0951 | 0.1061 | 0.1502 | 0.1591 | 0.9801 |
| 1KG | chr4: 86206508 | 1.7899 | 0.1236 | 0.1357 | 0.2023 | 0.2151 | 0.4013 |
| 1KG | rs2883171 | 0.9659 | 0.0633 | 0.0698 | 0.0904 | 0.0927 | 0.9269 |
| 1KG | rs1360751 | 0.9508 | 0.041 | 0.0458 | 0.0595 | 0.0636 | 0.975 |
| 1KG | rs2024393 | 0.745 | 0.1074 | 0.1177 | 0.1501 | 0.1512 | 0.3005 |
| 1KG | rs4260755 | 1.0355 | 0.0463 | 0.0512 | 0.0674 | 0.07 | 0.8224 |
| 1KG | rs17408651 | 0.9376 | 0.0923 | 0.1042 | 0.136 | 0.147 | 0.4815 |
| 1KG | rs13192030 | 0.8833 | 0.1225 | 0.1366 | 0.1858 | 0.1996 | 0.3052 |
| 1KG | rs12632105 | 0.7223 | 0.0564 | 0.0649 | 0.0764 | 0.0829 | 0.9082 |
| 1KG | chr1: 85757359 | 1.1698 | 0.092 | 0.1037 | 0.1385 | 0.1502 | 0.4938 |
| 1KG | chr10: 57479107 | 0.9682 | 0.0649 | 0.0722 | 0.0908 | 0.0933 | 1.0046 |
| 1KG | rs7094579 | 0.934 | 0.0907 | 0.1032 | 0.1348 | 0.1474 | 0.998 |
| 1KG | chr15: 35201758 | 1.1228 | 0.0841 | 0.093 | 0.1213 | 0.1267 | 0.4822 |
| 1KG | chr6: 162467903 | 0.8594 | 0.0697 | 0.0792 | 0.101 | 0.1113 | 0.8954 |
| 1KG | chr8: 40099493 | 0.98 | 0.1245 | 0.1372 | 0.1725 | 0.1772 | 0.3212 |
| 1KG | rs16822447 | 1.2109 | 0.0633 | 0.0711 | 0.0911 | 0.0976 | 0.7434 |
| 1KG | rs135912 | 1.0272 | 0.0664 | 0.0745 | 0.098 | 0.1047 | 0.6941 |
| 1KG | chr5: 149749881 | 0.9765 | 0.0824 | 0.0917 | 0.1153 | 0.1158 | 0.9777 |
| 1KG | rs2135548 | 1.028 | 0.0466 | 0.0518 | 0.0666 | 0.0695 | 0.9989 |
| 1KG | rs1358395 | 0.9972 | 0.0439 | 0.0494 | 0.0644 | 0.0675 | 0.9751 |
| 1KG | chr3: 126793536 | 0.8615 | 0.1611 | 0.1816 | 0.2392 | 0.2683 | 0.2409 |
| 1KG | rs625761 | 0.8525 | 0.0496 | 0.0554 | 0.074 | 0.0796 | 0.9312 |
| 1KG | rs1540819 | 1.1184 | 0.0407 | 0.0456 | 0.0591 | 0.0622 | 0.9813 |
| 1KG | rs4073997 | 1.2513 | 0.0602 | 0.0674 | 0.0876 | 0.0914 | 0.57 |
| 1KG | chr1: 59476907 | 1.1589 | 0.1439 | 0.1627 | 0.2135 | 0.223 | 0.2323 |
| HM3 | rs16965939 | 1.1299 | 0.0912 | 0.1043 | 0.1383 | 0.151 | 0.6082 |
| HM3 | rs1999930 | 1.0028 | 0.0454 | 0.0509 | 0.0661 | 0.0721 | 0.9854 |
| HM3 | rs1789110 | 1.2138 | 0.0419 | 0.0469 | 0.06 | 0.0635 | 0.9738 |
| HM3 | rs10280782 | 1.2048 | 0.06 | 0.0688 | 0.0859 | 0.093 | 0.9737 |
| HM3 | rs1079982 | 1.1237 | 0.0938 | 0.1057 | 0.1461 | 0.1578 | 0.9122 |
| HM3 | rs11854658 | 1.0274 | 0.0427 | 0.0481 | 0.0624 | 0.0656 | 0.9903 |
| HM3 | rs8076470 | 1.0148 | 0.042 | 0.0469 | 0.0609 | 0.0637 | 0.9768 |
| HM3 | rs8053796 | 0.9076 | 0.0516 | 0.0572 | 0.0738 | 0.0751 | 0.814 |
| HM3 | rs2037156 | 0.9649 | 0.0528 | 0.0586 | 0.0755 | 0.0779 | 0.9516 |
| HM3 | rs3760775 | 1.1136 | 0.0943 | 0.1061 | 0.1445 | 0.1579 | 0.8707 |
| HM3 | rs9291737 | 1.0253 | 0.0413 | 0.0461 | 0.0602 | 0.0638 | 0.9946 |
| HM3 | rs13094238 | 0.724 | 0.0902 | 0.0999 | 0.139 | 0.1451 | 0.4071 |
| HM3 | rs2730613 | 0.9445 | 0.043 | 0.0479 | 0.0633 | 0.0668 | 0.9664 |
| HM3 | rs10103808 | 0.9787 | 0.0682 | 0.0756 | 0.0972 | 0.0992 | 0.9801 |
| HM3 | rs7720497 | 1.0069 | 0.0879 | 0.0973 | 0.1214 | 0.1251 | 0.9157 |
| HM3 | rs6531212 | 0.9223 | 0.0429 | 0.0481 | 0.0627 | 0.0662 | 0.9957 |
| HM3 | rs8011890 | 0.9032 | 0.0813 | 0.0892 | 0.1132 | 0.1141 | 0.76 |
| HM3 | rs12926103 | 1.0152 | 0.0814 | 0.0897 | 0.1148 | 0.115 | 0.9923 |
| HM3 | rs16971055 | 0.9425 | 0.1039 | 0.1156 | 0.1453 | 0.1485 | 0.4562 |
| HM3 | rs1912795 | 1.0147 | 0.0422 | 0.0472 | 0.0613 | 0.0639 | 0.9338 |
| HM3 | rs2127740 | 0.9969 | 0.0621 | 0.0689 | 0.0884 | 0.091 | 0.954 |
| HM3 | rs17155281 | 0.8778 | 0.0558 | 0.0623 | 0.0824 | 0.088 | 0.6339 |
| HM3 | rs4256145 | 0.9604 | 0.0431 | 0.048 | 0.0635 | 0.0675 | 1.0075 |
| HM3 | rs6587759 | 1.1619 | 0.0897 | 0.0977 | 0.1294 | 0.133 | 0.8574 |
| HM3 | rs2052572 | 0.9872 | 0.0434 | 0.0485 | 0.0624 | 0.0653 | 0.9884 |
| HM3 | rs13278062 | 1.2001 | 0.0492 | 0.0547 | 0.0713 | 0.0743 | 0.6759 |
| HM3 | rs722782 | 1.4737 | 0.0689 | 0.0756 | 0.1129 | 0.12 | 1.0109 |
| HM3 | rs1867348 | 0.8191 | 0.0732 | 0.0817 | 0.1008 | 0.1008 | 0.9916 |
| HM3 | rs9461856 | 1.0634 | 0.0405 | 0.0455 | 0.0588 | 0.0614 | 0.993 |
| HM3 | rs2270637 | 0.9701 | 0.0523 | 0.0588 | 0.0774 | 0.0813 | 0.987 |
| HM3 | rs2798832 | 0.9994 | 0.049 | 0.0547 | 0.0699 | 0.0741 | 0.9099 |
| HM3 | rs12632671 | 1.6012 | 0.1014 | 0.1082 | 0.158 | 0.1543 | 0.8674 |
| HM3 | rs9713311 | 1.0534 | 0.0413 | 0.0461 | 0.0595 | 0.0624 | 1.0053 |
| HM3 | rs7626245 | 0.9855 | 0.0776 | 0.0856 | 0.107 | 0.1117 | 0.9805 |
| HM3 | rs513683 | 1.0213 | 0.0412 | 0.0459 | 0.0602 | 0.0629 | 0.977 |
| HM3 | rs2376241 | 0.9227 | 0.0464 | 0.0518 | 0.066 | 0.0686 | 0.9894 |
| HM3 | rs251525 | 1.0694 | 0.0436 | 0.0488 | 0.0626 | 0.0657 | 0.9707 |
| HM3 | rs1737478 | 1.038 | 0.0477 | 0.0531 | 0.0689 | 0.0712 | 0.9949 |
| HM3 | rs7783337 | 1.1463 | 0.0609 | 0.0673 | 0.0854 | 0.0892 | 0.898 |
| HM3 | rs10225927 | 1.009 | 0.05 | 0.0555 | 0.072 | 0.074 | 0.9991 |
| HM3 | rs164700 | 0.9624 | 0.0607 | 0.067 | 0.0861 | 0.0879 | 0.8491 |
| HM3 | rs10848645 | 1.0736 | 0.0455 | 0.0507 | 0.0659 | 0.0685 | 0.7975 |

| IMP | SNP | INFO_1vs5 | INFO_1vs4 | INFO_4vs5 |
|---|---|---|---|---|
| 1KG | rs1367068 | 0.701 | 0.7097 | 0.6902 |
| 1KG | rs1443179 | 0.5865 | 0.5875 | 0.6346 |
| 1KG | rs9328048 | 0.6044 | 0.5908 | 0.6095 |
| 1KG | chr4: 182189551 | 0.3099 | 0.2981 | 0.3154 |
| 1KG | rs1915098 | 0.956 | 0.962 | 0.9292 |
| 1KG | rs1418473 | 0.7416 | 0.7721 | 0.7609 |
| 1KG | rs11183802 | 0.6758 | 0.685 | 0.6375 |
| 1KG | rs728371 | 0.9043 | 0.8945 | 0.9401 |
| 1KG | chr11: 1768072 | 0.7891 | 0.7778 | 0.7614 |
| 1KG | rs11596472 | 0.4412 | 0.4301 | 0.4473 |
| 1KG | rs6982567 | 0.866 | 0.8051 | 0.9272 |
| 1KG | rs2277252 | 0.7457 | 0.7329 | 0.762 |
| 1KG | rs8091635 | 0.7163 | 0.6982 | 0.7201 |
| 1KG | rs9366769 | 0.9573 | 0.9635 | 0.9565 |
| 1KG | rs5771717 | 0.8874 | 0.937 | 0.9215 |
| 1KG | chr22: 33891064 | 0.3013 | 0.3132 | 0.2499 |
| 1KG | chr5: 165133520 | 0.9377 | 0.9432 | 0.9289 |
| 1KG | chr3: 116361297 | 0.5605 | 0.5658 | 0.533 |
| 1KG | rs17071512 | 0.713 | 0.7334 | 0.6406 |
| 1KG | rs1384044 | 0.9636 | 0.9606 | 0.9688 |
| 1KG | rs9308649 | 1.0142 | 1.0187 | 0.9984 |
| 1KG | rs10067691 | 0.5326 | 0.5419 | 0.5069 |
| 1KG | chr1: 100166675 | 0.9053 | 0.9111 | 0.955 |
| 1KG | rs10768315 | 0.9572 | 0.962 | 0.97 |
| 1KG | chr10: 88344492 | 0.9101 | 0.9149 | 0.8994 |
| 1KG | rs7260457 | 0.1968 | 0.1953 | 0.1797 |
| 1KG | chr12: 11233234 | 0.5755 | 0.5496 | 0.6284 |
| 1KG | rs2491146 | 0.7784 | 0.7912 | 0.759 |
| 1KG | chr16: 17310751 | 0.7519 | 0.7696 | 0.7328 |
| 1KG | rs4711751 | 0.7298 | 0.7024 | 0.7775 |
| 1KG | chr11: 104185708 | 0.1747 | 0.1733 | 0.1779 |
| 1KG | rs1851808 | 0.9467 | 0.9713 | 0.9344 |
| 1KG | rs13253938 | 0.8181 | 0.8308 | 0.7808 |
| 1KG | rs529478 | 0.7576 | 0.7498 | 0.7984 |
| 1KG | rs12798294 | 0.8976 | 0.9095 | 0.8898 |
| 1KG | chr8: 123521912 | 0.2511 | 0.2448 | 0.2515 |
| 1KG | rs28621471 | 0.4521 | 0.4409 | 0.4545 |
| 1KG | chr15: 47957127 | 0.5498 | 0.551 | 0.5348 |
| 1KG | chr5: 26212150 | 0.8573 | 0.8674 | 0.874 |
| 1KG | chr2: 180796214 | 0.9684 | 0.9785 | 0.9832 |
| 1KG | rs4967980 | 0.9274 | 0.9152 | 0.915 |
| 1KG | chr6: 119117772 | 0.693 | 0.6975 | 0.6641 |
| 1KG | rs12520598 | 0.6309 | 0.6405 | 0.6062 |
| 1KG | chr10: 56380194 | 0.2728 | 0.258 | 0.307 |
| 1KG | chr12: 8919954 | 0.1946 | 0.1946 | 0.1885 |
| 1KG | rs11592003 | 0.9127 | 0.9064 | 0.8914 |
| 1KG | rs235195 | 0.5723 | 0.5626 | 0.5544 |
| 1KG | rs7623235 | 0.7723 | 0.7628 | 0.7745 |
| 1KG | chr4: 23188024 | 0.7915 | 0.8051 | 0.7724 |
| 1KG | rs12908430 | 0.4692 | 0.4938 | 0.4086 |
| 1KG | chr17: 72483712 | 0.5095 | 0.5126 | 0.5048 |
| 1KG | rs6679773 | 0.1066 | 0.096 | 0.1671 |
| 1KG | chr1: 163986967 | 0.3842 | 0.3754 | 0.3701 |
| 1KG | rs7958529 | 0.7005 | 0.701 | 0.6906 |
| 1KG | chr2: 44132778 | 0.5182 | 0.5121 | 0.5126 |
| 1KG | chr5: 113645446 | 0.8625 | 0.8672 | 0.8696 |
| 1KG | rs11067403 | 0.9691 | 0.963 | 0.9595 |
| 1KG | rs343718 | 0.9727 | 0.9659 | 0.993 |
| 1KG | rs6685751 | 0.4764 | 0.4604 | 0.4831 |
| 1KG | rs1492116 | 1.0053 | 1.0009 | 1.0057 |
| 1KG | rs5015852 | 0.2053 | 0.2065 | 0.1913 |
| 1KG | chr19: 14775139 | 0.5939 | 0.5858 | 0.6019 |
| 1KG | chr11: 124212812 | 0.3285 | 0.3128 | 0.3544 |
| 1KG | rs10503493 | 0.9773 | 0.9755 | 0.9908 |
| 1KG | chr4: 86206508 | 0.4071 | 0.4078 | 0.372 |
| 1KG | rs2883171 | 0.9315 | 0.9061 | 0.9388 |
| 1KG | rs1360751 | 0.977 | 0.9847 | 0.9519 |
| 1KG | rs2024393 | 0.2974 | 0.2803 | 0.3275 |
| 1KG | rs4260755 | 0.8232 | 0.8002 | 0.8358 |
| 1KG | rs17408651 | 0.4856 | 0.4913 | 0.459 |
| 1KG | rs13192030 | 0.3099 | 0.3105 | 0.2907 |
| 1KG | rs12632105 | 0.8939 | 0.9308 | 0.9044 |
| 1KG | chr1: 85757359 | 0.4932 | 0.5197 | 0.4597 |
| 1KG | chr10: 57479107 | 0.9966 | 1.0083 | 1.0117 |
| 1KG | rs7094579 | 0.9967 | 1.0007 | 0.9927 |
| 1KG | chr15: 35201758 | 0.4859 | 0.4789 | 0.4813 |
| 1KG | chr6: 162467903 | 0.9 | 0.9015 | 0.8717 |
| 1KG | chr8: 40099493 | 0.3147 | 0.3094 | 0.3438 |
| 1KG | rs16822447 | 0.75 | 0.7279 | 0.754 |
| 1KG | rs135912 | 0.696 | 0.6876 | 0.6967 |
| 1KG | chr5: 149749881 | 0.9678 | 0.9617 | 0.9997 |
| 1KG | rs2135548 | 1.0021 | 0.9953 | 0.9968 |
| 1KG | rs1358395 | 0.9673 | 0.9727 | 0.9941 |
| 1KG | chr3: 126793536 | 0.2453 | 0.2552 | 0.2102 |
| 1KG | rs625761 | 0.9263 | 0.9461 | 0.9174 |
| 1KG | rs1540819 | 0.984 | 0.9735 | 0.9848 |
| 1KG | rs4073997 | 0.568 | 0.5764 | 0.5644 |
| 1KG | chr1: 59476907 | 0.2253 | 0.2317 | 0.2453 |
| HM3 | rs16965939 | 0.6149 | 0.6524 | 0.5314 |
| HM3 | rs1999930 | 0.9913 | 1.0007 | 0.9517 |
| HM3 | rs1789110 | 0.9672 | 0.9742 | 0.9718 |
| HM3 | rs10280782 | 0.9687 | 0.9696 | 0.987 |
| HM3 | rs1079982 | 0.9093 | 0.9236 | 0.8932 |
| HM3 | rs11854658 | 0.9863 | 0.985 | 0.9979 |
| HM3 | rs8076470 | 0.9766 | 0.9724 | 0.9786 |
| HM3 | rs8053796 | 0.8103 | 0.7844 | 0.8621 |
| HM3 | rs2037156 | 0.9523 | 0.95 | 0.9508 |
| HM3 | rs3760775 | 0.8766 | 0.8826 | 0.8358 |
| HM3 | rs9291737 | 0.997 | 0.9907 | 0.988 |
| HM3 | rs13094238 | 0.4091 | 0.3993 | 0.4129 |
| HM3 | rs2730613 | 0.9713 | 0.9617 | 0.9645 |
| HM3 | rs10103808 | 0.9774 | 0.9757 | 0.9859 |
| HM3 | rs7720497 | 0.9138 | 0.9361 | 0.8956 |
| HM3 | rs6531212 | 0.9989 | 0.9843 | 0.9968 |
| HM3 | rs8011890 | 0.7579 | 0.7236 | 0.7932 |
| HM3 | rs12926103 | 0.9936 | 0.9796 | 1.0029 |
| HM3 | rs16971055 | 0.4484 | 0.4448 | 0.4839 |
| HM3 | rs1912795 | 0.9367 | 0.931 | 0.9339 |
| HM3 | rs2127740 | 0.9591 | 0.9515 | 0.9514 |
| HM3 | rs17155281 | 0.6319 | 0.6427 | 0.6212 |
| HM3 | rs4256145 | 1.0145 | 1.0017 | 0.9959 |
| HM3 | rs6587759 | 0.8617 | 0.8415 | 0.8666 |
| HM3 | rs2052572 | 0.9896 | 0.9903 | 0.981 |
| HM3 | rs13278062 | 0.6707 | 0.6516 | 0.7125 |
| HM3 | rs722782 | 1.0092 | 1.019 | 1.0014 |
| HM3 | rs1867348 | 0.9923 | 0.9708 | 1.0132 |
| HM3 | rs9461856 | 0.9815 | 0.9915 | 1.0084 |
| HM3 | rs2270637 | 0.9813 | 0.9812 | 1.0109 |
| HM3 | rs2798832 | 0.9125 | 0.9197 | 0.8881 |
| HM3 | rs12632671 | 0.8828 | 0.8187 | 0.8905 |
| HM3 | rs9713311 | 1.0066 | 0.9994 | 1.0069 |
| HM3 | rs7626245 | 0.9842 | 0.999 | 0.9526 |
| HM3 | rs513683 | 0.9822 | 0.9615 | 0.9855 |
| HM3 | rs2376241 | 0.9859 | 0.9865 | 1.0007 |
| HM3 | rs251525 | 0.9712 | 0.9706 | 0.9709 |
| HM3 | rs1737478 | 0.9964 | 0.989 | 1.0055 |
| HM3 | rs7783337 | 0.9116 | 0.8804 | 0.8931 |
| HM3 | rs10225927 | 1.0042 | 0.9812 | 1.0102 |
| HM3 | rs164700 | 0.8496 | 0.8256 | 0.8706 |
| HM3 | rs10848645 | 0.7953 | 0.7845 | 0.8209 |

TABLE 5

| SNP | CHR | Gene | POS | EA | OR_4vs5 | P_4vs5 |
|---|---|---|---|---|---|---|
| rs2032794 | 5 | BC034940 | 86468373 | T | 1.382687404 | 9.41E−06 |
| rs7973431 | 12 | BG203966 | 92165329 | T | 1.368256087 | 1.06E−05 |
| rs10207860 | 2 | COL5A2 (alpha 2 type V | 1.9E+08 | T | 0.598765047 | 8.56E−06 |
| | | collagen preprotein) | | | | |
| rs11207037 | 1 | DAB1 | 57677552 | A | 1.351136378 | 2.44E−05 |
| rs4771513 | 13 | downstream AK058053 | 1.05E+08 | T | 1.55674951 | 8.13E−06 |
| rs2469875 | 18 | downstream KC6 | 36481096 | A | 0.769170975 | 2.42E−05 |
| rs16924889 | 10 | downstream KIAA1217 | 24883792 | A | 1.694071834 | 2.95E−05 |
| rs11876415 | 18 | NETO1 | 68641554 | T | 0.693451484 | 8.51E−06 |
| rs5771717 | 22 | FAM19A5 | 47467676 | A | 0.627530502 | 1.35E−06 |
| rs17639345 | 4 | FSTL5 | 1.63E+08 | A | 1.478590102 | 1.81E−05 |
| rs3743105 | 15 | GREM1 | 30811243 | T | 0.7675482 | 2.62E−05 |
| rs11716363 | 3 | ITGA9 (integrin alpha 9 | 37504640 | T | 1.443900309 | 1.66E−05 |
| | | precursor) | | | | |
| rs11736266 | 4 | MARCH1 gene | 1.65E+08 | T | 1.302693443 | 2.45E−05 |
| rs7317763 | 13 | near SLITRK1 | 83069357 | T | 1.310328671 | 2.70E−05 |
| rs9475939 | 6 | PRIM2 | 57396889 | T | 2.48739008 | 1.30E−05 |
| rs4755455 | 11 | upstream QSER1 | 32859894 | C | 1.579504199 | 4.64E−07 |
| rs10815017 | 9 | SLC1A1 | 4536594 | T | 0.733460158 | 6.75E−06 |
| rs1326005 | 1 | upstream AK125078 | 5303445 | A | 0.729567782 | 3.42E−06 |
| rs7973032 | 12 | upstream PPFIA2 | 80847264 | A | 0.539440385 | 1.67E−05 |
| rs958646 | 20 | upstream PRNP | 4486243 | C | 0.640698687 | 3.05E−05 |

TABLE 6

| REF | hg_18_BP | OldSNP | A1 | A2 | FRQ_A(2594) | FRQ_U(4134) | OR |
|---|---|---|---|---|---|---|---|
| 1KG | 1:100166675 | chr1: 100166675 | T | C | 0.0493 | 0.0366 | 1.3684 |
| 1KG | 1:163986967 | chr1: 163986967 | T | C | 0.052 | 0.059 | 0.7049 |
| 1KG | 1:165795961 | rs1737478 | T | C | 0.2451 | 0.2262 | 1.1357 |
| 1KG | 1:185513773 | rs2453727 | T | C | 0.4977 | 0.492 | 1.0017 |
| 1KG | 1:194925860 | rs1061170 | T | C | 0.3909 | 0.6287 | 0.3651 |
| 1KG | 1:194963556 | rs1410996 | A | G | 0.2004 | 0.4206 | 0.3207 |
| 1KG | 1:205461564 | rs1367068 | A | G | 0.8012 | 0.774 | 1.4018 |
| 1KG | 1:205515927 | rs12040406 | T | C | 0.9174 | 0.906 | 2.1921 |
| 1KG | 1:227122574 | rs6685751 | A | C | 0.4818 | 0.4657 | 1.1607 |
| 1KG | 1:228362314 | rs4846914 | A | G | 0.5938 | 0.6043 | 0.9609 |
| 1KG | 1:247130153 | rs6587759 | A | G | 0.0635 | 0.051 | 1.3373 |
| 1KG | 1:31101812 | rs2491146 | A | G | 0.3668 | 0.3933 | 0.8641 |
| 1KG | 1:5303445 | rs1326005 | A | G | 0.3841 | 0.4049 | 0.9191 |
| 1KG | 1:57219300 | rs1418473 | A | G | 0.0866 | 0.072 | 1.221 |
| 1KG | 1:57677552 | rs11207037 | A | G | 0.7507 | 0.7569 | 0.9771 |
| 1KG | 1:79360392 | rs17408651 | T | G | 0.1174 | 0.1288 | 0.8501 |
| 1KG | 10:124204438 | rs10490924 | T | G | 0.4144 | 0.2058 | 3.1865 |
| 1KG | 10:19392088 | rs11596472 | A | G | 0.9141 | 0.9174 | 0.869 |
| 1KG | 10:24883792 | rs16924889 | A | G | 0.0865 | 0.0931 | 0.9404 |
| 1KG | 10:32036318 | rs2277252 | A | G | 0.4765 | 0.455 | 1.1865 |
| 1KG | 10:3945290 | rs11592003 | T | C | 0.8623 | 0.8385 | 1.2203 |
| 1KG | 10:56380194 | rs61856267 | A | G | 0.8865 | 0.9054 | 0.7381 |
| 1KG | 10:57479107 | chr10: 57479107 | A | C | 0.1191 | 0.1054 | 1.1634 |
| 1KG | 10:8226099 | rs2798832 | T | C | 0.266 | 0.2403 | 1.166 |
| 1KG | 10:88344492 | chr10: 88344492 | A | C | 0.1597 | 0.1406 | 1.2144 |
| 1KG | 10:88396729 | rs2803544 | A | T | 0.1528 | 0.1335 | 1.2022 |
| 1KG | 10:92382787 | rs7094579 | T | C | 0.0451 | 0.0568 | 0.753 |
| 1KG | 11:100149779 | rs625761 | A | G | 0.7619 | 0.7375 | 1.1585 |
| 1KG | 11:103362906 | rs12798294 | C | G | 0.2689 | 0.2959 | 0.8706 |
| 1KG | 11:104185708 | chr11: 104185708 | T | G | 0.0478 | 0.0463 | 1.7253 |
| 1KG | 11:116154127 | rs964184 | C | G | 0.862 | 0.8567 | 1.0379 |
| 1KG | 11:1762440 | rs55911157 | T | C | 0.1489 | 0.1731 | 0.7781 |
| 1KG | 11:1768072 | rs72850977 | A | G | 0.148 | 0.1723 | 0.7792 |
| 1KG | 11:32859894 | rs4755455 | C | G | 0.1684 | 0.1757 | 0.9526 |
| 1KG | 11:37597461 | rs10768315 | T | C | 0.2642 | 0.2971 | 0.8472 |
| 1KG | 11:61327359 | rs174547 | T | C | 0.666 | 0.6552 | 1.05 |
| 1KG | 11:73662041 | rs513683 | C | G | 0.4409 | 0.4611 | 0.8991 |
| 1KG | 12:108379551 | rs2338104 | C | G | 0.4627 | 0.4579 | 1.046 |
| 1KG | 12:11233234 | chr12: 11233234 | A | T | 0.1532 | 0.1392 | 1.1565 |
| 1KG | 12:114010367 | rs11067403 | T | C | 0.6891 | 0.658 | 1.1478 |
| 1KG | 12:123850701 | rs5888 | A | G | 0.4889 | 0.4859 | 0.998 |
| 1KG | 12:19878442 | rs1492116 | T | C | 0.79 | 0.7665 | 1.1876 |
| 1KG | 12:2290505 | rs10848645 | A | G | 0.571 | 0.6031 | 0.8399 |
| 1KG | 12:45893566 | rs11183802 | A | G | 0.0774 | 0.0708 | 1.3253 |
| 1KG | 12:50881293 | rs7958529 | C | G | 0.6738 | 0.6506 | 1.1835 |
| 1KG | 12:80847264 | rs7973032 | A | G | 0.9291 | 0.9315 | 1.0429 |
| 1KG | 12:92165329 | rs7973431 | T | C | 0.3164 | 0.3168 | 1.0087 |
| 1KG | 13:105365389 | rs4771513 | T | C | 0.8738 | 0.8725 | 1.053 |
| 1KG | 13:30736688 | rs1912795 | A | G | 0.4579 | 0.4329 | 1.1392 |
| 1KG | 13:78960018 | rs17071512 | T | C | 0.9039 | 0.8967 | 1.2417 |
| 1KG | 13:83069357 | rs7317763 | T | C | 0.4613 | 0.4456 | 1.0722 |
| 1KG | 14:95216646 | rs8011890 | A | C | 0.099 | 0.0828 | 1.4427 |
| 1KG | 15:30811243 | rs3743105 | T | C | 0.4142 | 0.4211 | 0.9607 |
| 1KG | 15:35201758 | chr15: 35201758 | T | G | 0.1508 | 0.1344 | 1.3375 |
| 1KG | 15:35992085 | rs16965939 | T | C | 0.0907 | 0.1081 | 0.6687 |
| 1KG | 15:40375218 | rs12908430 | A | G | 0.0812 | 0.0899 | 0.6083 |
| 1KG | 15:47957127 | chr15: 47957127 | T | G | 0.0105 | 0.0115 | 0.1281 |
| 1KG | 15:56465804 | rs10468017 | T | C | 0.2564 | 0.2874 | 0.8324 |
| 1KG | 15:56475172 | rs493258 | T | C | 0.4318 | 0.4714 | 0.8481 |
| 1KG | 15:59828725 | rs11854497 | A | G | 0.1036 | 0.1139 | 0.825 |
| 1KG | 15:87312556 | rs11854658 | A | G | 0.6764 | 0.6461 | 1.1633 |
| 1KG | 15:87314645 | rs11856826 | T | C | 0.6577 | 0.6267 | 1.1722 |
| 1KG | 16:11814579 | rs2024393 | T | C | 0.16 | 0.1583 | 1.0619 |
| 1KG | 16:17310751 | chr16: 17310751 | A | G | 0.9164 | 0.8991 | 1.3105 |
| 1KG | 16:23091095 | rs4967980 | T | C | 0.7435 | 0.7747 | 0.8305 |
| 1KG | 16:55550825 | rs3764261 | A | C | 0.36 | 0.3277 | 1.1631 |
| 1KG | 16:66459571 | rs2271293 | A | G | 0.1249 | 0.1157 | 1.0897 |
| 1KG | 16:72289810 | rs2127740 | A | G | 0.1392 | 0.1138 | 1.2894 |
| 1KG | 16:74921678 | rs8053796 | T | C | 0.2789 | 0.2638 | 1.1928 |
| 1KG | 16:74929641 | rs6564324 | A | G | 0.7163 | 0.7325 | 0.8354 |
| 1KG | 16:84929276 | rs12926103 | A | G | 0.0664 | 0.0573 | 1.4308 |
| 1KG | 16:85846518 | rs3748391 | T | G | 0.518 | 0.5459 | 0.8754 |
| 1KG | 17:45980827 | rs8076470 | T | C | 0.4062 | 0.3844 | 1.1472 |
| 1KG | 17:72483712 | chr17: 72483712 | A | G | 0.9454 | 0.9428 | 1.2989 |
| 1KG | 17:73838135 | rs16971055 | A | G | 0.096 | 0.0876 | 1.3213 |
| 1KG | 17:77136846 | rs4073997 | C | G | 0.212 | 0.2118 | 1.1002 |
| 1KG | 18:34111545 | rs8091635 | T | C | 0.3871 | 0.3485 | 1.1987 |
| 1KG | 18:34125717 | rs17628762 | A | C | 0.3824 | 0.3426 | 1.188 |
| 1KG | 18:36481096 | rs2469875 | A | G | 0.5168 | 0.5163 | 1.0045 |
| 1KG | 18:45421212 | rs4939883 | T | C | 0.1743 | 0.1714 | 1.0146 |
| 1KG | 18:53934507 | rs529478 | A | T | 0.5166 | 0.4897 | 1.2005 |
| 1KG | 18:68641554 | rs11876415 | T | C | 0.6298 | 0.6297 | 1.0636 |
| 1KG | 18:72988032 | rs1789110 | A | C | 0.5878 | 0.6251 | 0.8321 |
| 1KG | 19:36639376 | rs2052572 | A | G | 0.3482 | 0.3141 | 1.1729 |
| 1KG | 19:5792356 | rs3760775 | T | G | 0.055 | 0.0679 | 0.7243 |
| HM2 | 19:59425996 | rs7260457 | C | G | 0.3679 | 0.3736 | 0.7477 |
| 1KG | 19:6669387 | rs2230199 | C | G | 0.2439 | 0.1926 | 1.6842 |
| 1KG | 19:8375738 | rs2967605 | T | C | 0.1811 | 0.1762 | 1.0509 |
| 1KG | 2:124751486 | rs9308649 | A | T | 0.8153 | 0.7908 | 1.198 |
| 1KG | 2:180796214 | chr2: 180796214 | A | C | 0.1728 | 0.1957 | 0.8419 |
| 1KG | 2:189854831 | rs10207860 | T | C | 0.0682 | 0.0719 | 0.9 |
| 1KG | 2:20201501 | rs6531212 | T | C | 0.3177 | 0.3514 | 0.8491 |
| 1KG | 2:44132778 | chr2: 44132778 | A | G | 0.9374 | 0.9244 | 1.4611 |
| 1KG | 2:71597893 | rs13402171 | T | C | 0.0904 | 0.091 | 1.0004 |
| 1KG | 2:76232489 | rs1851808 | A | G | 0.6074 | 0.6375 | 0.8696 |
| 1KG | 20:42475778 | rs1800961 | T | C | 0.0264 | 0.0314 | 0.8286 |
| 1KG | 20:44009909 | rs7679 | T | C | 0.8196 | 0.8135 | 1.0601 |
| 1KG | 20:4486243 | rs958646 | C | G | 0.0838 | 0.087 | 0.9867 |
| 1KG | 20:56979160 | rs235195 | A | G | 0.1336 | 0.1251 | 1.364 |
| HM2 | 22:31414511 | rs9621532 | A | C | 0.9634 | 0.9488 | 1.3892 |
| 1KG | 22:36259705 | rs1079982 | T | G | 0.0471 | 0.065 | 0.7054 |
| 1KG | 22:43172593 | rs135912 | A | G | 0.7348 | 0.7265 | 1.0507 |
| 1KG | 22:47467676 | rs5771717 | A | G | 0.1034 | 0.1002 | 1.1586 |
| 1KG | 3:100878962 | rs13095226 | T | C | 0.8772 | 0.896 | 0.8167 |
| 1KG | 3:101053451 | rs7626245 | C | G | 0.0904 | 0.0727 | 1.3399 |
| 1KG | 3:118210505 | rs1540819 | T | C | 0.4652 | 0.4345 | 1.1303 |
| 1KG | 3:119674993 | rs12637095 | A | T | 0.8045 | 0.7797 | 1.1955 |
| 1KG | 3:119681130 | rs1915098 | A | G | 0.1414 | 0.1655 | 0.7983 |
| 1KG | 3:120558458 | rs9713311 | T | C | 0.5952 | 0.6235 | 0.8814 |
| 1KG | 3:130591125 | rs12632105 | C | G | 0.1669 | 0.1695 | 0.9737 |
| 1KG | 3:154434339 | rs16822447 | A | T | 0.8637 | 0.8442 | 1.178 |
| 1KG | 3:156261403 | rs13094238 | T | C | 0.8604 | 0.8539 | 1.3416 |
| 1KG | 3:163192835 | rs4256145 | A | G | 0.6945 | 0.6619 | 1.185 |
| 1KG | 3:37504640 | rs11716363 | T | C | 0.1892 | 0.179 | 1.0861 |
| 1KG | 3:53233464 | rs12632671 | A | G | 0.0676 | 0.0568 | 1.4031 |
| 1KG | 3:71584585 | rs2135548 | A | G | 0.7302 | 0.7598 | 0.8518 |
| 1KG | 4:110878516 | rs10033900 | T | C | 0.5205 | 0.463 | 1.3144 |
| 1KG | 4:128495772 | rs1443179 | T | G | 0.0388 | 0.0348 | 1.7594 |
| 1KG | 4:162971300 | rs17639345 | A | G | 0.1539 | 0.1466 | 1.0405 |
| 1KG | 4:165142286 | rs11736266 | T | C | 0.478 | 0.4827 | 0.951 |
| 1KG | 4:182181154 | rs9997006 | T | G | 0.3841 | 0.3654 | 1.2106 |
| 1KG | 4:23188024 | chr4: 23188024 | A | T | 0.0345 | 0.0389 | 0.491 |
| 1KG | 4:36840346 | rs2376241 | T | G | 0.2687 | 0.2423 | 1.148 |
| 1KG | 4:86877248 | rs28621471 | A | G | 0.2253 | 0.2481 | 0.8532 |
| 1KG | 5:106936632 | rs164700 | A | C | 0.1655 | 0.142 | 1.2085 |
| 1KG | 5:113645446 | chr5: 113645446 | C | G | 0.8488 | 0.868 | 0.8275 |
| 1KG | 5:149749881 | chr5: 149749881 | C | G | 0.0812 | 0.0713 | 1.2083 |
| 1KG | 5:165133520 | chr5: 165133520 | T | C | 0.8269 | 0.8052 | 1.1637 |
| 1KG | 5:21309600 | rs2883171 | T | G | 0.863 | 0.884 | 0.7562 |
| 1KG | 5:26212150 | chr5: 26212150 | T | C | 0.9433 | 0.9267 | 1.4173 |
| 1KG | 5:52621268 | rs251525 | A | G | 0.6677 | 0.6899 | 0.8717 |
| 1KG | 5:5291813 | rs7720497 | C | G | 0.932 | 0.9454 | 0.7282 |
| 1KG | 5:61112540 | rs9291737 | A | T | 0.6143 | 0.5789 | 1.1581 |
| 1KG | 5:64292165 | rs10067691 | A | G | 0.0525 | 0.0594 | 0.694 |
| 1KG | 5:74385314 | rs12520598 | T | C | 0.0438 | 0.0509 | 0.7993 |
| 1KG | 5:86468373 | rs2032794 | T | C | 0.7796 | 0.7892 | 0.9488 |
| 1KG | 6:109470664 | rs13192030 | A | T | 0.9634 | 0.9643 | 1.3742 |
| 1KG | 6:116493827 | rs1999930 | T | C | 0.2595 | 0.3044 | 0.8068 |
| 1KG | 6:116529937 | rs509859 | T | G | 0.3742 | 0.4133 | 0.8508 |
| 1KG | 6:116568331 | rs12204816 | T | C | 0.1906 | 0.224 | 0.7488 |
| 1KG | 6:116596243 | rs12196141 | A | G | 0.7543 | 0.7128 | 1.254 |
| 1KG | 6:1324870 | rs9328048 | T | C | 0.3673 | 0.395 | 0.846 |
| 1KG | 6:132523980 | rs728371 | A | G | 0.3437 | 0.3145 | 1.1821 |
| 1KG | 6:160376153 | rs1867348 | T | C | 0.0986 | 0.0801 | 1.2525 |
| 1KG | 6:162467903 | chr6: 162467903 | A | G | 0.0781 | 0.09 | 0.8001 |
| 1KG | 6:31277268 | rs9366769 | T | C | 0.8016 | 0.7695 | 1.2329 |
| 1KG | 6:32011783 | rs9332739 | C | G | 0.0231 | 0.0447 | 0.4463 |
| 1KG | 6:32022159 | rs641153 | A | G | 0.0528 | 0.101 | 0.461 |
| 1KG | 6:33503177 | rs9461856 | A | G | 0.4878 | 0.5204 | 0.8641 |
| 1KG | 6:43936560 | rs4711751 | T | C | 0.5352 | 0.5044 | 1.2081 |
| 1KG | 6:55456674 | rs4260755 | A | C | 0.3862 | 0.3512 | 1.152 |
| 1KG | 6:57396889 | rs9475939 | T | C | 0.9717 | 0.9716 | 0.7707 |
| 1KG | 6:7063989 | rs11755724 | A | G | 0.3424 | 0.3723 | 0.878 |
| 1KG | 6:7152544 | rs1360751 | A | C | 0.5508 | 0.5208 | 1.1507 |
| 1KG | 6:93573222 | rs2037156 | A | G | 0.7857 | 0.8074 | 0.8562 |
| HM2 | 7:14123511 | rs7783337 | A | G | 0.8481 | 0.8625 | 0.8475 |
| 1KG | 7:23550807 | rs10280782 | A | G | 0.8768 | 0.8487 | 1.3025 |
| 1KG | 7:27475356 | rs17155281 | T | C | 0.724 | 0.7083 | 1.1382 |
| 1KG | 7:35595154 | rs343718 | A | C | 0.2884 | 0.3029 | 0.9357 |
| 1KG | 7:43880667 | rs2730613 | A | C | 0.6656 | 0.6475 | 1.1333 |
| 1KG | 7:54736904 | rs10225927 | A | G | 0.7841 | 0.8095 | 0.847 |
| 1KG | 7:86076557 | rs1358395 | A | G | 0.2901 | 0.3185 | 0.8507 |
| 1KG | 8:123521501 | rs4282579 | A | G | 0.6556 | 0.6888 | 0.8571 |
| 1KG | 8:128550166 | rs10089310 | A | T | 0.1149 | 0.0961 | 1.2349 |
| 1KG | 8:136110326 | rs13253938 | A | G | 0.4722 | 0.4365 | 1.1837 |
| 1KG | 8:13715406 | rs1384044 | T | C | 0.5042 | 0.5311 | 0.8777 |
| 1KG | 8:139212254 | rs10103808 | A | T | 0.8852 | 0.9095 | 0.754 |
| 1KG | 8:14247492 | rs10503493 | A | C | 0.0425 | 0.055 | 0.7705 |
| 1KG | 8:19888502 | rs12678919 | A | G | 0.9011 | 0.899 | 1.0259 |
| 1KG | 8:20081107 | rs2270637 | C | G | 0.8272 | 0.8001 | 1.222 |
| 1KG | 8:23138916 | rs13278062 | T | G | 0.5369 | 0.5138 | 1.1804 |
| 1KG | 8:506479 | rs722782 | A | C | 0.0841 | 0.1055 | 0.7698 |
| HM2 | 8:96819457 | rs6982567 | T | C | 0.2077 | 0.1912 | 1.1905 |
| 1KG | 9:106704122 | rs1883025 | T | C | 0.2564 | 0.2784 | 0.8697 |
| 1KG | 9:114121319 | rs10739343 | A | G | 0.2015 | 0.2256 | 0.8504 |
| 1KG | 9:15279578 | rs471364 | T | C | 0.8882 | 0.8857 | 1.0291 |
| 1KG | 9:4536594 | rs10815017 | T | C | 0.2991 | 0.3153 | 0.9327 |

| REF | hg_18_BP | OldSNP | SE | P | EA | Z |
|---|---|---|---|---|---|---|
| 1KG | 1:100166675 | chr1: 100166675 | 0.0913 | 0.0005929 | T | 3.434841729 |
| 1KG | 1:163986967 | chr1: 163986967 | 0.1261 | 0.00555 | T | −2.773246868 |
| 1KG | 1:165795961 | rs1737478 | 0.0437 | 0.003573 | T | 2.913588976 |
| 1KG | 1:185513773 | rs2453727 | 0.0371 | 0.964 | T | 0.045134628 |
| 1KG | 1:194925860 | rs1061170 | 0.0403 | 5.638E−138 | C | 25.00323274 |
| 1KG | 1:194963556 | rs1410996 | 0.0461 | 2.147E−134 | G | 24.67181558 |
| 1KG | 1:205461564 | rs1367068 | 0.0658 | 2.796E−07 | G | −5.136703203 |
| 1KG | 1:205515927 | rs12040406 | 0.1528 | 2.786E−07 | C | −5.137376796 |
| 1KG | 1:227122574 | rs6685751 | 0.0507 | 0.003299 | A | 2.9384104 |
| 1KG | 1:228362314 | rs4846914 | 0.038 | 0.2946 | G | 1.048083799 |
| 1KG | 1:247130153 | rs6587759 | 0.0817 | 0.0003756 | A | 3.556659831 |
| 1KG | 1:31101812 | rs2491146 | 0.0409 | 0.000358 | A | −3.569253417 |
| 1KG | 1:5303445 | rs1326005 | 0.0404 | 0.03688 | A | −2.087089998 |
| 1KG | 1:57219300 | rs1418473 | 0.0714 | 0.005179 | A | 2.795684072 |
| 1KG | 1:57677552 | rs11207037 | 0.0436 | 0.5953 | G | 0.531171414 |
| 1KG | 1:79360392 | rs17408651 | 0.0666 | 0.01478 | T | −2.437725319 |
| 1KG | 10:124204438 | rs10490924 | 0.0453 | 1.21E−144 | T | 25.60903658 |
| 1KG | 10:19392088 | rs11596472 | 0.1042 | 0.1779 | G | 1.34724916 |
| 1KG | 10:24883792 | rs16924889 | 0.0679 | 0.3655 | A | −0.904934667 |
| 1KG | 10:32036318 | rs2277252 | 0.0437 | 0.00008953 | A | 3.917343855 |
| 1KG | 10:3945290 | rs11592003 | 0.0533 | 0.0001869 | C | −3.736095111 |
| 1KG | 10:56380194 | rs61856267 | 0.0745 | 0.00004618 | G | 4.074163657 |
| 1KG | 10:57479107 | chr10: 57479107 | 0.0593 | 0.01073 | A | 2.551371123 |
| 1KG | 10:8226099 | rs2798832 | 0.0444 | 0.0005466 | T | 3.456814979 |
| 1KG | 10:88344492 | chr10: 88344492 | 0.0534 | 0.0002725 | A | 3.640130402 |
| 1KG | 10:88396729 | rs2803544 | 0.0536 | 0.000592 | A | 3.435253392 |
| 1KG | 10:92382787 | rs7094579 | 0.0857 | 0.0009303 | T | −3.310799016 |
| 1KG | 11:100149779 | rs625761 | 0.0444 | 0.0009244 | G | −3.312579045 |
| 1KG | 11:103362906 | rs12798294 | 0.0421 | 0.0009907 | C | −3.293154553 |
| 1KG | 11:104185708 | chr11: 104185708 | 0.2201 | 0.01322 | T | 2.477787107 |
| 1KG | 11:116154127 | rs964184 | 0.0538 | 0.4885 | G | −0.692696557 |
| 1KG | 11:1762440 | rs55911157 | 0.0567 | 0.000009621 | T | −4.425520544 |
| 1KG | 11:1768072 | rs72850977 | 0.0562 | 0.000008946 | A | −4.441196633 |
| 1KG | 11:32859894 | rs4755455 | 0.0498 | 0.3297 | C | −0.974718326 |
| 1KG | 11:37597461 | rs10768315 | 0.0417 | 0.00007111 | T | −3.972540972 |
| 1KG | 11:61327359 | rs174547 | 0.039 | 0.2107 | C | −1.251642941 |
| 1KG | 11:73662041 | rs513683 | 0.0386 | 0.005903 | C | −2.753121412 |
| 1KG | 12:108379551 | rs2338104 | 0.0372 | 0.2264 | G | −1.209684447 |
| 1KG | 12:11233234 | chr12: 11233234 | 0.0716 | 0.04241 | A | 2.02947434 |
| 1KG | 12:114010367 | rs11067403 | 0.0396 | 0.0005053 | C | −3.477930989 |
| 1KG | 12:123850701 | rs5888 | 0.0387 | 0.9593 | G | 0.051032027 |
| 1KG | 12:19878442 | rs1492116 | 0.0449 | 0.00013 | C | −3.826458602 |
| 1KG | 12:2290505 | rs10848645 | 0.0435 | 0.00006143 | G | 4.007249345 |
| 1KG | 12:45893566 | rs11183802 | 0.0906 | 0.001883 | A | 3.108091123 |
| 1KG | 12:50881293 | rs7958529 | 0.044 | 0.0001293 | G | −3.827788094 |
| 1KG | 12:80847264 | rs7973032 | 0.075 | 0.5758 | G | −0.559530092 |
| 1KG | 12:92165329 | rs7973431 | 0.0412 | 0.8331 | T | 0.21072739 |
| 1KG | 13:105365389 | rs4771513 | 0.0594 | 0.3845 | C | −0.869634825 |
| 1KG | 13:30736688 | rs1912795 | 0.0379 | 0.0005884 | A | 3.436905894 |
| 1KG | 13:78960018 | rs17071512 | 0.0726 | 0.002856 | C | −2.982826333 |
| 1KG | 13:83069357 | rs7317763 | 0.0378 | 0.06489 | T | 1.846015189 |
| 1KG | 14:95216646 | rs8011890 | 0.0881 | 0.00003215 | A | 4.157677112 |
| 1KG | 15:30811243 | rs3743105 | 0.0381 | 0.293 | T | −1.051563198 |
| 1KG | 15:35201758 | chr15: 35201758 | 0.069 | 0.00002486 | T | 4.216066935 |
| 1KG | 15:35992085 | rs16965939 | 0.0934 | 0.00001622 | T | −4.311432897 |
| 1KG | 15:40375218 | rs12908430 | 0.1414 | 0.0004389 | A | −3.515516189 |
| 1KG | 15:47957127 | chr15: 47957127 | 0.9639 | 0.03299 | T | −2.132204971 |
| 1KG | 15:56465804 | rs10468017 | 0.045 | 0.00004595 | T | −4.075325644 |
| 1KG | 15:56475172 | rs493258 | 0.0376 | 0.00001198 | C | 4.377951501 |
| 1KG | 15:59828725 | rs11854497 | 0.0618 | 0.001868 | A | −3.110453982 |
| 1KG | 15:87312556 | rs11854658 | 0.0391 | 0.0001108 | G | −3.865636189 |
| 1KG | 15:87314645 | rs11856826 | 0.0406 | 0.00009204 | C | −3.910671187 |
| 1KG | 16:11814579 | rs2024393 | 0.0665 | 0.3666 | T | 0.902860381 |
| 1KG | 16:17310751 | chr16: 17310751 | 0.0695 | 0.0001012 | G | −3.887696628 |
| 1KG | 16:23091095 | rs4967980 | 0.045 | 0.0000362 | C | 4.130484184 |
| 1KG | 16:55550825 | rs3764261 | 0.0392 | 0.0001162 | A | 3.85400757 |
| 1KG | 16:66459571 | rs2271293 | 0.0574 | 0.1345 | A | 1.496589899 |
| 1KG | 16:72289810 | rs2127740 | 0.057 | 0.000008169 | A | 4.460706246 |
| 1KG | 16:74921678 | rs8053796 | 0.048 | 0.0002414 | T | 3.671215171 |
| 1KG | 16:74929641 | rs6564324 | 0.0481 | 0.0001849 | G | 3.738801021 |
| 1KG | 16:84929276 | rs12926103 | 0.0864 | 0.00003384 | A | 4.145955195 |
| 1KG | 16:85846518 | rs3748391 | 0.037 | 0.0003254 | T | −3.594191857 |
| 1KG | 17:45980827 | rs8076470 | 0.0392 | 0.0004619 | T | 3.501930552 |
| 1KG | 17:72483712 | chr17: 72483712 | 0.1484 | 0.07803 | G | −1.762232637 |
| 1KG | 17:73838135 | rs16971055 | 0.0998 | 0.00524 | A | 2.791897205 |
| 1KG | 17:77136846 | rs4073997 | 0.1728 | 0.5804 | C | 0.552800538 |
| 1KG | 18:34111545 | rs8091635 | 0.0401 | 0.000006243 | T | 4.517988076 |
| 1KG | 18:34125717 | rs17628762 | 0.0388 | 0.000008979 | A | 4.440404368 |
| 1KG | 18:36481096 | rs2469875 | 0.037 | 0.9027 | G | −0.122251299 |
| 1KG | 18:45421212 | rs4939883 | 0.0486 | 0.7654 | T | 0.298397234 |
| 1KG | 18:53934507 | rs529478 | 0.0449 | 0.00004656 | T | −4.072255823 |
| 1KG | 18:68641554 | rs11876415 | 0.0486 | 0.205 | C | −1.267434417 |
| 1KG | 18:72988032 | rs1789110 | 0.0394 | 0.000003171 | C | 4.659421045 |
| 1KG | 19:36639376 | rs2052572 | 0.0409 | 0.00009603 | A | 3.9004098 |
| 1KG | 19:5792356 | rs3760775 | 0.0866 | 0.0001966 | T | −3.723345742 |
| HM2 | 19:59425996 | rs7260457 | 0.1028 | 0.004664 | C | −2.829368242 |
| 1KG | 19:6669387 | rs2230199 | 0.0592 | 1.357E−18 | C | 8.800919225 |
| 1KG | 19:8375738 | rs2967605 | 0.0505 | 0.3256 | T | 0.9830151.14 |
| 1KG | 2:124751486 | rs9308649 | 0.0476 | 0.0001473 | T | −3.795577787 |
| 1KG | 2:180796214 | chr2: 180796214 | 0.0503 | 0.0006251 | A | −3.420483185 |
| 1KG | 2:189854831 | rs10207860 | 0.0765 | 0.1687 | T | −1.376392981 |
| 1KG | 2:20201501 | rs6531212 | 0.0392 | 0.00003021 | T | −4.171877535 |
| 1KG | 2:44132778 | chr2:44132778 | 0.1037 | 0.0002569 | G | −3.655281432 |
| 1KG | 2:71597893 | rs13402171 | 0.064 | 0.9956 | T | 0.00551461 |
| 1KG | 2:76232489 | rs1851808 | 0.039 | 0.0003417 | G | 3.581444295 |
| 1KG | 20:42475778 | rs1800961 | 0.112 | 0.09321 | T | −1.678702697 |
| 1KG | 20:44009909 | rs7679 | 0.0483 | 0.227 | C | −1.20812288 |
| 1KG | 20:4486243 | rs958646 | 0.0671 | 0.8419 | C | −0.199463746 |
| 1KG | 20:56979160 | rs235195 | 0.104 | 0.002827 | A | 2.985948769 |
| HM2 | 22:31414511 | rs9621532 | 0.0924 | 0.0003732 | C | −3.558344238 |
| 1KG | 22:36259705 | rs1079982 | 0.0923 | 0.0001557 | T | −3.781797499 |
| 1KG | 22:43172593 | rs135912 | 0.0492 | 0.3149 | G | −1.004993458 |
| 1KG | 22:47467676 | rs5771717 | 0.0896 | 0.1004 | A | 1.642917518 |
| 1KG | 3:100878962 | rs13095226 | 0.0579 | 0.0004761 | C | 3.493854735 |
| 1KG | 3:101053451 | rs7626245 | 0.0712 | 0.00003955 | C | 4.110092882 |
| 1KG | 3:118210505 | rs1540819 | 0.0379 | 0.001249 | T | 3.227447405 |
| 1KG | 3:119674993 | rs12637095 | 0.0462 | 0.0001092 | T | −3.869184471 |
| 1KG | 3:119681130 | rs1915098 | 0.0533 | 0.00002407 | A | −4.223347742 |
| 1KG | 3:120558458 | rs9713311 | 0.0375 | 0.0007681 | C | 3.364041473 |
| 1KG | 3:130591125 | rs12632105 | 0.0552 | 0.6286 | C | −0.4836983 |
| 1KG | 3:154434339 | rs16822447 | 0.055 | 0.002918 | T | −2.976246725 |
| 1KG | 3:156261403 | rs13094238 | 0.1084 | 0.006705 | C | −2.711151416 |
| 1KG | 3:163192835 | rs4256145 | 0.04 | 0.00002249 | G | −4.238617964 |
| 1KG | 3:37504640 | rs11716363 | 0.0509 | 0.105 | T | 1.621082251 |
| 1KG | 3:53233464 | rs12632671 | 0.0924 | 0.0002466 | A | 3.665765312 |
| 1KG | 3:71584585 | rs2135548 | 0.0422 | 0.0001424 | G | 3.803962059 |
| 1KG | 4:110878516 | rs10033900 | 0.0409 | 2.401E−11 | T | 6.679291601 |
| 1KG | 4:128495772 | rs1443179 | 0.1788 | 0.001578 | T | 3.159943013 |
| 1KG | 4:162971300 | rs17639345 | 0.0515 | 0.4416 | A | 0.769494177 |
| 1KG | 4:165142286 | rs11736266 | 0.0392 | 0.1999 | C | 1.281836521 |
| 1KG | 4:182181154 | rs9997006 | 0.0571 | 0.0008115 | T | 3.3488422 |
| 1KG | 4:23188024 | chr4: 23188024 | 0.2341 | 0.002383 | A | −3.037815254 |
| 1KG | 4:36840346 | rs2376241 | 0.0425 | 0.001166 | T | 3.247069288 |
| 1KG | 4:86877248 | rs28621471 | 0.0475 | 0.0008447 | A | −3.337716127 |
| 1KG | 5:106936632 | rs164700 | 0.0614 | 0.002048 | A | 3.083181798 |
| 1KG | 5:113645446 | chr5: 113645446 | 0.0556 | 0.0006648 | G | 3.403698868 |
| 1KG | 5:149749881 | chr5: 149749881 | 0.0698 | 0.006721 | C | 2.710361063 |
| 1KG | 5:165133520 | chr5: 165133520 | 0.0486 | 0.00183 | C | −3.116518819 |
| 1KG | 5:21309600 | rs2883171 | 0.0643 | 0.00001405 | G | 4.343078347 |
| 1KG | 5:26212150 | chr5: 26212150 | 0.0804 | 0.00001452 | C | −4.335846876 |
| 1KG | 5:52621268 | rs251525 | 0.041 | 0.0008037 | G | 3.351517359 |
| 1KG | 5:5291813 | rs7720497 | 0.0816 | 0.0001015 | G | 3.886977878 |
| 1KG | 5:61112540 | rs9291737 | 0.0376 | 0.00009344 | T | −3.907023678 |
| 1KG | 5:64292165 | rs10067691 | 0.1288 | 0.004567 | A | −2.836087023 |
| 1KG | 5:74385314 | rs12520598 | 0.1038 | 0.03102 | T | −2.156816051 |
| 1KG | 5:86468373 | rs2032794 | 0.0458 | 0.2518 | C | 1.145988284 |
| 1KG | 6:109470664 | rs13192030 | 0.6009 | 0.5968 | T | −0.529007856 |
| 1KG | 6:116493827 | rs1999930 | 0.042 | 3.133E−07 | T | −5.115265474 |
| 1KG | 6:116529937 | rs509859 | 0.0388 | 0.00003054 | T | −4.169401925 |
| 1KG | 6:116568331 | rs12204816 | 0.0553 | 1.731E−07 | T | −5.226124315 |
| 1KG | 6:116596243 | rs12196141 | 0.0431 | 1.532E−07 | G | −5.248671475 |
| 1KG | 6:1324870 | rs9328048 | 0.0454 | 0.0002311 | T | −3.68234321 |
| 1KG | 6:132523980 | rs728371 | 0.0414 | 0.00005385 | A | 4.038256293 |
| 1KG | 6:160376153 | rs1867348 | 0.0647 | 0.0005041 | T | 3.478568278 |
| 1KG | 6:162467903 | chr6: 162467903 | 0.0714 | 0.001774 | A | −3.12567083 |
| 1KG | 6:31277268 | rs9366769 | 0.0459 | 0.000005154 | C | −4.558419552 |
| 1KG | 6:32011783 | rs9332739 | 0.1165 | 4.335E−12 | C | −6.925806065 |
| 1KG | 6:32022159 | rs641153 | 0.0798 | 2.94E−22 | A | −9.702569373 |
| 1KG | 6:33503177 | rs9461856 | 0.037 | 0.00007769 | A | −3.951416437 |
| 1KG | 6:43936560 | rs4711751 | 0.0445 | 0.0000218 | T | 4.245609514 |
| 1KG | 6:55456674 | rs4260755 | 0.0387 | 0.000255 | A | 3.657185342 |
| 1KG | 6:57396889 | rs9475939 | 10.7214 | 0.9806 | C | 0.02431669 |
| 1KG | 6:7063989 | rs11755724 | 0.0382 | 0.0006616 | A | −3.405016732 |
| 1KG | 6:7152544 | rs1360751 | 0.0379 | 0.000209 | A | 3.707881999 |
| 1KG | 6:93573222 | rs2037156 | 0.0492 | 0.001602 | G | 3.155542363 |
| HM2 | 7:14123511 | rs7783337 | 0.0552 | 0.002751 | G | 2.99427264 |
| 1KG | 7:23550807 | rs10280782 | 0.0551 | 0.000001593 | G | −4.799200851 |
| 1KG | 7:27475356 | rs17155281 | 0.0513 | 0.01156 | C | −2.525298662 |
| 1KG | 7:35595154 | rs343718 | 0.0406 | 0.1015 | A | −1.637624735 |
| 1KG | 7:43880667 | rs2730613 | 0.0394 | 0.001495 | C | −3.175652343 |
| 1KG | 7:54736904 | rs10225927 | 0.0459 | 0.0002957 | G | 3.619038192 |
| 1KG | 7:86076557 | rs1358395 | 0.042 | 0.0001186 | A | −3.849002081 |
| 1KG | 8:123521501 | rs4282579 | 0.039 | 0.00007708 | G | 3.953302156 |
| 1KG | 8:128550166 | rs10089310 | 0.0599 | 0.0004286 | A | 3.521816937 |
| 1KG | 8:136110326 | rs13253938 | 0.0394 | 0.00001831 | G | −4.284561447 |
| 1KG | 8:13715406 | rs1384044 | 0.038 | 0.0006033 | T | −3.430126462 |
| 1KG | 8:139212254 | rs10103808 | 0.0627 | 0.000006651 | T | 4.504561932 |
| 1KG | 8:14247492 | rs10503493 | 0.0892 | 0.003472 | A | −2.922530154 |
| 1KG | 8:19888502 | rs12678919 | 0.0629 | 0.6846 | G | −0.406194085 |
| 1KG | 8:20081107 | rs2270637 | 0.0482 | 0.0000319 | G | −4.159460629 |
| 1KG | 8:23138916 | rs13278062 | 0.0486 | 0.0006516 | T | 3.409173601 |
| 1KG | 8:506479 | rs722782 | 0.0642 | 0.00004568 | A | −4.076696774 |
| HM2 | 8:96819457 | rs6982567 | 0.051 | 0.0006232 | T | 3.421311102 |
| 1KG | 9:106704122 | rs1883025 | 0.0434 | 0.001275 | T | −3.221548233 |
| 1KG | 9:114121319 | rs10739343 | 0.0454 | 0.0003545 | A | −3.571826678 |
| 1KG | 9:15279578 | rs471364 | 0.0578 | 0.6198 | C | −0.496133819 |
| 1KG | 9:4536594 | rs10815017 | 0.0463 | 0.1325 | T | −1.504316083 |

| REF | hg_18_BP | OldSNP | EA_OR | ci− | ci+ |
|---|---|---|---|---|---|
| 1KG | 1:100166675 | chr1: 100166675 | 1.3684 | 1.144186809 | 1.636549684 |
| 1KG | 1:163986967 | chr1: 163986967 | 0.7049 | 0.550540184 | 0.902539042 |
| 1KG | 1:165795961 | rs1737478 | 1.1357 | 1.042474486 | 1.237262405 |
| 1KG | 1:185513773 | rs2453727 | 1.0017 | 0.931445644 | 1.077253295 |
| 1KG | 1:194925860 | rs1061170 | 2.738975623 | 0.337371025 | 0.395108057 |
| 1KG | 1:194963556 | rs1410996 | 3.118178983 | 0.292993407 | 0.351026636 |
| 1KG | 1:205461564 | rs1367068 | 0.713368526 | 1.232185134 | 1.594762983 |
| 1KG | 1:205515927 | rs12040406 | 0.456183568 | 1.624779296 | 2.957510859 |
| 1KG | 1:227122574 | rs6685751 | 1.1607 | 1.050904552 | 1.281966557 |
| 1KG | 1:228362314 | rs4846914 | 1.040691019 | 0.8919324 | 1.035200436 |
| 1KG | 1:247130153 | rs6587759 | 1.3373 | 1.139421475 | 1.569543254 |
| 1KG | 1:31101812 | rs2491146 | 0.8641 | 0.797534029 | 0.936221883 |
| 1KG | 1:5303445 | rs1326005 | 0.9191 | 0.84912884 | 0.994837026 |
| 1KG | 1:57219300 | rs1418473 | 1.221 | 1.06154585 | 1.404405659 |
| 1KG | 1:57677552 | rs11207037 | 1.0234367 | 0.897069196 | 1.064270643 |
| 1KG | 1:79360392 | rs17408651 | 0.8501 | 0.746068926 | 0.968637059 |
| 1KG | 10:124204438 | rs10490924 | 3.1865 | 2.915773499 | 3.482363172 |
| 1KG | 10:19392088 | rs11596472 | 1.150747986 | 0.708472416 | 1.065900356 |
| 1KG | 10:24883792 | rs16924889 | 0.9404 | 0.823218219 | 1.074262133 |
| 1KG | 10:32036318 | rs2277252 | 1.1865 | 1.089104498 | 1.292605304 |
| 1KG | 10:3945290 | rs11592003 | 0.819470622 | 1.099250661 | 1.354679277 |
| 1KG | 10:56380194 | rs61856267 | 1.354829969 | 0.637822044 | 0.854143589 |
| 1KG | 10:57479107 | chr10: 57479107 | 1.1634 | 1.035742699 | 1.306791312 |
| 1KG | 10:8226099 | rs2798832 | 1.166 | 1.0688119842 | 1.272016057 |
| 1KG | 10:88344492 | chr10: 88344492 | 1.2144 | 1.093721529 | 1.348393829 |
| 1KG | 10:88396729 | rs2803544 | 1.2022 | 1.08230953 | 1.335371074 |
| 1KG | 10:92382787 | rs7094579 | 0.753 | 0.636569261 | 0.890726327 |
| 1KG | 11:100149779 | rs625761 | 0.863185153 | 1.061944928 | 1.263834135 |
| 1KG | 11:103362906 | rs12798294 | 0.8706 | 0.801645611 | 0.945485573 |
| 1KG | 11:104185708 | chr11: 104185708 | 1.7253 | 1.120757671 | 2.655935505 |
| 1KG | 11:116154127 | rs964184 | 0.963483958 | 0.934028284 | 1.153323116 |
| 1KG | 11:1762440 | rs55911157 | 0.7781 | 0.696259928 | 0.869559751 |
| 1KG | 11:1768072 | rs72850977 | 0.7792 | 0.697927865 | 0.869936093 |
| 1KG | 11:32859894 | rs4755455 | 0.9526 | 0.864012372 | 1.050270562 |
| 1KG | 11:37597461 | rs10768315 | 0.8472 | 0.780710806 | 0.919351743 |
| 1KG | 11:61327359 | rs174547 | 0.952380952 | 0.972728922 | 1.133409293 |
| 1KG | 11:73662041 | rs513683 | 0.8991 | 0.833587156 | 0.969761595 |
| 1KG | 12:108379551 | rs2338104 | 0.956022945 | 0.97244804 | 1.125115127 |
| 1KG | 12:11233234 | chr12: 11233234 | 1.1565 | 1.005075037 | 1.330738701 |
| 1KG | 12:114010367 | rs11067403 | 0.871231922 | 1.06208193 | 1.240436169 |
| 1KG | 12:123850701 | rs5888 | 1.002004008 | 0.925099479 | 1.076645294 |
| 1KG | 12:19878442 | rs1492116 | 0.842034355 | 1.087553267 | 1.296850281 |
| 1KG | 12:2290505 | rs10848645 | 1.190617931 | 0.771257915 | 0.914651243 |
| 1KG | 12:45893566 | rs11183802 | 1.3253 | 1.109670194 | 1.582830736 |
| 1KG | 12:50881293 | rs7958529 | 0.844951415 | 1.08571217 | 1.290095375 |
| 1KG | 12:80847264 | rs7973032 | 0.958864704 | 0.900329289 | 1.208047348 |
| 1KG | 12:92165329 | rs7973431 | 1.0087 | 0.930447499 | 1.093533693 |
| 1KG | 13:105365389 | rs4771513 | 0.949667616 | 0.937272922 | 1.183016146 |
| 1KG | 13:30736688 | rs1912795 | 1.1392 | 1.057642381 | 1.227046745 |
| 1KG | 13:78960018 | rs17071512 | 0.805347507 | 1.077006473 | 1.431578109 |
| 1KG | 13:83069357 | rs7317763 | 1.0722 | 0.99563417 | 1.154653862 |
| 1KG | 14:95216646 | rs8011890 | 1.4427 | 1.213902478 | 1.714621502 |
| 1KG | 15:30811243 | rs3743105 | 0.9607 | 0.89157199 | 1.035187849 |
| 1KG | 15:35201758 | chr15: 35201758 | 1.3375 | 1.168314603 | 1.531185389 |
| 1KG | 15:35992085 | rs16965939 | 0.6687 | 0.556836427 | 0.803035987 |
| 1KG | 15:40375218 | rs12908430 | 0.6083 | 0.461058153 | 0.802564464 |
| 1KG | 15:47957127 | chr15: 47957127 | 0.1281 | 0.019366935 | 0.847300327 |
| 1KG | 15:56465804 | rs10468017 | 0.8324 | 0.762126913 | 0.909152725 |
| 1KG | 15:56475172 | rs493258 | 1.179106237 | 0.787845932 | 0.912962269 |
| 1KG | 15:59828725 | rs11854497 | 0.825 | 0.730884457 | 0.931234743 |
| 1KG | 15:87312556 | rs11854658 | 0.859623485 | 1.077479798 | 1.255955696 |
| 1KG | 15:87314645 | rs11856826 | 0.853096741 | 1.082535879 | 1.269290807 |
| 1KG | 16:11814579 | rs2024393 | 1.0619 | 0.932132559 | 1.2097331 |
| 1KG | 16:17310751 | chr16: 17310751 | 0.763067531 | 1.143608648 | 1.501746469 |
| 1KG | 16:23091095 | rs4967980 | 1.204093919 | 0.760387315 | 0.907077533 |
| 1KG | 16:55550825 | rs3764261 | 1.1631 | 1.077083424 | 1.255985916 |
| 1KG | 16:66459571 | rs2271293 | 1.0897 | 0.973749134 | 1.219457916 |
| 1KG | 16:72289810 | rs2127740 | 1.2894 | 1.153103489 | 1.44180672 |
| 1KG | 16:74921678 | rs8053796 | 1.1928 | 1.085698419 | 1.310466898 |
| 1KG | 16:74929641 | rs6564324 | 1.197031362 | 0.760240364 | 0.917990142 |
| 1KG | 16:84929276 | rs12926103 | 1.4308 | 1.207907747 | 1.694822013 |
| 1KG | 16:85846518 | rs3748391 | 0.8754 | 0.814163271 | 0.941242607 |
| 1KG | 17:45980827 | rs8076470 | 1.1472 | 1.062359301 | 1.238816131 |
| 1KG | 17:72483712 | chr17: 72483712 | 0.769882208 | 0.971080172 | 1.737386118 |
| 1KG | 17:73838135 | rs16971055 | 1.3213 | 1.08655061 | 1.606767024 |
| 1KG | 17:77136846 | rs4073997 | 1.1002 | 0.784117797 | 1.54369668 |
| 1KG | 18:34111545 | rs8091635 | 1.1987 | 1.108094237 | 1.296714342 |
| 1KG | 18:34125717 | rs17628762 | 1.188 | 1.101004804 | 1.281869066 |
| 1KG | 18:36481096 | rs2469875 | 0.995520159 | 0.934232358 | 1.080052775 |
| 1KG | 18:45421212 | rs4939883 | 1.0146 | 0.922413611 | 1.115999534 |
| 1KG | 18:53934507 | rs529478 | 0.832986256 | 1.099366535 | 1.310936984 |
| 1KG | 18:68641554 | rs11876415 | 0.940203084 | 0.966961479 | 1.169896614 |
| 1KG | 18:72988032 | rs1789110 | 1.201778632 | 0.77026039 | 0.898904344 |
| 1KG | 19:36639376 | rs2052572 | 1.1729 | 1.082545611 | 1.270795795 |
| 1KG | 19:5792356 | rs3760775 | 0.7243 | 0.611227769 | 0.858289685 |
| HM2 | 19:59425996 | rs7260457 | 0.7477 | 0.61125476 | 0.914602759 |
| 1KG | 19:6669387 | rs2230199 | 1.6842 | 1.49969038 | 1.891410172 |
| 1KG | 19:8375738 | rs2967605 | 1.0509 | 0.951864049 | 1.160240069 |
| 1KG | 2:124751486 | rs9308649 | 0.834724541 | 1.091286745 | 1.315148385 |
| 1KG | 2:180796214 | chr2: 180796214 | 0.8419 | 0.762859019 | 0.929130537 |
| 1KG | 2:189854831 | rs10207860 | 0.9 | 0.774683658 | 1.045588081 |
| 1KG | 2:20201501 | rs6531212 | 0.8491 | 0.786305163 | 0.916909673 |
| 1KG | 2:44132778 | chr2: 44132778 | 0.684415851 | 1.192363625 | 1.790404508 |
| 1KG | 2:71597893 | rs13402171 | 1.0004 | 0.882461533 | 1.134100607 |
| 1KG | 2:76232489 | rs1851808 | 1.149954002 | 0.805604829 | 0.938678782 |
| 1KG | 20:42475778 | rs1800961 | 0.8286 | 0.665286337 | 1.032003698 |
| 1KG | 20:44009909 | rs7679 | 0.943307235 | 0.964346358 | 1.165361388 |
| 1KG | 20:4486243 | rs958646 | 0.9867 | 0.865104269 | 1.12538676 |
| 1KG | 20:56979160 | rs235195 | 1.364 | 1.112468655 | 1.672403075 |
| HM2 | 22:31414511 | rs9621532 | 0.719838756 | 1.15907705 | 1.665011519 |
| 1KG | 22:36259705 | rs1079982 | 0.7054 | 0.588664857 | 0.845284298 |
| 1KG | 22:43172593 | rs135912 | 0.951746455 | 0.954110877 | 1.157067294 |
| 1KG | 22:47467676 | rs5771717 | 1.1586 | 0.97199597 | 1.381028318 |
| 1KG | 3:100878962 | rs13095226 | 1.224439819 | 0.729083182 | 0.914846078 |
| 1KG | 3:101053451 | rs7626245 | 1.3399 | 1.165375071 | 1.540561537 |
| 1KG | 3:118210505 | rs1540819 | 1.1303 | 1.049379549 | 1.217460442 |
| 1KG | 3:119674993 | rs12637095 | 0.836470096 | 1.092001781 | 1.308807619 |
| 1KG | 3:119681130 | rs1915098 | 0.7983 | 0.719111532 | 0.886208692 |
| 1KG | 3:120558458 | rs9713311 | 1.134558657 | 0.818940599 | 0.948623088 |
| 1KG | 3:130591125 | rs12632105 | 0.9737 | 0.873852195 | 1.084956581 |
| 1KG | 3:154434339 | rs16822447 | 0.848896435 | 1.057616811 | 1.312085801 |
| 1KG | 3:156261403 | rs13094238 | 0.745378652 | 1.084803573 | 1.659185686 |
| 1KG | 3:163192835 | rs4256145 | 0.843881857 | 1.0956445 | 1.281642905 |
| 1KG | 3:37504640 | rs11716363 | 1.0861 | 0.982975875 | 1.200042891 |
| 1KG | 3:53233464 | rs12632671 | 1.4031 | 1.170674495 | 1.681671223 |
| 1KG | 3:71584585 | rs2135548 | 1.173984503 | 0.784180919 | 0.925249802 |
| 1KG | 4:110878516 | rs10033900 | 1.3144 | 1.213145154 | 1.424106056 |
| 1KG | 4:128495772 | rs1443179 | 1.7594 | 1.239272906 | 2.497826221 |
| 1KG | 4:162971300 | rs17639345 | 1.0405 | 0.940598755 | 1.151011783 |
| 1KG | 4:165142286 | rs11736266 | 1.051524711 | 0.880669191 | 1.026947473 |
| 1KG | 4:182181154 | rs9997006 | 1.2106 | 1.082420898 | 1.353957932 |
| 1KG | 4:23188024 | chr4: 23188024 | 0.491 | 0.310321274 | 0.776875517 |
| 1KG | 4:36840346 | rs2376241 | 1.148 | 1.056246196 | 1.247724257 |
| 1KG | 4:86877248 | rs28621471 | 0.8532 | 0.777352555 | 0.936447993 |
| 1KG | 5:106936632 | rs164700 | 1.2085 | 1.071474697 | 1.363048753 |
| 1KG | 5:113645446 | chr5: 113645446 | 1.208459215 | 0.74206223 | 0.922774698 |
| 1KG | 5:149749881 | chr5: 149749881 | 1.2083 | 1.053803935 | 1.385446421 |
| 1KG | 5:165133520 | chr5: 165133520 | 0.859328005 | 1.05796641 | 1.280000648 |
| 1KG | 5:21309600 | rs2883171 | 1.322401481 | 0.66665848 | 0.857768193 |
| 1KG | 5:26212150 | chr5: 26212150 | 0.705566923 | 1.210664828 | 1.659203476 |
| 1KG | 5:52621268 | rs251525 | 1.147183664 | 0.804390886 | 0.944641347 |
| 1KG | 5:5291813 | rs7720497 | 1.373249107 | 0.620570822 | 0.854495927 |
| 1KG | 5:61112540 | rs9291737 | 0.863483292 | 1.075821689 | 1.246670916 |
| 1KG | 5:64292165 | rs10067691 | 0.694 | 0.539166248 | 0.893297758 |
| 1KG | 5:74385314 | rs12520598 | 0.7993 | 0.652158966 | 0.979639204 |
| 1KG | 5:86468373 | rs2032794 | 1.053962901 | 0.867339108 | 1.037911737 |
| 1KG | 6:109470664 | rs13192030 | 0.727696114 | 0.423207678 | 4.462172449 |
| 1KG | 6:116493827 | rs1999930 | 0.8068 | 0.743044404 | 0.876026031 |
| 1KG | 6:116529937 | rs509859 | 0.8508 | 0.78849738 | 0.918025422 |
| 1KG | 6:116568331 | rs12204816 | 0.7488 | 0.671882803 | 0.834522684 |
| 1KG | 6:116596243 | rs12196141 | 0.797448166 | 1.152418114 | 1.364535997 |
| 1KG | 6:1324870 | rs9328048 | 0.846 | 0.773971739 | 0.924731439 |
| 1KG | 6:132523980 | rs728371 | 1.1821 | 1.089968196 | 1.282019434 |
| 1KG | 6:160376153 | rs1867348 | 1.2525 | 1.103326331 | 1.421842483 |
| 1KG | 6:162467903 | chr6: 162467903 | 0.8001 | 0.695612477 | 0.920282529 |
| 1KG | 6:31277268 | rs9366769 | 0.81109579 | 1.126826323 | 1.348958911 |
| 1KG | 6:32011783 | rs9332739 | 0.4463 | 0.355189472 | 0.560781514 |
| 1KG | 6:32022159 | rs641153 | 0.461 | 0.394251899 | 0.539048767 |
| 1KG | 6:33503177 | rs9461856 | 0.8641 | 0.803653738 | 0.929092685 |
| 1KG | 6:43936560 | rs4711751 | 1.2081 | 1.10719399 | 1.318202251 |
| 1KG | 6:55456674 | rs4260755 | 1.152 | 1.0678503 | 1.24278094 |
| 1KG | 6:57396889 | rs9475939 | 1.297521733 | 5.76296E−10 | 1030683399 |
| 1KG | 6:7063989 | rs11755724 | 0.878 | 0.814663051 | 0.946261155 |
| 1KG | 6:7152544 | rs1360751 | 1.1507 | 1.068319072 | 1.23943354 |
| 1KG | 6:93573222 | rs2037156 | 1.167951413 | 0.777490942 | 0.942877146 |
| HM2 | 7:14123511 | rs7783337 | 1.179941003 | 0.76059334 | 0.944336759 |
| 1KG | 7:23550807 | rs10280782 | 0.767754319 | 1.169164621 | 1.451041384 |
| 1KG | 7:27475356 | rs17155281 | 0.878580214 | 1.029321726 | 1.25859506 |
| 1KG | 7:35595154 | rs343718 | 0.9357 | 0.864126277 | 1.01320202 |
| 1KG | 7:43880667 | rs2730613 | 0.882378893 | 1.049075952 | 1.224285895 |
| 1KG | 7:54736904 | rs10225927 | 1.180637544 | 0.774127582 | 0.926732255 |
| 1KG | 7:86076557 | rs1358395 | 0.8507 | 0.783475303 | 0.923692792 |
| 1KG | 8:123521501 | rs4282579 | 1.166725003 | 0.794024723 | 0.925185815 |
| 1KG | 8:128550166 | rs10089310 | 1.2349 | 1.098105031 | 1.38873602 |
| 1KG | 8:136110326 | rs13253938 | 0.844808651 | 1.095730349 | 1.27873221 |
| 1KG | 8:13715406 | rs1384044 | 0.8777 | 0.814703994 | 0.945567096 |
| 1KG | 8:139212254 | rs10103808 | 1.326259947 | 0.666806813 | 0.852594768 |
| 1KG | 8:14247492 | rs10503493 | 0.7705 | 0.646910306 | 0.917701023 |
| 1KG | 8:19888502 | rs12678919 | 0.974753875 | 0.906908493 | 1.160503864 |
| 1KG | 8:20081107 | rs2270637 | 0.818330606 | 1.111840623 | 1.343073791 |
| 1KG | 8:23138916 | rs13278062 | 1.1804 | 1.073149051 | 1.298369653 |
| 1KG | 8:506479 | rs722782 | 0.7698 | 0.678781135 | 0.873023733 |
| HM2 | 8:96819457 | rs6982567 | 1.1905 | 1.077252035 | 1.315653351 |
| 1KG | 9:106704122 | rs1883025 | 0.8697 | 0.79877901 | 0.946917834 |
| 1KG | 9:114121319 | rs10739343 | 0.8504 | 0.777997124 | 0.929540917 |
| 1KG | 9:15279578 | rs471364 | 0.971722865 | 0.918876671 | 1.152545107 |
| 1KG | 9:4536594 | rs10815017 | 0.9327 | 0.851786242 | 1.021300001 |

| REF | hg_18_BP | OldSNP | SNP | F_EA_A |
|---|---|---|---|---|
| 1KG | 1:100166675 | chr1: 100166675 | rs115952936 | 0.0493 |
| 1KG | 1:163986967 | chr1: 163986967 | chr1: 163986967 | 0.052 |
| 1KG | 1:165795961 | rs1737478 | rs1737478 | 0.2451 |
| 1KG | 1:185513773 | rs2453727 | rs2453727 | 0.4977 |
| 1KG | 1:194925860 | rs1061170 | rs1061170 | 0.6091 |
| 1KG | 1:194963556 | rs1410996 | rs1410996 | 0.7996 |
| 1KG | 1:205461564 | rs1367068 | rs1367068 | 0.1988 |
| 1KG | 1:205515927 | rs12040406 | rs12040406 | 0.0826 |
| 1KG | 1:227122574 | rs6685751 | rs6685751 | 0.4818 |
| 1KG | 1:228362314 | rs4846914 | rs4846914 | 0.4062 |
| 1KG | 1:247130153 | rs6587759 | rs6587759 | 0.0635 |
| 1KG | 1:31101812 | rs2491146 | rs2491146 | 0.3668 |
| 1KG | 1:5303445 | rs1326005 | rs1326005 | 0.3841 |
| 1KG | 1:57219300 | rs1418473 | rs1418473 | 0.0866 |
| 1KG | 1:57677552 | rs11207037 | rs11207037 | 0.2493 |
| 1KG | 1:79360392 | rs17408651 | rs17408651 | 0.1174 |
| 1KG | 10:124204438 | rs10490924 | rs10490924 | 0.4144 |
| 1KG | 10:19392088 | rs11596472 | rs11596472 | 0.0859 |
| 1KG | 10:24883792 | rs16924889 | rs16924889 | 0.0865 |
| 1KG | 10:32036318 | rs2277252 | rs2277252 | 0.4765 |
| 1KG | 10:3945290 | rs11592003 | rs11592003 | 0.1377 |
| 1KG | 10:56380194 | rs61856267 | rs61856267 | 0.1135 |
| 1KG | 10:57479107 | chr10: 57479107 | chr10: 57479107 | 0.1191 |
| 1KG | 10:8226099 | rs2798832 | rs2798832 | 0.266 |
| 1KG | 10:88344492 | chr10: 88344492 | rs55829833 | 0.1597 |
| 1KG | 10:88396729 | rs2803544 | rs2803544 | 0.1528 |
| 1KG | 10:92382787 | rs7094579 | rs7094579 | 0.0451 |
| 1KG | 11:100149779 | rs625761 | rs625761 | 0.2381 |
| 1KG | 11:103362906 | rs12798294 | rs12798294 | 0.2689 |
| 1KG | 11:104185708 | chr11: 104185708 | chr11: 104185708 | 0.0478 |
| 1KG | 11:116154127 | rs964184 | rs964184 | 0.138 |
| 1KG | 11:1762440 | rs55911157 | rs55911157 | 0.1489 |
| 1KG | 11:1768072 | rs72850977 | rs72850977 | 0.148 |
| 1KG | 11:32859894 | rs4755455 | rs4755455 | 0.1684 |
| 1KG | 11:37597461 | rs10768315 | rs10768315 | 0.2642 |
| 1KG | 11:61327359 | rs174547 | rs174547 | 0.334 |
| 1KG | 11:73662041 | rs513683 | rs513683 | 0.4409 |
| 1KG | 12:108379551 | rs2338104 | rs2338104 | 0.5373 |
| 1KG | 12:11233234 | chr12: 11233234 | chr12: 11233234 | 0.1532 |
| 1KG | 12:114010367 | rs11067403 | rs11067403 | 0.3109 |
| 1KG | 12:123850701 | rs5888 | rs5888 | 0.5111 |
| 1KG | 12:19878442 | rs1492116 | rs1492116 | 0.21 |
| 1KG | 12:2290505 | rs10848645 | rs10848645 | 0.429 |
| 1KG | 12:45893566 | rs11183802 | rs11183802 | 0.0774 |
| 1KG | 12:50881293 | rs7958529 | rs7958529 | 0.3262 |
| 1KG | 12:80847264 | rs7973032 | rs7973032 | 0.0709 |
| 1KG | 12:92165329 | rs7973431 | rs7973431 | 0.3164 |
| 1KG | 13:105365389 | rs4771513 | rs4771513 | 0.1262 |
| 1KG | 13:30736688 | rs1912795 | rs1912795 | 0.4579 |
| 1KG | 13:78960018 | rs17071512 | rs17071512 | 0.0961 |
| 1KG | 13:83069357 | rs7317763 | rs7317763 | 0.4613 |
| 1KG | 14:95216646 | rs8011890 | rs8011890 | 0.099 |
| 1KG | 15:30811243 | rs3743105 | rs3743105 | 0.4142 |
| 1KG | 15:35201758 | chr15: 35201758 | chr15: 35201758 | 0.1508 |
| 1KG | 15:35992085 | rs16965939 | rs16965939 | 0.0907 |
| 1KG | 15:40375218 | rs12908430 | rs12908430 | 0.0812 |
| 1KG | 15:47957127 | chr15: 47957127 | chr15: 47957127 | 0.0105 |
| 1KG | 15:56465804 | rs10468017 | rs10468017 | 0.2564 |
| 1KG | 15:56475172 | rs493258 | rs493258 | 0.5682 |
| 1KG | 15:59828725 | rs11854497 | rs11854497 | 0.1036 |
| 1KG | 15:87312556 | rs11854658 | rs11854658 | 0.3236 |
| 1KG | 15:87314645 | rs11856826 | rs11856826 | 0.3423 |
| 1KG | 16:11814579 | rs2024393 | rs2024393 | 0.16 |
| 1KG | 16:17310751 | chr16: 17310751 | rs56005502 | 0.0836 |
| 1KG | 16:23091095 | rs4967980 | rs4967980 | 0.2565 |
| 1KG | 16:55550825 | rs3764261 | rs3764261 | 0.36 |
| 1KG | 16:66459571 | rs2271293 | rs2271293 | 0.1249 |
| 1KG | 16:72289810 | rs2127740 | rs2127740 | 0.1392 |
| 1KG | 16:74921678 | rs8053796 | rs8053796 | 0.2789 |
| 1KG | 16:74929641 | rs6564324 | rs6564324 | 0.2837 |
| 1KG | 16:84929276 | rs12926103 | rs12926103 | 0.0664 |
| 1KG | 16:85846518 | rs3748391 | rs3748391 | 0.518 |
| 1KG | 17:45980827 | rs8076470 | rs8076470 | 0.4062 |
| 1KG | 17:72483712 | chr17: 72483712 | chr17: 72483712 | 0.0546 |
| 1KG | 17:73838135 | rs16971055 | rs16971055 | 0.096 |
| 1KG | 17:77136846 | rs4073997 | rs4073997 | 0.212 |
| 1KG | 18:34111545 | rs8091635 | rs8091635 | 0.3871 |
| 1KG | 18:34125717 | rs17628762 | rs17628762 | 0.3824 |
| 1KG | 18:36481096 | rs2469875 | rs2469875 | 0.4832 |
| 1KG | 18:45421212 | rs4939883 | rs4939883 | 0.1743 |
| 1KG | 18:53934507 | rs529478 | rs529478 | 0.4834 |
| 1KG | 18:68641554 | rs11876415 | rs11876415 | 0.3702 |
| 1KG | 18:72988032 | rs1789110 | rs1789110 | 0.4122 |
| 1KG | 19:36639376 | rs2052572 | rs2052572 | 0.3482 |
| 1KG | 19:5792356 | rs3760775 | rs3760775 | 0.055 |
| HM2 | 19:59425996 | rs7260457 | rs7260457 | 0.3679 |
| 1KG | 19:6669387 | rs2230199 | rs2230199 | 0.2439 |
| 1KG | 19:8375738 | rs2967605 | rs2967605 | 0.1811 |
| 1KG | 2:124751486 | rs9308649 | rs9308649 | 0.1847 |
| 1KG | 2:180796214 | chr2: 180796214 | chr2: 180796214 | 0.1728 |
| 1KG | 2:189854831 | rs10207860 | rs10207860 | 0.0682 |
| 1KG | 2:20201501 | rs6531212 | rs6531212 | 0.3177 |
| 1KG | 2:44132778 | chr2: 44132778 | chr2: 44132778 | 0.0626 |
| 1KG | 2:71597893 | rs13402171 | rs13402171 | 0.0904 |
| 1KG | 2:76232489 | rs1851808 | rs1851808 | 0.3926 |
| 1KG | 20:42475778 | rs1800961 | rs1800961 | 0.0264 |
| 1KG | 20:44009909 | rs7679 | rs7679 | 0.1804 |
| 1KG | 20:4486243 | rs958646 | rs958646 | 0.0838 |
| 1KG | 20:56979160 | rs235195 | rs235195 | 0.1336 |
| HM2 | 22:31414511 | rs9621532 | rs9621532 | 0.0366 |
| 1KG | 22:36259705 | rs1079982 | rs1079982 | 0.0471 |
| 1KG | 22:43172593 | rs135912 | rs135912 | 0.2652 |
| 1KG | 22:47467676 | rs5771717 | rs5771717 | 0.1034 |
| 1KG | 3:100878962 | rs13095226 | rs13095226 | 0.1228 |
| 1KG | 3:101053451 | rs7626245 | rs7626245 | 0.0904 |
| 1KG | 3:118210505 | rs1540819 | rs1540819 | 0.4652 |
| 1KG | 3:119674993 | rs12637095 | rs12637095 | 0.1955 |
| 1KG | 3:119681130 | rs1915098 | rs1915098 | 0.1414 |
| 1KG | 3:120558458 | rs9713311 | rs9713311 | 0.4048 |
| 1KG | 3:130591125 | rs12632105 | rs12632105 | 0.1669 |
| 1KG | 3:154434339 | rs16822447 | r816822447 | 0.1363 |
| 1KG | 3:156261403 | rs13094238 | rs13094238 | 0.1396 |
| 1KG | 3:163192835 | rs4256145 | rs4256145 | 0.3055 |
| 1KG | 3:37504640 | rs11716363 | rs11716363 | 0.1892 |
| 1KG | 3:53233464 | rs12632671 | rs12632671 | 0.0676 |
| 1KG | 3:71584585 | rs2135548 | rs2135548 | 0.2698 |
| 1KG | 4:110878516 | rs10033900 | rs10033900 | 0.5205 |
| 1KG | 4:128495772 | rs1443179 | rs1443179 | 0.0388 |
| 1KG | 4:162971300 | rs17639345 | rs17639345 | 0.1539 |
| 1KG | 4:165142286 | rs11736266 | rs11736266 | 0.522 |
| 1KG | 4:182181154 | rs9997006 | rs9997006 | 0.3841 |
| 1KG | 4:23188024 | chr4: 23188024 | chr4: 23188024 | 0.0345 |
| 1KG | 4:36840346 | rs2376241 | rs2376241 | 0.2687 |
| 1KG | 4:86877248 | rs28621471 | rs28621471 | 0.2253 |
| 1KG | 5:106936632 | rs164700 | rs164700 | 0.1655 |
| 1KG | 5:113645446 | chr5: 113645446 | chr5: 113645446 | 0.1512 |
| 1KG | 5:149749881 | chr5: 149749881 | chr5: 149749881 | 0.0812 |
| 1KG | 5:165133520 | chr5: 165133520 | chr5: 165133520 | 0.1731 |
| 1KG | 5:21309600 | rs2883171 | rs2883171 | 0.137 |
| 1KG | 5:26212150 | chr5: 26212150 | chr5: 26212150 | 0.0567 |
| 1KG | 5:52621268 | rs251525 | rs251525 | 0.3323 |
| 1KG | 5:5291813 | rs7720497 | rs7720497 | 0.068 |
| 1KG | 5:61112540 | rs9291737 | rs9291737 | 0.3857 |
| 1KG | 5:64292165 | rs10067691 | rs10067691 | 0.0525 |
| 1KG | 5:74385314 | rs12520598 | rs12520598 | 0.0438 |
| 1KG | 5:86468373 | rs2032794 | rs2032794 | 0.2204 |
| 1KG | 6:109470664 | rs13192030 | rs13192030 | 0.0366 |
| 1KG | 6:116493827 | rs1999930 | rs1999930 | 0.2595 |
| 1KG | 6:116529937 | rs509859 | rs509859 | 0.3742 |
| 1KG | 6:116568331 | rs12204816 | rs12204816 | 0.1906 |
| 1KG | 6:116596243 | rs12196141 | rs12196141 | 0.2457 |
| 1KG | 6:1324870 | rs9328048 | rs9328048 | 0.3673 |
| 1KG | 6:132523980 | rs728371 | rs728371 | 0.3437 |
| 1KG | 6:160376153 | rs1867348 | rs1867348 | 0.0986 |
| 1KG | 6:162467903 | chr6: 162467903 | chr6: 162467903 | 0.0781 |
| 1KG | 6:31277268 | rs9366769 | rs9366769 | 0.1984 |
| 1KG | 6:32011783 | rs9332739 | rs9332739 | 0.0231 |
| 1KG | 6:32022159 | rs641153 | rs641153 | 0.0528 |
| 1KG | 6:33503177 | rs9461856 | rs9461856 | 0.4878 |
| 1KG | 6:43936560 | rs4711751 | rs4711751 | 0.5352 |
| 1KG | 6:55456674 | rs4260755 | rs4260755 | 0.3862 |
| 1KG | 6:57396889 | rs9475939 | rs9475939 | 0.0283 |
| 1KG | 6:7063989 | rs11755724 | rs11755724 | 0.3424 |
| 1KG | 6:7152544 | rs1360751 | rs1360751 | 0.5508 |
| 1KG | 6:93573222 | rs2037156 | rs2037156 | 0.2143 |
| HM2 | 7:14123511 | rs7783337 | rs7783337 | 0.1519 |
| 1KG | 7:23550807 | rs10280782 | rs10280782 | 0.1232 |
| 1KG | 7:27475356 | rs17155281 | rs17155281 | 0.276 |
| 1KG | 7:35595154 | rs343718 | rs343718 | 0.2884 |
| 1KG | 7:43880667 | rs2730613 | rs2730613 | 0.3344 |
| 1KG | 7:54736904 | rs10225927 | rs10225927 | 0.2159 |
| 1KG | 7:86076557 | rs1358395 | rs1358395 | 0.2901 |
| 1KG | 8:123521501 | rs4282579 | rs4282579 | 0.3444 |
| 1KG | 8:128550166 | rs10089310 | rs10089310 | 0.1149 |
| 1KG | 8:136110326 | rs13253938 | rs13253938 | 0.5278 |
| 1KG | 8:13715406 | rs1384044 | rs1384044 | 0.5042 |
| 1KG | 8:139212254 | rs10103808 | rs10103808 | 0.1148 |
| 1KG | 8:14247492 | rs10503493 | rs10503493 | 0.0425 |
| 1KG | 8:19888502 | rs12678919 | rs12678919 | 0.0989 |
| 1KG | 8:20081107 | rs2270637 | rs2270637 | 0.1728 |
| 1KG | 8:23138916 | rs13278062 | rs13278062 | 0.5369 |
| 1KG | 8:506479 | rs722782 | rs722782 | 0.0841 |
| HM2 | 8:96819457 | rs6982567 | rs6982567 | 0.2077 |
| 1KG | 9:106704122 | rs1883025 | rs1883025 | 0.2564 |
| 1KG | 9:114121319 | rs10739343 | rs10739343 | 0.2015 |
| 1KG | 9:15279578 | rs471364 | rs471364 | 0.1118 |
| 1KG | 9:4536594 | rs10815017 | rs10815017 | 0.2991 |

| REF | hg_18_BP | OldSNP | F_EA_U | INFO | EA_OR | P |
|---|---|---|---|---|---|---|
| 1KG | 1:100166675 | chr1: 100166675 | 0.0366 | 0.9746 | 1.3684 | 0.0005929 |
| 1KG | 1:163986967 | chr1: 163986967 | 0.059 | 1.0445 | 0.7049 | 0.00555 |
| 1KG | 1:165795961 | rs1737478 | 0.2262 | 1.0175 | 1.1357 | 0.003573 |
| 1KG | 1:185513773 | rs2453727 | 0.492 | 0.9109 | 1.0017 | 0.964 |
| 1KG | 1:194925860 | rs1061170 | 0.3713 | 0.5729 | 2.738975623 | 5.638E−138 |
| 1KG | 1:194963556 | rs1410996 | 0.5794 | 0.8946 | 3.118178983 | 2.147E−134 |
| 1KG | 1:205461564 | rs1367068 | 0.226 | 0.8094 | 0.713368526 | 2.796E−07 |
| 1KG | 1:205515927 | rs12040406 | 0.094 | 0.9597 | 0.456183568 | 2.786E−07 |
| 1KG | 1:227122574 | rs6685751 | 0.4657 | 0.6907 | 1.1607 | 0.003299 |
| 1KG | 1:228362314 | rs4846914 | 0.3957 | 0.1864 | 1.040691019 | 0.2946 |
| 1KG | 1:247130153 | rs6587759 | 0.051 | 0.4828 | 1.3373 | 0.0003756 |
| 1KG | 1:31101812 | rs2491146 | 0.3933 | 0.9737 | 0.8641 | 0.000358 |
| 1KG | 1:5303445 | rs1326005 | 0.4049 | 0.9851 | 0.9191 | 0.03688 |
| 1KG | 1:57219300 | rs1418473 | 0.072 | 0.9102 | 1.221 | 0.005179 |
| 1KG | 1:57677552 | rs11207037 | 0.2431 | 0.9669 | 1.0234367 | 0.5953 |
| 1KG | 1:79360392 | rs17408651 | 0.1288 | 0.9045 | 0.8501 | 0.01478 |
| 1KG | 10:124204438 | rs10490924 | 0.2058 | 0.9371 | 3.1865 | 1.21E−144 |
| 1KG | 10:19392088 | rs11596472 | 0.0826 | 0.9403 | 1.150747986 | 0.1779 |
| 1KG | 10:24883792 | rs16924889 | 0.0931 | 0.8113 | 0.9404 | 0.3655 |
| 1KG | 10:32036318 | rs2277252 | 0.455 | 0.9773 | 1.1865 | 0.00008953 |
| 1KG | 10:3945290 | rs11592003 | 0.1615 | 0.7947 | 0.819470622 | 0.0001869 |
| 1KG | 10:56380194 | rs61856267 | 0.0946 | 0.978 | 1.354829969 | 0.00004618 |
| 1KG | 10:57479107 | chr10: 57479107 | 0.1054 | 0.7311 | 1.1634 | 0.01073 |
| 1KG | 10:8226099 | rs2798832 | 0.2403 | 0.9089 | 1.166 | 0.0005466 |
| 1KG | 10:88344492 | chr10: 88344492 | 0.1406 | 0.4875 | 1.2144 | 0.0002725 |
| 1KG | 10:88396729 | rs2803544 | 0.1335 | 0.8953 | 1.2022 | 0.000592 |
| 1KG | 10:92382787 | rs7094579 | 0.0568 | 0.6766 | 0.753 | 0.0009303 |
| 1KG | 11:100149779 | rs625761 | 0.2625 | 0.9887 | 0.863185153 | 0.0009244 |
| 1KG | 11:103362906 | rs12798294 | 0.2959 | 0.9432 | 0.8706 | 0.0009907 |
| 1KG | 11:104185708 | chr11: 104185708 | 0.0463 | 0.5802 | 1.7253 | 0.01322 |
| 1KG | 11:116154127 | rs964184 | 0.1433 | 1.0036 | 0.963483958 | 0.4885 |
| 1KG | 11:1762440 | rs55911157 | 0.1731 | 0.9543 | 0.7781 | 0.000009621 |
| 1KG | 11:1768072 | rs72850977 | 0.1723 | 0.9861 | 0.7792 | 0.000008946 |
| 1KG | 11:32859894 | rs4755455 | 0.1757 | 0.5199 | 0.9526 | 0.3297 |
| 1KG | 11:37597461 | rs10768315 | 0.2971 | 0.7687 | 0.8472 | 0.00007111 |
| 1KG | 11:61327359 | rs174547 | 0.3448 | 0.9171 | 0.952380952 | 0.2107 |
| 1KG | 11:73662041 | rs513683 | 0.4611 | 0.8833 | 0.8991 | 0.005903 |
| 1KG | 12:108379551 | rs2338104 | 0.5421 | 0.9954 | 0.956022945 | 0.2264 |
| 1KG | 12:11233234 | chr12: 11233234 | 0.1392 | 0.871 | 1.1565 | 0.04241 |
| 1KG | 12:114010367 | rs11067403 | 0.342 | 0.6618 | 0.871231922 | 0.0005053 |
| 1KG | 12:123850701 | rs5888 | 0.5141 | 0.6557 | 1.002004008 | 0.9593 |
| 1KG | 12:19878442 | rs1492116 | 0.2335 | 0.8937 | 0.842034355 | 0.00013 |
| 1KG | 12:2290505 | rs10848645 | 0.3969 | 0.739 | 1.190617931 | 0.00006143 |
| 1KG | 12:45893566 | rs11183802 | 0.0708 | 0.9758 | 1.3253 | 0.001883 |
| 1KG | 12:50881293 | rs7958529 | 0.3494 | 1.0147 | 0.844951415 | 0.0001293 |
| 1KG | 12:80847264 | rs7973032 | 0.0685 | 0.996 | 0.958864704 | 0.5758 |
| 1KG | 12:92165329 | rs7973431 | 0.3168 | 0.7038 | 1.0087 | 0.8331 |
| 1KG | 13:105365389 | rs4771513 | 0.1275 | 0.9026 | 0.949667616 | 0.3845 |
| 1KG | 13:30736688 | rs1912795 | 0.4329 | 1.0068 | 1.1392 | 0.0005884 |
| 1KG | 13:78960018 | rs17071512 | 0.1033 | 0.9223 | 0.805347507 | 0.002856 |
| 1KG | 13:83069357 | rs7317763 | 0.4456 | 0.8723 | 1.0722 | 0.06489 |
| 1KG | 14:95216646 | rs8011890 | 0.0828 | 0.8743 | 1.4427 | 0.00003215 |
| 1KG | 15:30811243 | rs3743105 | 0.4211 | 0.9872 | 0.9607 | 0.293 |
| 1KG | 15:35201758 | chr15: 35201758 | 0.1344 | 1.0006 | 1.3375 | 0.00002486 |
| 1KG | 15:35992085 | rs16965939 | 0.1081 | 0.9764 | 0.6687 | 0.00001622 |
| 1KG | 15:40375218 | rs12908430 | 0.0899 | 0.9176 | 0.6083 | 0.0004389 |
| 1KG | 15:47957127 | chr15: 47957127 | 0.0115 | 0.7763 | 0.1281 | 0.03299 |
| 1KG | 15:56465804 | rs10468017 | 0.2874 | 0.9976 | 0.8324 | 0.00004595 |
| 1KG | 15:56475172 | rs493258 | 0.5286 | 1.0005 | 1.179106237 | 0.00001198 |
| 1KG | 15:59828725 | rs11854497 | 0.1139 | 0.9579 | 0.825 | 0.001868 |
| 1KG | 15:87312556 | rs11854658 | 0.3539 | 0.8503 | 0.859623485 | 0.0001108 |
| 1KG | 15:87314645 | rs11856826 | 0.3733 | 0.7294 | 0.853096741 | 0.00009204 |
| 1KG | 16:11814579 | rs2024393 | 0.1583 | 0.9604 | 1.0619 | 0.3666 |
| 1KG | 16:17310751 | chr16: 17310751 | 0.1009 | 0.8498 | 0.763067531 | 0.0001012 |
| 1KG | 16:23091095 | rs4967980 | 0.2253 | 0.9635 | 1.204093919 | 0.0000362 |
| 1KG | 16:55550825 | rs3764261 | 0.3277 | 0.6994 | 1.1631 | 0.0001162 |
| 1KG | 16:66459571 | rs2271293 | 0.1157 | 0.7374 | 1.0897 | 0.1345 |
| 1KG | 16:72289810 | rs2127740 | 0.1138 | 0.67 | 1.2894 | 0.000008169 |
| 1KG | 16:74921678 | rs8053796 | 0.2638 | 0.9744 | 1.1928 | 0.0002414 |
| 1KG | 16:74929641 | rs6564324 | 0.2675 | 0.5228 | 1.197031362 | 0.0001849 |
| 1KG | 16:84929276 | rs12926103 | 0.0573 | 0.9309 | 1.4308 | 0.00003384 |
| 1KG | 16:85846518 | rs3748391 | 0.5459 | 0.993 | 0.8754 | 0.0003254 |
| 1KG | 17:45980827 | rs8076470 | 0.3844 | 1.0049 | 1.1472 | 0.0004619 |
| 1KG | 17:72483712 | chr17: 72483712 | 0.0572 | 0.9544 | 0.769882208 | 0.07803 |
| 1KG | 17:73838135 | rs16971055 | 0.0876 | 0.9963 | 1.3213 | 0.00524 |
| 1KG | 17:77136846 | rs4073997 | 0.2118 | 0.8709 | 1.1002 | 0.5804 |
| 1KG | 18:34111545 | rs8091635 | 0.3485 | 1.0123 | 1.1987 | 0.000006243 |
| 1KG | 18:34125717 | rs17628762 | 0.3426 | 0.9375 | 1.188 | 0.000008979 |
| 1KG | 18:36481096 | rs2469875 | 0.4837 | 0.9992 | 0.995520159 | 0.9027 |
| 1KG | 18:45421212 | rs4939883 | 0.1714 | 0.2388 | 1.0146 | 0.7654 |
| 1KG | 18:53934507 | rs529478 | 0.5103 | 0.9232 | 0.832986256 | 0.00004656 |
| 1KG | 18:68641554 | rs11876415 | 0.3703 | 0.9964 | 0.940203084 | 0.205 |
| 1KG | 18:72988032 | rs1789110 | 0.3749 | 0.9875 | 1.201778632 | 0.000003171 |
| 1KG | 19:36639376 | rs2052572 | 0.3141 | 0.9939 | 1.1729 | 0.00009603 |
| 1KG | 19:5792356 | rs3760775 | 0.0679 | 0.9104 | 0.7243 | 0.0001966 |
| HM2 | 19:59425996 | rs7260457 | 0.3736 | 0.966 | 0.7477 | 0.004664 |
| 1KG | 19:6669387 | rs2230199 | 0.1926 | 0.9598 | 1.6842 | 1.357E−18 |
| 1KG | 19:8375738 | rs2967605 | 0.1762 | 0.9849 | 1.0509 | 0.3256 |
| 1KG | 2:124751486 | rs9308649 | 0.2092 | 0.9488 | 0.834724541 | 0.0001473 |
| 1KG | 2:180796214 | chr2: 180796214 | 0.1957 | 0.809 | 0.8419 | 0.0006251 |
| 1KG | 2:189854831 | rs10207860 | 0.0719 | 0.9044 | 0.9 | 0.1687 |
| 1KG | 2:20201501 | rs6531212 | 0.3514 | 0.5525 | 0.8491 | 0.00003021 |
| 1KG | 2:44132778 | chr2: 44132778 | 0.0756 | 1.0232 | 0.684415851 | 0.0002569 |
| 1KG | 2:71597893 | rs13402171 | 0.091 | 0.8994 | 1.0004 | 0.9956 |
| 1KG | 2:76232489 | rs1851808 | 0.3625 | 1.0133 | 1.149954002 | 0.0003417 |
| 1KG | 20:42475778 | rs1800961 | 0.0314 | 0.9294 | 0.8286 | 0.09321 |
| 1KG | 20:44009909 | rs7679 | 0.1865 | 0.438 | 0.943307235 | 0.227 |
| 1KG | 20:4486243 | rs958646 | 0.087 | 0.8446 | 0.9867 | 0.8419 |
| 1KG | 20:56979160 | rs235195 | 0.1251 | 0.9237 | 1.364 | 0.002827 |
| HM2 | 22:31414511 | rs9621532 | 0.0512 | 0.9988 | 0.719838756 | 0.00037321 |
| 1KG | 22:36259705 | rs1079982 | 0.065 | 0.9484 | 0.7054 | 0.0001557 |
| 1KG | 22:43172593 | rs135912 | 0.2735 | 0.9844 | 0.951746455 | 0.3149 |
| 1KG | 22:47467676 | rs5771717 | 0.1002 | 0.9566 | 1.1586 | 0.1004 |
| 1KG | 3:100878962 | rs13095226 | 0.104 | 0.9379 | 1.224439819 | 0.0004761 |
| 1KG | 3:101053451 | rs7626245 | 0.0727 | 0.9804 | 1.3399 | 0.00003955 |
| 1KG | 3:118210505 | rs1540819 | 0.4345 | 0.2953 | 1.1303 | 0.001249 |
| 1KG | 3:119674993 | rs12637095 | 0.2203 | 0.8651 | 0.836470096 | 0.0001092 |
| 1KG | 3:119681130 | rs1915098 | 0.1655 | 0.9078 | 0.7983 | 0.00002407 |
| 1KG | 3:120558458 | rs9713311 | 0.3765 | 0.9709 | 1.134558657 | 0.0007681 |
| 1KG | 3:130591125 | rs12632105 | 0.1695 | 0.94 | 0.9737 | 0.6286 |
| 1KG | 3:154434339 | rs16822447 | 0.1558 | 0.6039 | 0.848896435 | 0.002918 |
| 1KG | 3:156261403 | rs13094238 | 0.1461 | 0.6745 | 0.745378652 | 0.006705 |
| 1KG | 3:163192835 | rs4256145 | 0.3381 | 0.1812 | 0.843881857 | 0.00002249 |
| 1KG | 3:37504640 | rs11716363 | 0.179 | 0.8854 | 1.0861 | 0.105 |
| 1KG | 3:53233464 | rs12632671 | 0.0568 | 0.2803 | 1.4031 | 0.0002466 |
| 1KG | 3:71584585 | rs2135548 | 0.2402 | 0.7645 | 1.173984503 | 0.0001424 |
| 1KG | 4:110878516 | rs10033900 | 0.463 | 0.919 | 1.3144 | 2.401E−11 |
| 1KG | 4:128495772 | rs1443179 | 0.0348 | 0.5355 | 1.7594 | 0.001578 |
| 1KG | 4:162971300 | rs17639345 | 0.1466 | 0.9682 | 1.0405 | 0.4416 |
| 1KG | 4:165142286 | rs11736266 | 0.5173 | 0.9843 | 1.051524711 | 0.1999 |
| 1KG | 4:182181154 | rs9997006 | 0.3654 | 0.3968 | 1.2106 | 0.0008115 |
| 1KG | 4:23188024 | chr4: 23188024 | 0.0389 | 0.1399 | 0.491 | 0.002383 |
| 1KG | 4:36840346 | rs2376241 | 0.2423 | 0.9059 | 1.148 | 0.001166 |
| 1KG | 4:86877248 | rs28621471 | 0.2481 | 0.3957 | 0.8532 | 0.0008447 |
| 1KG | 5:106936632 | rs164700 | 0.142 | 0.3956 | 1.2085 | 0.002048 |
| 1KG | 5:113645446 | chr5: 113645446 | 0.132 | 0.9279 | 1.208459215 | 0.0006648 |
| 1KG | 5:149749881 | chr5: 149749881 | 0.0713 | 0.2446 | 1.2083 | 0.006721 |
| 1KG | 5:165133520 | chr5: 165133520 | 0.1948 | 0.98 | 0.859328005 | 0.00183 |
| 1KG | 5:21309600 | rs2883171 | 0.116 | 0.9596 | 1.322401481 | 0.00001405 |
| 1KG | 5:26212150 | chr5: 26212150 | 0.0733 | 0.6461 | 0.705566923 | 0.00001452 |
| 1KG | 5:52621268 | rs251525 | 0.3101 | 0.1572 | 1.147183664 | 0.0008037 |
| 1KG | 5:5291813 | rs7720497 | 0.0546 | 0.7188 | 1.373249107 | 0.0001015 |
| 1KG | 5:61112540 | rs9291737 | 0.4211 | 0.7485 | 0.863483292 | 0.00009344 |
| 1KG | 5:64292165 | rs10067691 | 0.0594 | 0.0401 | 0.694 | 0.0045671 |
| 1KG | 5:74385314 | rs12520598 | 0.0509 | 0.8639 | 0.7993 | 0.03102 |
| 1KG | 5:86468373 | rs2032794 | 0.2108 | 0.501 | 1.053962901 | 0.2518 |
| 1KG | 6:109470664 | rs13192030 | 0.0357 | 0.9587 | 0.727696114 | 0.5968 |
| 1KG | 6:116493827 | rs1999930 | 0.3044 | 0.2794 | 0.8068 | 3.133E−07 |
| 1KG | 6:116529937 | rs509859 | 0.4133 | 0.9893 | 0.8508 | 0.00003054 |
| 1KG | 6:116568331 | rs12204816 | 0.224 | 0.4444 | 0.7488 | 1.731E−07 |
| 1KG | 6:116596243 | rs12196141 | 0.2872 | 0.9876 | 0.797448166 | 1.532E−07 |
| 1KG | 6:1324870 | rs9328048 | 0.395 | 0.8669 | 0.846 | 0.0002311 |
| 1KG | 6:132523980 | rs728371 | 0.3145 | 0.7359 | 1.1821 | 0.00005385 |
| 1KG | 6:160376153 | rs1867348 | 0.0801 | 0.9686 | 1.2525 | 0.0005041 |
| 1KG | 6:162467903 | chr6: 162467903 | 0.09 | 0.8867 | 0.8001 | 0.001774 |
| 1KG | 6:31277268 | rs9366769 | 0.2305 | 0.4034 | 0.81109579 | 0.000005154 |
| 1KG | 6:32011783 | rs9332739 | 0.0447 | 0.8879 | 0.4463 | 4.335E−12 |
| 1KG | 6:32022159 | rs641153 | 0.101 | 0.6268 | 0.461 | 2.94E−22 |
| 1KG | 6:33503177 | rs9461856 | 0.5204 | 0.9963 | 0.8641 | 0.00007769 |
| 1KG | 6:43936560 | rs4711751 | 0.5044 | 0.9915 | 1.2081 | 0.0000218 |
| 1KG | 6:55456674 | rs4260755 | 0.3512 | 0.9741 | 1.152 | 0.000255 |
| 1KG | 6:57396889 | rs9475939 | 0.0284 | 0.9482 | 1.297521733 | 0.9806 |
| 1KG | 6:7063989 | rs11755724 | 0.3723 | 0.9587 | 0.878 | 0.0006616 |
| 1KG | 6:7152544 | rs1360751 | 0.5208 | 0.9758 | 1.1507 | 0.000209 |
| 1KG | 6:93573222 | rs2037156 | 0.1926 | 0.711 | 1.167951413 | 0.001602 |
| HM2 | 7:14123511 | rs7783337 | 0.1375 | 0.9119 | 1.179941003 | 0.002751 |
| 1KG | 7:23550807 | rs10280782 | 0.1513 | 0.9646 | 0.767754319 | 0.000001593 |
| 1KG | 7:27475356 | rs17155281 | 0.2917 | 0.92 | 0.878580214 | 0.01156 |
| 1KG | 7:35595154 | rs343718 | 0.3029 | 0.5758 | 0.9357 | 0.1015 |
| 1KG | 7:43880667 | rs2730613 | 0.3525 | 0.8741 | 0.882378893 | 0.001495 |
| 1KG | 7:54736904 | rs10225927 | 0.1905 | 1.0039 | 1.180637544 | 0.0002957 |
| 1KG | 7:86076557 | rs1358395 | 0.3185 | 0.9776 | 0.8507 | 0.0001186 |
| 1KG | 8:123521501 | rs4282579 | 0.3112 | 0.9314 | 1.166725003 | 0.00007708 |
| 1KG | 8:128550166 | rs10089310 | 0.0961 | 0.0677 | 1.2349 | 0.0004286 |
| 1KG | 8:136110326 | rs13253938 | 0.5635 | 0.9637 | 0.844808651 | 0.00001831 |
| 1KG | 8:13715406 | rs1384044 | 0.5311 | 0.0274 | 0.8777 | 0.0006033 |
| 1KG | 8:139212254 | rs10103808 | 0.0905 | 1.011 | 1.326259947 | 0.000006651 |
| 1KG | 8:14247492 | rs10503493 | 0.055 | 0.8234 | 0.7705 | 0.003472 |
| 1KG | 8:19888502 | rs12678919 | 0.101 | 0.9593 | 0.974753875 | 0.6846 |
| 1KG | 8:20081107 | rs2270637 | 0.1999 | 1.008 | 0.818330606 | 0.0000319 |
| 1KG | 8:23138916 | rs13278062 | 0.5138 | 0.9277 | 1.1804 | 0.0006516 |
| 1KG | 8:506479 | rs722782 | 0.1055 | 0.9723 | 0.7698 | 0.00004568 |
| HM2 | 8:96819457 | rs6982567 | 0.1912 | 0.9911 | 1.1905 | 0.0006232 |
| 1KG | 9:106704122 | rs1883025 | 0.2784 | 0.9172 | 0.8697 | 0.001275 |
| 1KG | 9:114121319 | rs10739343 | 0.2256 | 0.9901 | 0.8504 | 0.0003545 |
| 1KG | 9:15279578 | rs471364 | 0.1143 | 0.0001 | 0.971722865 | 0.6198 |
| 1KG | 9:4536594 | rs10815017 | 0.3153 | 1.0083 | 0.9327 | 0.1325 |

TABLE 7

| REF | hg_18_BP | OldSNP | A1 | A2 | FRQ_A(2594) | FRQ_U(4134) | OR | SE |
|---|---|---|---|---|---|---|---|---|
| 1KG | 10:124204438 | rs10490924 | T | G | 0.4144 | 0.2058 | 3.1865 | 0.0453 |
| 1KG | 1:194925860 | rs1061170 | T | C | 0.3909 | 0.6287 | 0.3651 | 0.0403 |
| 1KG | 1:194963556 | rs1410996 | A | G | 0.2004 | 0.4206 | 0.3207 | 0.0461 |
| 1KG | 6:32022159 | rs641153 | A | G | 0.0528 | 0.101 | 0.461 | 0.0798 |
| 1KG | 19:6669387 | rs2230199 | C | G | 0.2439 | 0.1926 | 1.6842 | 0.0592 |
| 1KG | 6:32011783 | rs9332739 | C | G | 0.0231 | 0.0447 | 0.4463 | 0.1165 |
| 1KG | 4:110878516 | rs10033900 | T | C | 0.5205 | 0.463 | 1.3144 | 0.0409 |
| 1KG | 6:116596243 | rs12196141 | A | G | 0.7543 | 0.7128 | 1.254 | 0.0431 |
| 1KG | 6:116568331 | rs12204816 | T | C | 0.1906 | 0.224 | 0.7488 | 0.0553 |
| 1KG | 1:205515927 | rs12040406 | T | C | 0.9174 | 0.906 | 2.1921 | 0.1528 |
| 1KG | 18:34125717 | rs17628762 | A | C | 0.3824 | 0.3426 | 1.188 | 0.0388 |
| 1KG | 11:1762440 | rs55911157 | T | C | 0.1489 | 0.1731 | 0.7781 | 0.0567 |
| 1KG | 15:56475172 | rs493258 | T | C | 0.4318 | 0.4714 | 0.8481 | 0.0376 |
| 1KG | 6:116529937 | rs509859 | T | G | 0.3742 | 0.4133 | 0.8508 | 0.0388 |
| 1KG | 15:56465804 | rs10468017 | T | C | 0.2564 | 0.2874 | 0.8324 | 0.045 |
| 1KG | 8:123521501 | rs4282579 | A | G | 0.6556 | 0.6888 | 0.8571 | 0.039 |
| 1KG | 15:87314645 | rs11856826 | T | C | 0.6577 | 0.6267 | 1.1722 | 0.0406 |
| 1KG | 3:119674993 | rs12637095 | A | T | 0.8045 | 0.7797 | 1.1955 | 0.0462 |
| 1KG | 16:55550825 | rs3764261 | A | C | 0.36 | 0.3277 | 1.1631 | 0.0392 |
| 1KG | 16:74929641 | rs6564324 | A | G | 0.7163 | 0.7325 | 0.8354 | 0.0481 |
| 1KG | 16:85846518 | rs3748391 | T | G | 0.518 | 0.5459 | 0.8754 | 0.037 |
| 1KG | 9:114121319 | rs10739343 | A | G | 0.2015 | 0.2256 | 0.8504 | 0.0454 |
| HM2 | 22:31414511 | rs9621532 | A | C | 0.9634 | 0.9488 | 1.3892 | 0.0924 |
| 1KG | 8:128550166 | rs10089310 | A | T | 0.1149 | 0.0961 | 1.2349 | 0.0599 |
| 1KG | 3:100878962 | rs13095226 | T | C | 0.8772 | 0.896 | 0.8167 | 0.0579 |
| 1KG | 10:88396729 | rs2803544 | A | T | 0.1528 | 0.1335 | 1.2022 | 0.0536 |
| 1KG | 6:7063989 | rs11755724 | A | G | 0.3424 | 0.3723 | 0.878 | 0.0382 |
| 1KG | 4:182181154 | rs9997006 | T | G | 0.3841 | 0.3654 | 1.2106 | 0.0571 |
| 1KG | 9:106704122 | rs1883025 | T | C | 0.2564 | 0.2784 | 0.8697 | 0.0434 |
| 1KG | 15:59828725 | rs11854497 | A | G | 0.1036 | 0.1139 | 0.825 | 0.0618 |
| 1KG | 1:5303445 | rs1326005 | A | G | 0.3841 | 0.4049 | 0.9191 | 0.0404 |
| 1KG | 13:83069357 | rs7317763 | T | C | 0.4613 | 0.4456 | 1.0722 | 0.0378 |
| 1KG | 20:42475778 | rs1800961 | T | C | 0.0264 | 0.0314 | 0.8286 | 0.112 |
| 1KG | 3:37504640 | rs11716363 | T | C | 0.1892 | 0.179 | 1.0861 | 0.0509 |
| 1KG | 9:4536594 | rs10815017 | T | C | 0.2991 | 0.3153 | 0.9327 | 0.0463 |
| 1KG | 16:66459571 | rs2271293 | A | G | 0.1249 | 0.1157 | 1.0897 | 0.0574 |
| 1KG | 2:189854831 | rs10207860 | T | C | 0.0682 | 0.0719 | 0.9 | 0.0765 |
| 1KG | 4:165142286 | rs11736266 | T | C | 0.478 | 0.4827 | 0.951 | 0.0392 |
| 1KG | 18:68641554 | rs11876415 | T | C | 0.6298 | 0.6297 | 1.0636 | 0.0486 |
| 1KG | 11:61327359 | rs174547 | T | C | 0.666 | 0.6552 | 1.05 | 0.039 |
| 1KG | 12:108379551 | rs2338104 | C | G | 0.4627 | 0.4579 | 1.046 | 0.0372 |
| 1KG | 20:44009909 | rs7679 | T | C | 0.8196 | 0.8135 | 1.0601 | 0.0483 |
| 1KG | 5:86468373 | rs2032794 | T | C | 0.7796 | 0.7892 | 0.9488 | 0.0458 |
| 1KG | 15:30811243 | rs3743105 | T | C | 0.4142 | 0.4211 | 0.9607 | 0.0381 |
| 1KG | 1:228362314 | rs4846914 | A | G | 0.5938 | 0.6043 | 0.9609 | 0.038 |
| 1KG | 19:8375738 | rs2967605 | T | C | 0.1811 | 0.1762 | 1.0509 | 0.0505 |
| 1KG | 11:32859894 | rs4755455 | C | G | 0.1684 | 0.1757 | 0.9526 | 0.0498 |
| 1KG | 10:24883792 | rs16924889 | A | G | 0.0865 | 0.0931 | 0.9404 | 0.0679 |
| 1KG | 13:105365389 | rs4771513 | T | C | 0.8738 | 0.8725 | 1.053 | 0.0594 |
| 1KG | 4:162971300 | rs17639345 | A | G | 0.1539 | 0.1466 | 1.0405 | 0.0515 |
| 1KG | 11:116154127 | rs964184 | C | G | 0.862 | 0.8567 | 1.0379 | 0.0538 |
| 1KG | 12:80847264 | rs7973032 | A | G | 0.9291 | 0.9315 | 1.0429 | 0.075 |
| 1KG | 1:57677552 | rs11207037 | A | G | 0.7507 | 0.7569 | 0.9771 | 0.0436 |
| 1KG | 9:15279578 | rs471364 | T | C | 0.8882 | 0.8857 | 1.0291 | 0.0578 |
| 1KG | 8:19888502 | rs12678919 | A | G | 0.9011 | 0.899 | 1.0259 | 0.0629 |
| 1KG | 18:45421212 | rs4939883 | T | C | 0.1743 | 0.1714 | 1.0146 | 0.0486 |
| 1KG | 12:92165329 | rs7973431 | T | C | 0.3164 | 0.3168 | 1.0087 | 0.0412 |
| 1KG | 20:4486243 | rs958646 | C | G | 0.0838 | 0.087 | 0.9867 | 0.0671 |
| 1KG | 18:36481096 | rs2469875 | A | G | 0.5168 | 0.5163 | 1.0045 | 0.037 |
| 1KG | 12:123850701 | rs5888 | A | G | 0.4889 | 0.4859 | 0.998 | 0.0387 |
| 1KG | 1:185513773 | rs2453727 | T | C | 0.4977 | 0.492 | 1.0017 | 0.0371 |
| 1KG | 6:57396889 | rs9475939 | T | C | 0.9717 | 0.9716 | 0.7707 | 10.7214 |
| 1KG | 2:71597893 | rs13402171 | T | C | 0.0904 | 0.091 | 1.0004 | 0.064 |

| REF | hg_18_BP | OldSNP | P | EA | Z | EA_OR |
|---|---|---|---|---|---|---|
| 1KG | 10:124204438 | rs10490924 | 1.21E−144 | T | 25.60903658 | 3.1865 |
| 1KG | 1:194925860 | rs1061170 | 5.638E−138 | C | 25.00323274 | 2.738975623 |
| 1KG | 1:194963556 | rs1410996 | 2.147E−134 | G | 24.67181558 | 3.118178983 |
| 1KG | 6:32022159 | rs641153 | 2.94E−22 | A | −9.702569373 | 0.461 |
| 1KG | 19:6669387 | rs2230199 | 1.357E−18 | C | 8.800919225 | 1.6842 |
| 1KG | 6:32011783 | rs9332739 | 4.335E−12 | C | −6.925806065 | 0.4463 |
| 1KG | 4:110878516 | rs10033900 | 2.401E−11 | T | 6.679291601 | 1.3144 |
| 1KG | 6:116596243 | rs12196141 | 1.532E−07 | G | −5.248671475 | 0.797448166 |
| 1KG | 6:116568331 | rs12204816 | 1.731E−07 | T | −5.226124315 | 0.7488 |
| 1KG | 1:205515927 | rs12040406 | 2.786E−07 | C | −5.137376796 | 0.456183568 |
| 1KG | 18:34125717 | rs17628762 | 0.000008979 | A | 4.440404368 | 1.188 |
| 1KG | 11:1762440 | rs55911157 | 0.000009621 | T | −4.425520544 | 0.7781 |
| 1KG | 15:56475172 | rs493258 | 0.00001198 | C | 4.377951501 | 1.179106237 |
| 1KG | 6:116529937 | rs509859 | 0.00003054 | T | −4.169401925 | 0.8508 |
| 1KG | 15:56465804 | rs10468017 | 0.00004595 | T | −4.075325644 | 0.8324 |
| 1KG | 8:123521501 | rs4282579 | 0.00007708 | G | 3.953302156 | 1.166725003 |
| 1KG | 15:87314645 | rs11856826 | 0.00009204 | C | −3.910671187 | 0.853096741 |
| 1KG | 3:119674993 | rs12637095 | 0.0001092 | T | −3.869184471 | 0.836470096 |
| 1KG | 16:55550825 | rs3764261 | 0.0001162 | A | 3.85400757 | 1.1631 |
| 1KG | 16:74929641 | rs6564324 | 0.0001849 | G | 3.738801021 | 1.197031362 |
| 1KG | 16:85846518 | rs3748391 | 0.0003254 | T | −3.594191857 | 0.8754 |
| 1KG | 9:114121319 | rs10739343 | 0.0003545 | A | −3.571826678 | 0.8504 |
| HM2 | 22:31414511 | rs9621532 | 0.0003732 | C | −3.558344238 | 0.719838756 |
| 1KG | 8:128550166 | rs10089310 | 0.0004286 | A | 3.521816937 | 1.2349 |
| 1KG | 3:100878962 | rs13095226 | 0.0004761 | C | 3.493854735 | 1.224439819 |
| 1KG | 10:88396729 | rs2803544 | 0.000592 | A | 3.435253392 | 1.2022 |
| 1KG | 6:7063989 | rs11755724 | 0.0006616 | A | −3.405016732 | 0.878 |
| 1KG | 4:182181154 | rs9997006 | 0.0008115 | T | 3.3488422 | 1.2106 |
| 1KG | 9:106704122 | rs1883025 | 0.001275 | T | −3.221548233 | 0.8697 |
| 1KG | 15:59828725 | rs11854497 | 0.001868 | A | −3.110453982 | 0.825 |
| 1KG | 1:5303445 | rs1326005 | 0.03688 | A | −2.087089998 | 0.9191 |
| 1KG | 13:83069357 | rs7317763 | 0.06489 | T | 1.846015189 | 1.0722 |
| 1KG | 20:42475778 | rs1800961 | 0.09321 | T | −1.678702697 | 0.8286 |
| 1KG | 3:37504640 | rs11716363 | 0.105 | T | 1.621082251 | 1.0861 |
| 1KG | 9:4536594 | rs10815017 | 0.1325 | T | −1.504316083 | 0.9327 |
| 1KG | 16:66459571 | rs2271293 | 0.1345 | A | 1.496589899 | 1.0897 |
| 1KG | 2:189854831 | rs10207860 | 0.1687 | T | −1.376392981 | 0.9 |
| 1KG | 4:165142286 | rs11736266 | 0.1999 | C | 1.281836521 | 1.051524711 |
| 1KG | 18:68641554 | rs11876415 | 0.205 | C | −1.267434417 | 0.940203084 |
| 1KG | 11:61327359 | rs174547 | 0.2107 | C | −1.251642941 | 0.952380952 |
| 1KG | 12:108379551 | rs2338104 | 0.2264 | G | −1.209684447 | 0.956022945 |
| 1KG | 20:44009909 | rs7679 | 0.227 | C | −1.20812288 | 0.943307235 |
| 1KG | 5:86468373 | rs2032794 | 0.2518 | C | 1.145988284 | 1.053962901 |
| 1KG | 15:30811243 | rs3743105 | 0.293 | T | −1.051563198 | 0.9607 |
| 1KG | 1:228362314 | rs4846914 | 0.2946 | G | 1.048083799 | 1.040691019 |
| 1KG | 19:8375738 | rs2967605 | 0.3256 | T | 0.983015114 | 1.0509 |
| 1KG | 11:32859894 | rs4755455 | 0.3297 | C | −0.974718326 | 0.9526 |
| 1KG | 10:24883792 | rs16924889 | 0.3655 | A | −0.904934667 | 0.9404 |
| 1KG | 13:105365389 | rs4771513 | 0.3845 | C | −0.869634825 | 0.949667616 |
| 1KG | 4:162971300 | rs17639345 | 0.4416 | A | 0.769494177 | 1.0405 |
| 1KG | 11:116154127 | rs964184 | 0.4885 | G | −0.692696557 | 0.963483958 |
| 1KG | 12:80847264 | rs7973032 | 0.5758 | G | −0.559530092 | 0.958864704 |
| 1KG | 1:57677552 | rs11207037 | 0.5953 | G | 0.531171414 | 1.0234367 |
| 1KG | 9:15279578 | rs471364 | 0.6198 | C | −0.496133819 | 0.971722865 |
| 1KG | 8:19888502 | rs12678919 | 0.6846 | G | −0.406194085 | 0.974753875 |
| 1KG | 18:45421212 | rs4939883 | 0.7654 | T | 0.298397234 | 1.0146 |
| 1KG | 12:92165329 | rs7973431 | 0.8331 | T | 0.21072739 | 1.0087 |
| 1KG | 20:4486243 | rs958646 | 0.8419 | C | −0.199463746 | 0.9867 |
| 1KG | 18:36481096 | rs2469875 | 0.9027 | G | −0.122251299 | 0.995520159 |
| 1KG | 12:123850701 | rs5888 | 0.9593 | G | 0.051032027 | 1.002004008 |
| 1KG | 1:185513773 | rs2453727 | 0.964 | T | 0.045134628 | 1.0017 |
| 1KG | 6:57396889 | rs9475939 | 0.9806 | C | 0.02431669 | 1.297521733 |
| 1KG | 2:71597893 | rs13402171 | 0.9956 | T | 0.00551461 | 1.0004 |

| REF | hg_18_BP | OldSNP | ci− | ci+ |
|---|---|---|---|---|
| 1KG | 10:124204438 | rs10490924 | 2.915773499 | 3.482363172 |
| 1KG | 1:194925860 | rs1061170 | 0.337371025 | 0.395108057 |
| 1KG | 1:194963556 | rs1410996 | 0.292993407 | 0.351026636 |
| 1KG | 6:32022159 | rs641153 | 0.394251899 | 0.539048767 |
| 1KG | 19:6669387 | rs2230199 | 1.49969038 | 1.891410172 |
| 1KG | 6:32011783 | rs9332739 | 0.355189472 | 0.560781514 |
| 1KG | 4:110878516 | rs10033900 | 1.213145154 | 1.424106056 |
| 1KG | 6:116596243 | rs12196141 | 1.152418114 | 1.364535997 |
| 1KG | 6:116568331 | rs12204816 | 0.671882803 | 0.834522684 |
| 1KG | 1:205515927 | rs12040406 | 1.624779296 | 2.957510859 |
| 1KG | 18:34125717 | rs17628762 | 1.101004804 | 1.281869066 |
| 1KG | 11:1762440 | rs55911157 | 0.696259928 | 0.869559751 |
| 1KG | 15:56475172 | rs493258 | 0.787845932 | 0.912962269 |
| 1KG | 6:116529937 | rs509859 | 0.78849738 | 0.918025422 |
| 1KG | 15:56465804 | rs10468017 | 0.762126913 | 0.9091527251 |
| 1KG | 8:123521501 | rs4282579 | 0.794024723 | 0.925185815 |
| 1KG | 15:87314645 | rs11856826 | 1.082535879 | 1.269290807 |
| 1KG | 3:119674993 | rs12637095 | 1.092001781 | 1.308807619 |
| 1KG | 16:55550825 | rs3764261 | 1.077083424 | 1.255985916 |
| 1KG | 16:74929641 | rs6564324 | 0.760240364 | 0.917990142 |
| 1KG | 16:85846518 | rs3748391 | 0.814163271 | 0.941242607 |
| 1KG | 9:114121319 | rs10739343 | 0.777997124 | 0.929540917 |
| HM2 | 22:31414511 | rs9621532 | 1.15907705 | 1.665011519 |
| 1KG | 8:128550166 | rs10089310 | 1.098105031 | 1.38873602 |
| 1KG | 3:100878962 | rs13095226 | 0.729083182 | 0.914846078 |
| 1KG | 10:88396729 | rs2803544 | 1.08230953 | 1.335371074 |
| 1KG | 6:7063989 | rs11755724 | 0.814663051 | 0.946261155 |
| 1KG | 4:182181154 | rs9997006 | 1.082420898 | 1.353957932 |
| 1KG | 9:106704122 | rs1883025 | 0.79877901 | 0.946917834 |
| 1KG | 15:59828725 | rs11854497 | 0.730884457 | 0.931234743 |
| 1KG | 1:5303445 | rs1326005 | 0.84912884 | 0.994837026 |
| 1KG | 13:83069357 | rs7317763 | 0.99563417 | 1.154653862 |
| 1KG | 20:42475778 | rs1800961 | 0.665286337 | 1.032003698 |
| 1KG | 3:37504640 | rs11716363 | 0.982975875 | 1.200042891 |
| 1KG | 9:4536594 | rs10815017 | 0.851786242 | 1.021300001 |
| 1KG | 16:66459571 | rs2271293 | 0.973749134 | 1.219457916 |
| 1KG | 2:189854831 | rs10207860 | 0.774683658 | 1.045588081 |
| 1KG | 4:165142286 | rs11736266 | 0.880669191 | 1.026947473 |
| 1KG | 18:68641554 | rs11876415 | 0.966961479 | 1.169896614 |
| 1KG | 11:61327359 | rs174547 | 0.972728922 | 1.133409293 |
| 1KG | 12:108379551 | rs2338104 | 0.97244804 | 1.125115127 |
| 1KG | 20:44009909 | rs7679 | 0.964346358 | 1.165361388 |
| 1KG | 5:86468373 | rs2032794 | 0.867339108 | 1.037911737 |
| 1KG | 15:30811243 | rs3743105 | 0.89157199 | 1.035187849 |
| 1KG | 1:228362314 | rs4846914 | 0.8919324 | 1.035200436 |
| 1KG | 19:8375738 | rs2967605 | 0.951864049 | 1.160240069 |
| 1KG | 11:32859894 | rs4755455 | 0.864012372 | 1.050270562 |
| 1KG | 10:24883792 | rs16924889 | 0.823218219 | 1.074262133 |
| 1KG | 13:105365389 | rs4771513 | 0.937272922 | 1.183016146 |
| 1KG | 4:162971300 | rs17639345 | 0.940598755 | 1.151011783 |
| 1KG | 11:116154127 | rs964184 | 0.934028284 | 1.153323116 |
| 1KG | 12:80847264 | rs7973032 | 0.900329289 | 1.208047348 |
| 1KG | 1:57677552 | rs11207037 | 0.897069196 | 1.064270643 |
| 1KG | 9:15279578 | rs471364 | 0.91887667 | 1.152545107 |
| 1KG | 8:19888502 | rs12678919 | 0.906908493 | 1.160503864 |
| 1KG | 18:45421212 | rs4939883 | 0.922413611 | 1.115999534 |
| 1KG | 12:92165329 | rs7973431 | 0.930447499 | 1.093533693 |
| 1KG | 20:4486243 | rs958646 | 0.865104269 | 1.12538676 |
| 1KG | 18:36481096 | rs2469875 | 0.934232358 | 1.080052775 |
| 1KG | 12:123850701 | rs5888 | 0.925099479 | 1.076645294 |
| 1KG | 1:185513773 | rs2453727 | 0.931445644 | 1.077253295 |
| 1KG | 6:57396889 | rs9475939 | 5.76296E−10 | 1030683399 |
| 1KG | 2:71597893 | rs13402171 | 0.882461533 | 1.134100607 |

TABLE 8

| REF | hg_18_BP | Gene (nearby) | SNP | A1 | A2 | FRQ_A(2594) | FRQ_U(4134) |
|---|---|---|---|---|---|---|---|
| 1KG | 8:123521501 | ZHX2 | rs4282579 | A | G | 0.6556 | 0.6888 |
| 1KG | 15:87314645 | MFGE8\|ABHD2 | rs11856826 | T | C | 0.6577 | 0.6267 |
| 1KG | 3:119674993 | IGSF11 | rs12637095 | A | T | 0.8045 | 0.7797 |
| 1KG | 16:85846518 | LOC730018 | rs3748391 | T | G | 0.518 | 0.5459 |
| 1KG | 8:128550166 | POU5F1B | rs10089310 | A | T | 0.1149 | 0.0961 |
| 1KG | 10:88396729 | OPN4\|RPL7AP8 | rs2803544 | A | T | 0.1528 | 0.1335 |
| 1KG | 4:182181154 | LINC00290 | rs9997006 | T | G | 0.3841 | 0.3654 |
| 1KG | 15:59828725 | VPS13C | rs11854497 | A | G | 0.1036 | 0.1139 |
| 1KG | 1:205515927 | CD55\|C4BPAP2 | rs12040406 | T | C | 0.9174 | 0.906 |
| 1KG | 18:34125717 | CELF4 | rs17628762 | A | C | 0.3824 | 0.3426 |
| 1KG | 16:74929641 | CNTNAP4 | rs6564324 | A | G | 0.7163 | 0.7325 |
| 1KG | 11:1762440 | CTSD | rs55911157 | T | C | 0.1489 | 0.1731 |
| 1KG | 9:114121319 | ROD1 | rs10739343 | A | G | 0.2015 | 0.2256 |

| REF | hg_18_BP | Gene (nearby) | OR | SE | P | EA | Z |
|---|---|---|---|---|---|---|---|
| 1KG | 8:123521501 | ZHX2 | 0.8571 | 0.039 | 0.00007708 | G | 3.953302156 |
| 1KG | 15:87314645 | MFGE8\|ABHD2 | 1.1722 | 0.0406 | 0.00009204 | C | −3.910671187 |
| 1KG | 3:119674993 | IGSF11 | 1.1955 | 0.0462 | 0.0001092 | T | −3.869184471 |
| 1KG | 16:85846518 | LOC730018 | 0.8754 | 0.037 | 0.0003254 | T | −3.594191857 |
| 1KG | 8:128550166 | POU5F1B | 1.2349 | 0.0599 | 0.0004286 | A | 3.521816937 |
| 1KG | 10:88396729 | OPN4\|RPL7AP8 | 1.2022 | 0.0536 | 0.000592 | A | 3.435253392 |
| 1KG | 4:182181154 | LINC00290 | 1.2106 | 0.0571 | 0.0008115 | T | 3.3488422 |
| 1KG | 15:59828725 | VPS13C | 0.825 | 0.0618 | 0.001868 | A | −3.110453982 |
| 1KG | 1:205515927 | CD55\|C4BPAP2 | 2.1921 | 0.1528 | 2.786E−07 | C | −5.137376796 |
| 1KG | 18:34125717 | CELF4 | 1.188 | 0.0388 | 0.000008979 | A | 4.440404368 |
| 1KG | 16:74929641 | CNTNAP4 | 0.8354 | 0.0481 | 0.0001849 | G | 3.738801021 |
| 1KG | 11:1762440 | CTSD | 0.7781 | 0.0567 | 0.000009621 | T | −4.425520544 |
| 1KG | 9:114121319 | ROD1 | 0.8504 | 0.0454 | 0.0003545 | A | −3.571826678 |

| REF | hg_18_BP | Gene (nearby) | EA_OR | ci− | ci+ |
|---|---|---|---|---|---|
| 1KG | 8:123521501 | ZHX2 | 1.166725003 | 0.794024723 | 0.925185815 |
| 1KG | 15:87314645 | MFGE8\|ABHD2 | 0.853096741 | 1.082535879 | 1.269290807 |
| 1KG | 3:119674993 | IGSF11 | 0.836470096 | 1.092001781 | 1.308807619 |
| 1KG | 16:85846518 | LOC730018 | 0.8754 | 0.814163271 | 0.941242607 |
| 1KG | 8:128550166 | POU5F1B | 1.2349 | 1.098105031 | 1.38873602 |
| 1KG | 10:88396729 | OPN4\|RPL7AP8 | 1.2022 | 1.08230953 | 1.335371074 |
| 1KG | 4:182181154 | LINC00290 | 1.2106 | 1.082420898 | 1.353957932 |
| 1KG | 15:59828725 | VPS13C | 0.825 | 0.730884457 | 0.931234743 |
| 1KG | 1:205515927 | CD55\|C4BPAP2 | 0.456183568 | 1.624779296 | 2.957510859 |
| 1KG | 18:34125717 | CELF4 | 1.188 | 1.101004804 | 1.281869066 |
| 1KG | 16:74929641 | CNTNAP4 | 1.197031362 | 0.760240364 | 0.917990142 |
| 1KG | 11:1762440 | CTSD | 0.7781 | 0.696259928 | 0.869559751 |
| 1KG | 9:114121319 | ROD1 | 0.8504 | 0.777997124 | 0.929540917 |

TABLE 9

| GENE | SNP | CHR:BP | GENE |
|---|---|---|---|
| HTRA1 | rs10490924 | 10:124204438 | HTRA1 |
| CFH | rs1061170 | 1:194925860 | CFH |
| CFH | rs1410996 | 11:194963556 | CFH |
| CFB | rs641153 | 6:32022159 | CFB |
| C3 | rs2230199 | 19:6669387 | C3 |
| C2 | rs9332739 | 6:32011783 | C2 |
| TIMP3 | rs9621532 | 22:31414511 | TIMP3 |
| LIPC | rs10468017 | 15:56465804 | LIPC |
| CFI | rs10033900 | 4:110878516 | CFI |
| COL10A1, DSE, FRK, TSPY | rs12204816 | 6:116568331 | COL10A1, DSE, FRK, TSPY |
| L1, TSPYL4 | | | L1, TSPYL4 |
| VEGFA | rs4711751 | 6:43936560 | VEGFA |
| CETP | rs3764261 | 16:55550825 | CETP |
| FRK | rs1999930 | 6:116493827 | FRK |
| ABCA1 | rs1883025 | 9:106704122 | ABCA1 |
| FRK/COL10A1 | rs12196141 | 6:116596243 | FRK/COL10A1 |
| HCG27(0) | rs9366769 | 6:31277268 | HCG27(0) |
| LIPC | rs493258 | 15:56475172 | LIPC |
| COL8A1 | rs13095226 | 3:100878962 | COL8A1 |
| TSHZ3(+107.3 kb) | rs2052572 | 19:36639376 | TSHZ3(+107.3 kb) |
| NT5DC1(0) | rs509859 | 6:116529937 | NT5DC1(0) |
| FILIP1L(0)\|C3orf26(0) | rs7626245 | 3:101053451 | FILIP1L(0)\|C3orf26(0) |
| CNTNAP4 | rs8053796 | 16:74921678 | CNTNAP4 |
| MYOM2(−1494 kb) | rs722782 | 8:506479 | MYOM2(−1494 kb) |
| FAM135B(0) | rs10103808 | 8:139212254 | FAM135B(0) |
| OTOL1(+488.4 kb) | rs4256145 | 3:163192835 | OTOL1(+488.4 kb) |
| IRF4(−675.1 kb) | rs9328048 | 6:1324870 | IRF4(−675.1 kb) |
| INTU | rs1443179 | 4:128495772 | INTU |
| MEIS2(+20.97 kb) | chr15:35201758 | 15:35201758 | MEIS2(+20.97 kb) |
| CTSD | rs55911157 | 11:1762440 | CTSD |
| CDH12(−477.3 kb) | rs2883171 | 5:21309600 | CDH12(−477.3 kb) |
| CNTNAP4 | rs6564324 | 16:74929641 | CNTNAP4 |
| ZFAT(+315.9 kb)\|KHDRBS | rs13253938 | 8:136110326 | ZFAT(+315.9 kb)\|KHDRBS |
| 3(−428.6 kb) | | | 3(−428.6 kb) |
| CDH9(−704.3 kb) | chr5:26212150 | 5:26212150 | CDH9(−704.3 kb) |
| TNFRSF10A(+0.332 kb)\|C | rs13278062 | 8:23138916 | TNFRSF10A(+0.332 kb)\|C |
| HMP7(−18.18 kb) | | | HMP7(−18.18 kb) |
| PCDH15(+149.1 kb) | rs61856267 | 10:56380194 | PCDH15(+149.1 kb) |
| GDF6 | rs6982567 | 8:96819457 | GDF6 |
| MRPL19(+489.6 kb) | rs1851808 | 2:76232489 | MRPL19(+489.6 kb) |
| C1orf116, C4BPA, C4BPB, | rs12040406 | 1:205515927 | C1orf116, C4BPA, C4BPB, |
| CD55, CR1, CR2, DAF, PFK | | | CD55, CR1, CR2, DAF, PFK |
| FB2, YOD1 | | | FB2, YOD1 |
| TKT | rs12632671 | 3:53233464 | TKT |
| MOXD1(−134.9 | rs728371 | 6:132523980 | MOXD1(−134.9 |
| kb)\|CTGF(+209.8 kb) | | | kb)\|CTGF(+209.8 kb) |
| GANC(0) | rs12908430 | 15:40375218 | GANC(0) |
| MFGE8(+54.89 kb)\|HAPLN | rs11854658 | 15:87312556 | MFGE8(+54.89 kb)\|HAPLN |
| 3(+72.78 kb)\|ACAN(+92.97 | | | 3(+72.78 kb)\|ACAN(+92.97 |
| kb)\|ABHD2(−119.9 kb) | | | kb)\|ABHD2(−119.9 kb) |
| TMCO1(0) | chr1:163986967 | 1:163986967 | TMCO1(0) |
| SYNGAP1(0) | rs9461856 | 6:33503177 | SYNGAP1(0) |
| USP31(+23 kb)\|UBFD1(−385.3 | rs4967980 | 16:23091095 | USP31(+23 kb)\|UBFD1(−385.3 |
| kb)\|SCNN1B(−130 | | | kb)\|SCNN1B(−130 |
| kb)\|SCNN1G(−10.45 | | | kb)\|SCNN1G(−10.45 |
| kb)\|COG7(−216.2 kb) | | | kb)\|COG7(−216.2 kb) |
| RREB1 | rs11755724 | 6:7063989 | RREB1 |
| WDR35(+148.1 kb)\|TTC32(+236.3 | rs6531212 | 2:20201501 | WDR35(+148.1 kb)\|TTC32(+236.3 |
| kb)\|SDC1(−62.54 | | | kb)\|SDC1(−62.54 |
| kb)\|PUM2(−110.4 | | | kb)\|PUM2(−110.4 |
| kb)\|MATN3(+125.6 | | | kb)\|MATN3(+125.6 |
| kb)\|LAPTM4A(+86.58 kb) | | | kb)\|LAPTM4A(+86.58 kb) |
| PSMD7(−598.4 kb) | rs2127740 | 16:72289810 | PSMD7(−598.4 kb) |
| TMCO5 | rs16965939 | 15:35992085 | TMCO5 |
| MAPK10 | rs28621471 | 4:86877248 | MAPK10 |
| TBX3(+404 kb) | rs11067403 | 12:114010367 | TBX3(+404 kb) |
| B3GALTL(0) | rs1912795 | 13:30736688 | B3GALTL(0) |
| GCNT4(+22.83 kb)\|HMGC | rs12520598 | 5:74385314 | GCNT4(+22.83 kb)\|HMGC |
| R(−283.5 kb) | | | R(−283.5 kb) |
| IGSF11(−421 kb) | rs1915098 | 3:119681130 | IGSF11(−421 kb) |
| ADAMTS16 | rs7720497 | 5:5291813 | ADAMTS16 |
| LSAMP(+563.4 kb) | rs1540819 | 3:118210505 | LSAMP(+563.4 kb) |
| CNTNAP5(0) | rs9308649 | 2:124751486 | CNTNAP5(0) |
| NEDD4L(0) | rs529478 | 18:53934507 | NEDD4L(0) |
| CREG1 | rs1737478 | 1:165795961 | CREG1 |
| | rs12637095 | 3:119674993 | |
| BRUNOL4(+711.5 kb) | rs8091635 | 18:34111545 | BRUNOL4(+711.5 kb) |
| FOXP1(0) | rs2135548 | 3:71584585 | FOXP1(0) |
| SDC3(−13.09 kb)\|PUM1(−75.13 | rs2491146 | 1:31101812 | SDC3(−13.09 kb)\|PUM1(−75.13 |
| kb)\|MATN1(+132.3 | | | kb)\|MATN1(+132.3 |
| kb)\|LAPTM5(+98.56 kb) | | | kb)\|LAPTM5(+98.56 kb) |
| GRM3(−34.61 kb) | rs1358395 | 7:86076557 | GRM3(−34.61 kb) |
| ACVR1B, ACVRL1, bpl_41- | rs7958529 | 12:50881293 | ACVR1B, ACVRL1, bpl_41- |
| 16, C12orf44, GRASP, KRT | | | 16, C12orf44, GRASP, KRT |
| 6A, KRT6B, KRT6C, KRT7, | | | 6A, KRT6B, KRT6C, KRT7, |
| KRT75, KRT80, KRT81, KR | | | KRT75, KRT80, KRT81, KR |
| T82, KRT83, KRT84, KRT85, | | | T82, KRT83, KRT84, KRT85, |
| KRT86, NR4A1 | | | KRT86, NR4A1 |
| EPHA7(−434.6 kb) | rs2037156 | 6:93573222 | EPHA7(−434.6 kb) |
| HMGCLL1(0) | rs4260755 | 6:55456674 | HMGCLL1(0) |
| OSBPL11(0) | chr3:126793536 | 3:126793536 | OSBPL11(0) |
| SPHAR(−384.2 | rs6685751 | 1:227122574 | SPHAR(−384.2 |
| kb)\|RHOU(+173.5 kb) | | | kb)\|RHOU(+173.5 kb) |
| \|RAB4A(−350.9 kb) | | | \|RAB4A(−350.9 kb) |
| PARK2(0) | chr6:162467903 | 6:162467903 | PARK2(0) |
| TAF3(+129.4 kb)\|KIN(+356.1 | rs2798832 | 10:8226099 | TAF3(+129.4 kb)\|KIN(+356.1 |
| kb)\|ITIH2(+394.6 kb)\|ITIH | | | kb)\|ITIH2(+394.6 kb)\|ITIH |
| 5(+477.2 kb)\|GATA3(+68.93 | | | 5(+477.2 kb)\|GATA3(+68.93 |
| kb)\|FLJ45983(+90.65 kb)\| | | | kb)\|FLJ45983(+90.65 kb)\| |
| ATP5C1(+336.3 kb) | | | ATP5C1(+336.3 kb) |
| PPM1B(−116.7 kb)\|LRPPRC(+56.13 | chr2:44132778 | 2:44132778 | PPM1B(−116.7 kb)\|LRPPRC(+56.13 |
| kb) | | | kb) |
| C4BPAL1, C4BPA, CD55 | rs1367068 | 1:205461564 | C4BPAL1, C4BPA, CD55 |
| TMEM133(−218.2 kb)\|PGR(255.8 | rs625761 | 11:100149779 | TMEM133(−218.2 kb)\|PGR(255.8 |
| kb)∥CNTN5(+417.1 kb) | | | kb)∥CNTN5(+417.1 kb) |
| IGF2R(0) | rs1867348 | 6:160376153 | IGF2R(0) |
| P4HA3(0) | rs513683 | 11:73662041 | P4HA3(0) |
| DGKB | rs7783337 | 7:14123511 | DGKB |
| VSTM2A(+132.5 kb)\|SEC6 | rs10225927 | 7:54736904 | VSTM2A(+132.5 kb)\|SEC6 |
| 1G(−50.53 kb)\|EGFR(−317.3 kb) | | | 1G(−50.53 kb)\|EGFR(−317.3 kb) |
| RAP2B(+65.39 kb) | rs16822447 | 3:154434339 | RAP2B(+65.39 kb) |
| TCOF1(0) | chr5:149749881 | 5:149749881 | TCOF1(0) |
| SGCZ(−276.3 | rs1384044 | 8:13715406 | SGCZ(−276.3 |
| kb)\|DLC1(+298.6 kb) | | | kb)\|DLC1(+298.6 kb) |
| RPP30(−238.9 kb)\|HTR7(−107.8 | rs7094579 | 10:92382787 | RPP30(−238.9 kb)\|HTR7(−107.8 |
| kb)\|ANKRD1(−279 kb) | | | kb)\|ANKRD1(−279 kb) |
| CELF4 | rs17628762 | 18:34125717 | CELF4 |
| TCL1B | rs8011890 | 14:95216646 | TCL1B |
| | rs3748391 | 16:85846518 | |
| RELL1(−428.5 kb)\|C4orf19(−291.6 kb) | rs2376241 | 4:36840346 | RELL1(−428.5 kb)\|C4orf19(−291.6 kb) |
| | rs4282579 | 8:123521501 | |
| CTSD(−231.9 kb) | chr11:1768072 | 11:1768072 | CTSD(−231.9 kb) |
| WAPAL(+72.97 kb)\|OPN4(−59.8 kb) | chr10:88344492 | 10:88344492 | WAPAL(+72.97 kb)\|OPN4(−59.8 kb) |
| CACNA1C(0) | rs10848645 | 12:2290505 | CACNA1C(0) |
| MME(−18.73 kb) | rs13094238 | 3:156261403 | MME(−18.73 kb) |
| KIAA1604(+216.2 kb) | chr2:180796214 | 2:180796214 | KIAA1604(+216.2 kb) |
| ANKRD54, CARD10, CAR | rs1079982 | 22:36259705 | ANKRD54, CARD10, CAR |
| MA3, CDC42EP1, FLJ0001 | | | MA3, CDC42EP1, FLJ0001 |
| 7, GALR3, GCAT, GGA1, H1 | | | 7, GALR3, GCAT, GGA1, H1 |
| F0, LGALS1, LGALS2, LRR | | | F0, LGALS1, LGALS2, LRR |
| C62, MFNG, NOL12, PDXP, | | | C62, MFNG, NOL12, PDXP, |
| PSCD4, RAC2, SH3BP1, T | | | PSCD4, RAC2, SH3BP1, T |
| RIOBP | | | RIOBP |
| FLJ37543(+74.42 kb) | rs9291737 | 5:61112540 | FLJ37543(+74.42 kb) |
| EFNA5(0) | rs164700 | 5:106936632 | EFNA5(0) |
| FUT3 | rs3760775 | 19:5792356 | FUT3 |
| | rs11856826 | 15:87314645 | |
| SLC18A1 | rs2270637 | 8:20081107 | SLC18A1 |
| PPARGC1A(−214.7 kb) | chr4:23188024 | 4:23188024 | PPARGC1A(−214.7 kb) |
| RAG2(+1021 kb) | rs10768315 | 11:37597461 | RAG2(+1021 kb) |
| CDGAP(0) | rs9713311 | 3:120558458 | CDGAP(0) |
| MBP | rs1789110 | 18:72988032 | MBP |
| SPATA20 | rs8076470 | 17:45980827 | SPATA20 |
| ARHGAP12(−98.91 kb) | rs2277252 | 10:32036318 | ARHGAP12(−98.91 kb) |
| KCNN2(−80.47 kb) | chr5:113645446 | 5:113645446 | KCNN2(−80.47 kb) |
| NDUFS4(−271 | rs251525 | 5:52621268 | NDUFS4(−271 |
| kb)\|MOCS2(+179.9 kb)\| | | | kb)\|MOCS2(+179.9 kb)\| |
| ITGA2(+194.9 kb)\|ITGA1(+336 | | | ITGA2(+194.9 kb)\|ITGA1(+ 336 |
| kb)\|FST(−191.1 kb) | | | kb)\|FST(−191.1 kb) |
| ITGA9(0) | rs11716363 | 3:37504640 | ITGA9(0) |
| AGL(+4.508 kb)\|SLC35A3(−41.45 kb) | chr1:100166675 | 1:100166675 | AGL(+4.508 kb)\|SLC35A3(−41.45 kb) |
| NDFIP2(0) | rs17071512 | 13:78960018 | NDFIP2(0) |
| PDGFD(0) | rs12798294 | 11:103362906 | PDGFD(0) |
| ZWINT(−308.1 kb) | chr10:57479107 | 10:57479107 | ZWINT(−308.1 kb) |
| FOXF1(−172.4 kb) | rs12926103 | 16:84929276 | FOXF1(−172.4 kb) |
| RREB1(0) | rs1360751 | 6:7152544 | RREB1(0) |
| ROD1(0) | rs10739343 | 9:114121319 | ROD1(0) |
| TRA2A, IGF2BP3, CLK2P | rs10280782 | 7:23550807 | TRA2A, IGF2BP3, CLK2P |
| | rs11854497 | 15:59828725 | |
| TUBB1(−48.54 kb)\|TH1L(−10.54 | | 20:56979160 | TUBB1(−48.54 kb)\|TH1L(−10.54 |
| kb)\|STX16(+291.2 kb) | rs235195 | | kb)\|STX16(+291.2 kb) |
| \|SLMO2(−62.43 kb)\|NPEPL1(+254.9 | | | \|SLMO2(−62.43 kb)\|NPEPL1(+254.9 |
| kb)\|GNAS(+59.52 kb)\|CTSZ | | | kb)\|GNAS(+59.52 kb)\|CTSZ |
| (−24.47 kb)\|ATP5E(−57.97 kb) | | | (−24.47 kb)\|ATP5E(−57.97 kb) |
| AEBP2(+312 kb) | rs1492116 | 12:19878442 | AEBP2(+312 kb) |
| KLF6(+127.8 kb) | rs11592003 | 10:3945290 | KLF6(+127.8 kb) |
| SDCCAG10(0) | rs10067691 | 5:64292165 | SDCCAG10(0) |
| ODZ2(−1511 kb) | chr5:165133520 | 5:165133520 | ODZ2(−1511 kb) |
| NUTF2(0) | rs2271293 | 16:66459571 | NUTF2(0) |
| HNF4A(0) | rs1800961 | 20:42475778 | HNF4A(0) |
| CASP4(−133.1 kb) | chr11:104185708 | 11:104185708 | CASP4(−133.1 kb) |
| ZNF692(+10.26 kb)\|SH3B | rs6587759 | 1:247130153 | ZNF692(+10.26 kb)\|SH3B |
| P5L(+43.38 kb)\|PGBD2(−36.91 kb)\| | | | P5L(+43.38 kb)\|PGBD2(−36.91 kb)\| |
| MRPS24 | rs2730613 | 7:43880667 | MRPS24 |
| SGCZ(0) | rs10503493 | 8:14247492 | SGCZ(0) |
| PCIF1(0) | rs7679 | 20:44009909 | PCIF1(0) |
| XYLT1(0) | chr16:17310751 | 16:17310751 | XYLT1(0) |
| TBX20(+335.4 kb)\|SEPT7(−212 | rs343718 | 7:35595154 | TBX20(+335.4 kb)\|SEPT7(−212 |
| kb)\|HERPUD2(−43.64 kb) | | | kb)\|HERPUD2(−43.64 kb) |
| FSTL5(0) | rs17639345 | 4:162971300 | FSTL5(0) |
| SLC1A1 | rs10815017 | 9:4536594 | SLC1A1 |
| | rs2803544 | 10:88396729 | |
| | rs9997006 | 4:182181154 | |
| | rs10233234 | 7:23498756 | |
| DAB1(−16.87 | rs1418473 | 1:57219300 | DAB1(−16.87 |
| kb)\|C8B(+15.02 kb)\|C | | | kb)\|C8B(+15.02 kb)\|C |
| 8A(+62.82 kb) | | | 8A(+62.82 kb) |
| FAM113B | rs11183802 | 12:45893566 | FAM113B |
| | rs10089310 | 8:128550166 | |
| ZC3H7A(+30.67 kb)\|TXND | rs2024393 | 16:11814579 | ZC3H7A(+30.67 kb)\|TXND |
| C11(+70.43 kb)\|TNFRSF1 | | | C11(+70.43 kb)\|TNFRSF1 |
| 7(−151.9 | | | 7(−151.9 |
| kb)\|SNN(+134.1 kb)\| | | | kb)\|SNN(+134.1 kb)\| |
| RUNDC2A(−163.5 | | | RUNDC2A(−163.5 |
| kb)\|RSL1D1(−20.98 | | | kb)\|RSL1D1(−20.98 |
| kb)\|GSPT1(−54.91 kb) | | | kb)\|GSPT1(−54.91 kb) |
| LILRB3 | rs7260457 | 19:59425996 | LILRB3 |
| PLN(+129.5 kb)\|MCM9(−155.7 | chr6:119117772 | 6:119117772 | PLN(+129.5 kb)\|MCM9(−155.7 |
| kb)\|ASF1A(−145.9 kb) | | | kb)\|ASF1A(−145.9 kb) |
| | rs7973431 | 12:92165329 | |
| ELTD1(+115.3 kb) | rs17408651 | 1:79360392 | ELTD1(+115.3 kb) |
| DDAH1(+53.95 kb)\|CYR61 | chr1:85757359 | 1:85757359 | DDAH1(+53.95 kb)\|CYR61 |
| (−61.69 kb) | | | (−61.69 kb) |
| upstream AK125078 | rs1326005 | 1:5303445 | upstream AK125078 |
| | rs11736266 | 4:165142286 | |
| A2ML1(0) | chr12:8919954 | 12:8919954 | A2ML1(0) |
| FADS1(0) | rs174547 | 11:61327359 | FADS1(0) |
| | rs4939883 | 18:45421212 | |
| ARL5B(+385.1 kb) | rs11596472 | 10:19392088 | ARL5B(+385.1 kb) |
| | rs964184 | 11:116154127 | |
| ATP8B4(0) | chr15:47957127 | 15:47957127 | ATP8B4(0) |
| downstream AK058053 | rs4771513 | 13:105365389 | downstream AK058053 |
| | rs7317763 | 13:83069357 | |
| OR7C1(+3.191 kb)\|OR7A5 | chr19:14775139 | 19:14775139 | OR7C1(+3.191 kb)\|OR7A5 |
| (−23 kb) | | | (−23 kb) |
| upstream QSER1 | rs4755455 | 11:32859894 | upstream QSER1 |
| | rs7973032 | 12:80847264 | |
| MGAT5B(+25.65 kb) | chr17:72483712 | 17:72483712 | MGAT5B(+25.65 kb) |
| COL5A2(alpha 2 type V | rs10207860 | 2:189854831 | COL5A2(alpha 2 type V |
| collagen preprotein) | | | collagen preprotein) |
| SCARB1 | rs5888 | 12:123850701 | SCARB1 |
| BC034940 | rs2032794 | 5:86468373 | BC034940 |
| NETO1 | rs11876415 | 18:68641554 | NETO1 |
| GREM1(0) | rs3743105 | 15:30811243 | GREM1(0) |
| | rs2469875 | 18:36481096 | |
| ODZ3(−1293 kb) | chr4:182189551 | 4:182189551 | ODZ3(−1293 kb) |
| ZBTB20(+12.48 kb) | chr3:116361297 | 3:116361297 | ZBTB20(+12.48 kb) |
| SESN1(0) | rs13192030 | 6:109470664 | SESN1(0) |
| ZHX2(−341.2 kb) | chr8:123521912 | 8:123521912 | ZHX2(−341.2 kb) |
| GALNT2(0) | rs4846914 | 1:228362314 | GALNT2(0) |
| PRR5(−270.7 | rs135912 | 22:43172593 | PRR5(−270.7 |
| kb)\|PARVB(+276.2 | | | kb)\|PARVB(+276.2 |
| kb)\|PARVG(+238.7 kb)\|LDO | | | kb)\|PARVG(+238.7 kb)\|LDO |
| C1L(−94.52 | | | C1L(−94.52 |
| kb)\|KIAA1644(+132.5 kb) | | | kb)\|KIAA1644(+132.5 kb) |
| FGGY(−58.3 kb) | chr1:59476907 | 1:59476907 | FGGY(−58.3 kb) |
| USH2A(+36.74 kb)\|TGFB2 | rs6679773 | 1:214700097 | USH2A(+36.74 kb)\|TGFB2 |
| (−1886 kb)\|SPATA17(−1171 | | | (−1886 kb)\|SPATA17(−1171 |
| kb)\|RRP15(−1825 kb)\|PTPN14(+1909 | | | kb)\|RRP15(−1825 kb)\|PTPN14(+1909 |
| kb)\|KCTD3(+838.3 kb)\|KCNK | | | kb)\|KCTD3(+838.3 kb)\|KCNK |
| 2(+1223 kb)\|GPATCH2(−970.4 | | | 2(+1223 kb)\|GPATCH2(−970.4 |
| kb)\|ESRRG(−43.11 | | | kb)\|ESRRG(−43.11 |
| kb)\|CENPF(+1796 kb) | | | kb)\|CENPF(+1796 kb) |
| | rs958646 | 20:4486243 | |
| FAM19A5 | rs5771717 | 22:47467676 | FAM19A5 |
| TNRC6C(+225.5 kb)\|TMC8 | rs16971055 | 17:73838135 | TNRC6C(+225.5 kb)\|TMC8 |
| (+187.5 kb)\|TMC6(+198.1 | | | (+187.5 kb)\|TMC6(+198.1 |
| kb)\|TK1(+143.3 kb)\|SYNGR | | | kb)\|TK1(+143.3 kb)\|SYNGR |
| 2(+157.5 kb)\|SOCS3(−26.32 | | | 2(+157.5 kb)\|SOCS3(−26.32 |
| kb)\|PGS1(−48.19 | | | kb)\|PGS1(−48.19 |
| kb)\|BIRC5(+104.8 kb) | | | kb)\|BIRC5(+104.8 kb) |
| \|AFMID(+122.8 kb) | | | \|AFMID(+122.8 kb) |
| | rs16924889 | 10:24883792 | |
| WDFY3(+99.94 kb) | chr4:86206508 | 4:86206508 | WDFY3(+99.94 kb) |
| NPLOC4(0) | rs4073997 | 17:77136846 | NPLOC4(0) |
| ISX(+77.68 kb)\|HMG2L1(−92.42 | chr22:33891064 | 22:33891064 | ISX(+77.68 kb)\|HMG2L1(−92.42 |
| kb) | | | kb) |
| KCTD10(0) | rs2338104 | 12:108379551 | KCTD10(0) |
| RHO(−139 kb)\|MBD4(−41.36 | rs12632105 | 3:130591125 | RHO(−139 kb)\|MBD4(−41.36 |
| kb)\|IFT122(−50.53 | | | kb)\|IFT122(−50.53 |
| kb)\|H1FX(+73.32 kb)\| | | | kb)\|H1FX(+73.32 kb)\| |
| COPG(+111.8 kb) | | | COPG(+111.8 kb) |
| DAB1(0) | rs11207037 | 1:57677552 | DAB1(0) |
| | rs12678919 | 8:19888502 | |
| DKFZp667G2110(0) | rs7623235 | 3:99135842 | DKFZp667G2110(0) |
| HIBADH(−56.23 kb) | rs17155281 | 7:27475356 | HIBADH(−56.23 kb) |
| | rs9475939 | 6:57396889 | |
| ROBO3(−27.68 kb)\|C11orf61(+37.3 | chr11:124212812 | 11:124212812 | ROBO3(−27.68 kb)\|C11orf61(+37.3 |
| kb) | | | kb) |
| | rs2967605 | 19:8375738 | |
| INDO(+194.4 kb) | chr8:40099493 | 8:40099493 | INDO(+194.4 kb) |
| TAS2R42(+2.424 kb) | chr12:11233234 | 12:11233234 | TAS2R42(+2.424 kb) |
| ENPP3(0) | rs5015852 | 6:132002096 | ENPP3(0) |
| | rs2453727 | 1:185513773 | |
| TTC39B(0) | rs471364 | 9:15279578 | TTC39B(0) |
| DYSF(0) | rs13402171 | 2:71597893 | DYSF(0) |

What is claimed is:

1. A method of screening for age-related macular degeneration (AMD) in a human subject, the method comprising:
determining a risk of a subject developing AMD by analyzing a sample obtained from the subject for the presence of at least one single nucleotide polymorphism (SNP) in the vascular endothelial growth factor A (VEGFA) gene region, the at least one SNP comprising rs4711751 or a proxy for rs4711751, the presence of a SNP being indicative of an increased risk of the subject developing AMD.

2. A method of screening for age-related macular degeneration (AMD) in a human subject, the method comprising:
determining a risk of AMD progression in a subject by analyzing a sample obtained from the subject for the presence of at least one single nucleotide polymorphism (SNP) in the vascular endothelial growth factor A (VEGFA) gene region, the at least one SNP comprising rs4711751 or a proxy for rs4711751, the presence of a SNP being indicative of an increased risk of the subject developing an advanced form of AMD.

3. A method of screening for age-related macular degeneration (AMD) in a human subject, the method comprising:
determining a risk of a subject developing AMD by analyzing a sample obtained from the subject for the presence of at least one single nucleotide polymorphism (SNP) in the growth/differentiation factor 6 (GDF6) gene region, the at least one SNP comprising rs6982567 or a proxy for rs6982567, the presence of a SNP being indicative of an increased risk of the subject developing AMD.

4. A method of screening for age-related macular degeneration (AMD) in a human subject, the method comprising:
determining a risk of AMD progression in a subject by analyzing a sample obtained from the subject for the presence of at least one single nucleotide polymorphism (SNP) in the growth/differentiation factor 6 (GDF6) gene region, the at least one SNP comprising rs6982567 or a proxy for rs6982567, the presence of a SNP being indicative of an increased risk of the subject developing an advanced form of AMD.

5. A method of screening for age-related macular degeneration (AMD) in a human subject, the method comprising:
determining a risk of a subject developing AMD by analyzing a sample obtained from the subject for the presence of at least one single nucleotide polymorphism (SNP) selected from the group consisting of rs4711751, rs1999930, rs13278062, rs1912795, rs2270637, rs6982567, rs12040406, rs1367068, rs1079982, rs59795197, rs1443179, rs7720497, rs61800454, or a proxy therefor, the presence of a SNP being indicative of an increased risk of the subject developing AMD or developing an advanced form of AMD.

6. A method of screening for age-related macular degeneration (AMD) in a human subject, the method comprising:
determining a risk of AMD progression in a subject by analyzing a sample obtained from the subject for the presence of at least one single nucleotide polymorphism (SNP) identified in Tables 3-9, or a proxy therefor, the presence of a SNP being indicative of an increased risk of the subject developing AMD or developing an advanced form of AMD.

7. The method of claim 1 wherein the analyzing comprises (i) combining a nucleic acid sample from the subject with one or more polynucleotide probes capable of hybridizing selectively to a VEGFA gene allele, or a proxy therefor, and (ii) detecting the presence or absence of hybridization.

8. The method of claim 6 wherein the analyzing comprises (i) combining a nucleic acid sample from the subject with one or more polynucleotide probes capable of hybridizing selectively to a GDF6 gene allele, or a proxy therefor, and (ii) detecting the presence or absence of hybridization.

9. The method of claim 7 wherein the probes are oligonucleotides capable of priming polynucleotide synthesis in an amplification reaction.

10. The method of claim 1, wherein the subject is asymptomatic at the time of screening.

11. The method of claim 1, wherein the SNP is detected in a haplotype comprising the SNP.

12. The method of claim 1, comprising screening for a specific subtype of AMD.

13. The method of claim 12, wherein the subtype is selected from the group consisting of early AMD, geographic atrophy, exudative AMD (CNV or neovascular disease), and combinations thereof.

14. The method of claim 1, wherein the presence of at least one SNP is determined using a microarray.

15. The method of claim 1, wherein the presence of at least one SNP is determined by sequencing.

16. The method of claim 1, wherein the proxy is in linkage disequilibrium with the SNP.

17. A diagnostic system comprising:
an array of polynucleotides comprising one or more of SEQ ID NOS:1-16, the polynucleotides comprising at least six or more contiguous nucleotides, and the polynucleotides comprising an allelic polymorphism,
an array reader, an image processor, a database having AMD allelic data records and patient information records, a processor, and an information output,
wherein the system compiles and processes patient data and outputs information relating to the statistical probability of the patient developing AMD.

18. A method of using the diagnostic system of claim 17, comprising contacting a subject sample or portion thereof to the diagnostic array under high stringency hybridization conditions; inputting patient information into the system; and obtaining from the system information relating to the statistical probability of the patient developing AMD.

19. A method for diagnosing risk of AMD or severe forms of AMD in a human subject, the method comprising combining genetic risk with behavioral risk, wherein the genetic risk is determined by detecting in a sample obtained from a subject the presence or absence of a single nucleotide polymorphism SNP listed in Tables 3, 4, 5, 6, 7, 8, 9, or 10, or proxy therefor, wherein the presence of the allele is indicative of an increased risk of the subject developing AMD or a severe form of AMD.

20. The method of claim 19, wherein a behavioral risk is assessed by determining if the subject exhibits a behavior or trait selected from the group consisting of: obesity, smoking, vitamin and dietary supplement intake, use of alcohol or drugs, poor diet and a sedentary lifestyle.
